(19) United States
(12) Patent Application Publication
(10) Pub. No.: US 2025/0312439 A1
(43) Pub. Date: Oct. 9, 2025

(54) VACCINE FOR PREVENTING OR TREATING CORONAVIRUS INFECTION

(71) Applicant: PHION THERAPEUTICS LTD, Belfast, Antrim (GB)

(72) Inventors: Helen MCCARTHY, Belfast, Antrim (IE); Cian Michael MCCRUDDEN, Belfast (GB)

(21) Appl. No.: 18/866,792

(22) PCT Filed: May 17, 2023
(86) PCT No.: PCT/EP2023/063244

§ 371 Date: Nov. 18, 2024

(30) Foreign Application Priority Data
May 18, 2022 (GB) 2207281.3

(51) Int. Cl. A61K 39/215; A61K 39/00; A61K 39/385; A61P 37/04

(52) U.S. Cl. CPC A61K 39/215; A61K 39/385; A61P 37/04; A61K 2039/53

(57) ABSTRACT

The present specification relates to vaccines comprising an mRNA polynucleotide encoding a coronavirus antigen; and an amphipathic cell penetrating RALA peptide.

### FIELD

The present specification relates to vaccines. In particular, the specification relates to vaccines comprising an mRNA polynucleotide encoding a coronavirus antigen and an amphipathic cell penetrating peptide from the RALA family of peptides. The specification further relates to methods of preparing such vaccines and to their use in therapy.

### BACKGROUND

Vaccines involve introducing antigens—a substance that the immune system will attack—either directly or indirectly into the body, typically via injection. There are several different types of vaccines, including inactivated vaccines; live-attenuated vaccines; messenger ribonucleic acid (mRNA) vaccines; subunit, recombinant, polysaccharide, and conjugate vaccines; toxoid vaccines; and viral vector vaccines. These different vaccines introduce the antigen into the body in different ways.

mRNA vaccines convey instructions to cells to make antigens, which in turn provoke an immune response. The mRNA selected encodes for an antigen of the infection and/or disease to be treated. mRNA vaccines have several benefits compared to other types of vaccines including shorter manufacturing times, and since mRNA cannot be incorporated into the cellular genome, they carry no risk of causing the disease in the person getting vaccinated. However, unmodified synthetic mRNA is not stable, and the mRNA itself can stimulate the activation of an unwanted immune response. Chemical modifications on the ribose, the RNA termini, and nucleobases may therefore be required to improve stability and reduce immunogenicity (see for example Gao et al., Acta. Biomater. 2021 Sep. 1; 131: 1-15).

The antigen delivery system employed in mRNA vaccination, plays a key role in determining the immune response. The ratio of carrier:mRNA also plays a role—mRNA molecules are negatively charged polynucleotides and the carrier needs both to protect the cargo from degradation as well as deliver it to the correct target cells. Optimisation of the carrier:mRNA ratio facilitates production of nanoparticles with suitable size and charge characteristics to ensure uptake by antigen-presenting cells.

Coronaviruses are a group of RNA viruses that cause disease, particularly respiratory tract infections, in mammals and birds. In humans coronaviruses are responsible for severe acute respiratory syndrome (SARS), Middle East respiratory syndrome (MERS) and Coronavirus disease 2019 (COVID-19). There are four main sub-groupings of coronaviruses: alpha, beta, gamma, and delta.

COVID-19 is caused by severe acute respiratory syndrome coronavirus 2 (SARS-CoV-2), a relatively new coronavirus causing high morbidity and mortality. SARS-CoV-2 spread rapidly around the world infecting nearly 500 million people and causing more than 6 million mortalities by April 2022. New cases of COVID-19 infection are on the rise and are still increasing rapidly.

As of March 2022, 10 SARS-CoV-2 vaccines have been approved worldwide. These fall into the broad categories of mRNA-based vaccines; non-replicating viral vectors; inactivated vaccines; and protein subunit vaccines (WHO COVID-19 vaccine tracker). Thus far, the search for vaccine antigens to SARS-CoV-2 has relied heavily on the Spike (S) protein, as it is the S protein that interacts with the host ACE2 receptor. mRNA-1273 (manufactured by Moderna, Inc.), for example, is a novel lipid nanoparticle (LNP)-encapsulated mRNA-based vaccine that encodes for a full-length, prefusion stabilized S protein of SARS-CoV-2. These vaccine strategies will be instrumental in the future to prevent future infections, however there is the risk that through a high mutation rate, coronaviral infections will exist for many years to come.

To date, little attention has been paid to the antigenic properties of the other structural proteins of SARS-CoV2, which could limit the effectiveness of emerging vaccines. As well as the S protein, coronavirus contains three other structural proteins, Membrane (M), Envelope (E), and Nucleoprotein (N) within the viral envelope that could provide the basis for other vaccination approaches. Lee et al (F1000Res, 2020 Feb. 25; 9:145) for example reported a list of immunogenic peptides that could be used as potential targets for vaccine development. These immunogenic peptides have origins not only in the S protein, but also in the M and N proteins.

The RALA family of peptides are amphipathic peptides composed of repeating RALA units that are capable of overcoming biological barriers to gene delivery, both in vitro and in vivo. The term “RALA” has been used inconsistently in the literature, but typically refers to an amphipathic peptide or group of peptides composed of repeating RALA units generally of less than approximately 50 amino acid residues. Cohen-Avrahami et al. (J. Phys. Chem. B 2011, 115:10 189-1097 and Colloids and Surfaces B: Biointerfaces 77 (2010) 131-138) disclose an amphipathic 16-mer peptide referred to as “RALA”. Faranack et al., (Biomacromolecules 2013, 14, 2033-2040) use the term “RALA” to describe a 30-mer RALA peptide, as does McCarthy et al. (Journal of Controlled Release 189 (2014) 141-149) but for a different 30-mer peptide. WO 2014/087023 and WO 2015/189205 defined the term “RALA” as a generic term for a group of peptides falling within the scope of the invention as described therein.

The RALA family of peptides have been used to deliver genetic material such as plasmid DNA (McCarthy H O et al., J Control Release, 2014 Sep. 10; 189:141-9; and Ali A A et al., Nanomedicine, 2017 April; 13(3):921-932), mRNA (Udhayakumar et al., Adv. Healthc. Mater. 2017 July; 6(13)), siRNA (Mulholland E. J. et al., J Control Release, 2019 Dec. 28; 316:53-65), and small molecules such as bisphosphonates (Jena L N et al., J. Nanobiotechnology. 2021 May 4; 19(1):127), and calcium phosphates (Sathy B. N. et al., J. Mater. Chem. B. 2017 Mar. 7; 5(9):1753-1764).

The specific RALA peptide WEARLARALARALARHLARALARALRACEA (SEQ ID_No 1) is a 30 amino acid, non-toxic, peptide with a +6 electric charge at a physiological pH that converts to a +8 helical cell penetrating conformation at acidic conditions found inside the endosome of a cell. When complexed with certain payloads, e.g. DNA and mRNA, in water it has been shown to be capable of spontaneous self-assembly into nanoparticles (McCarthy et al. 2014 and Udhayakumar et al. 2017). This pH dependent change allows for escape of the peptide and the cargo within a cell, resulting in highly efficiency cellular entry and cargo delivery, without any associated toxicity at the physiological pH of 7.4 outside the cell. The nanoparticles have been shown to be extremely stable at a range of temperatures and over time, and RALA peptides do not themselves provoke an immunological response.

Udhayakumar et al. (Udhayakumar et al. 2017) evaluated the capacity of this RALA peptide in mRNA nanocomplexes to prime CD8+ T cells by immunising mice with RALA complexed mRNA encoding the model antigen ovalbumin (the main protein found in egg white). Udhayakumar et al. concluded that multiple different mRNA nucleotide modifications and an N:P ratio of 10 (i.e. ratio of positively charged nitrogen atoms in the peptide to negatively charged phosphates in the mRNA backbone) were needed for optimum complexation of Ova mRNA and to enable efficient CD8⁺ T cell priming.

The present specification describes vaccines comprising an mRNA polynucleotide encoding at least one coronavirus antigen (or fragments thereof) and a specific RALA peptide. These vaccines result in intracellular delivery of the mRNA cargo, protecting it from degradation. Furthermore, the RALA complexed mRNA nanoparticles can be readily lyophilized (retaining functionality), are stable and readily reconstituted, do not require cold chain storage, and are relatively inexpensive to manufacture. Where the vaccines encode more than one antigen (or fragments thereof) this has the potential to maximise the response to the vaccine.

### SUMMARY

This specification describes, in part, a vaccine, comprising:
i) an mRNA polynucleotide comprising an open reading frame encoding a coronavirus antigen or an immunogenic fragment thereof; and
ii) an amphipathic cell penetrating peptide comprising or consisting of the amino acid sequence WEARLARALARALARHLARALARALRACEA (SEQ ID_No 1) or a sequence with at least 80% sequence identity or homology.

This specification also describes, in part, vaccines as described herein for use in therapy, particularly for use in the treatment of coronavirus infections.

This specification also describes, in part, methods of manufacture of vaccines as described herein.

### DETAILED DESCRIPTION OF THE INVENTION

Many embodiments of the invention are detailed throughout the specification and will be apparent to a reader skilled in the art. The invention is not to be interpreted as being limited to any of the recited embodiments.

“A” (or “an”) means “at least one”. In any embodiment where “a” is used to denote a given material or element, “a” may mean one.

“Comprising” means that a given material or element may contain other materials or elements. In any embodiment where “comprising” is mentioned the given material or element may be formed of at least 10% w/w, at least 20% w/w, at least 30% w/w, or at least 40% w/w of the material or element. In any embodiment where “comprising” is mentioned, “comprising” may also mean “consisting of” (or “consists of”) or “consisting essentially of” (or “consists essentially of”) a given material or element.

“Consisting of” or “consists of” means that a given material or element is formed entirely of the material or element. In any embodiment where “consisting of” or “consists of” is mentioned, the given material or element may be formed of 100% w/w of the material or element.

“Consisting essentially of” or “consists essentially of” means that a given material or element consists almost entirely of that material or element. In any embodiment where “consisting essentially of” or “consists essentially of” is mentioned the given material or element may be formed of at least 50% w/w, at least 60% w/w, at least 70% w/w, at least 80% w/w, at least 90% w/w, at least 95% w/w or at least 99% w/w of the material or element.

In any embodiment where “is” or “may be” is used to define a material or element, “is” or “may be” may mean the material or element “consists of” or “consists essentially of” the material or element.

Claims are embodiments.

Embodiments may be combined.

mRNA Polynucleotide

A polynucleotide is a compound and/or substance that comprises a polymer of nucleotides (nucleotide monomers). The polynucleotides of the present disclosure function as messenger RNA (mRNA). “Messenger RNA” refers to any polynucleotide that encodes a polypeptide (both naturally-occurring (wild-type) and non-naturally-occurring) and can be translated to produce the encoded polypeptide in vitro, in vivo, in situ or ex vivo.

The basic components of an mRNA molecule typically include at least one open reading frame, a 5′ untranslated region (UTR), a 3′ UTR, a 5′ cap and a poly-A tail. The mRNA polynucleotides described herein function as mRNA but may differ from wild-type mRNA in both functional and/or structural design features.

A “5′ untranslated region” (5′UTR) refers to a region of an mRNA that is directly upstream (i.e., 5′) from the start codon (i.e., the first codon of an mRNA transcript translated by a ribosome) that does not encode a polypeptide.

A “3′ untranslated region” (3′UTR) refers to a region of an mRNA that is directly downstream (i.e., 3′) from the stop codon (i.e., the codon of an mRNA transcript that signals a termination of translation) that does not encode a polypeptide.

A “poly-A tail” is a region of mRNA that is downstream, e.g., directly downstream (i.e., 3′), from the 3′ UTR that contains multiple, consecutive adenosine monophosphates. A poly-A tail may contain 10 to 150 adenosine monophosphates. In one embodiment, a poly-A tail contains 50 to 150 adenosine monophosphates. In one embodiment, a poly-A tail contains 75 to 125 adenosine monophosphates. In one embodiment, a poly-A tail contains about 120 adenosine monophosphates. In one embodiment, a poly-A tail contains 120 adenosine monophosphates. In one embodiment, a poly-A tail contains about 80 adenosine monophosphates. In one embodiment, a poly-A tail contains 80 adenosine monophosphates. In a relevant biological setting (e.g., in cells, in vivo) the poly-A tail functions to protect mRNA from enzymatic degradation, e.g., in the cytoplasm, and aids in transcription termination, export of the mRNA from the nucleus, and translation.

In one embodiment the vaccine comprises an mRNA polynucleotide comprising an open reading frame encoding a coronavirus antigen or an immunogenic fragment thereof wherein the mRNA polynucleotide comprises 200 to 5,000 nucleotides.

In one embodiment the vaccine comprises an mRNA polynucleotide comprising an open reading frame encoding a coronavirus antigen or an immunogenic fragment thereof wherein the mRNA polynucleotide comprises 800 to 4,500 nucleotides.

In one embodiment the vaccine comprises an mRNA polynucleotide comprising an open reading frame encoding a coronavirus S protein or an immunogenic fragment thereof wherein the mRNA polynucleotide comprises 3,800 to 4,500 nucleotides.

In one embodiment the vaccine comprises an mRNA polynucleotide comprising an open reading frame encoding a coronavirus S protein or an immunogenic fragment thereof wherein the mRNA polynucleotide comprises 4,000 to 4,200 nucleotides.

In one embodiment the vaccine comprises an mRNA polynucleotide comprising an open reading frame encoding a coronavirus E protein or an immunogenic fragment thereof wherein the mRNA polynucleotide comprises 250 to 750 nucleotides.

In one embodiment the vaccine comprises an mRNA polynucleotide comprising an open reading frame encoding a coronavirus E protein or an immunogenic fragment thereof wherein the mRNA polynucleotide comprises 450 to 550 nucleotides.

In one embodiment the vaccine comprises an mRNA polynucleotide comprising an open reading frame encoding a coronavirus M protein or an immunogenic fragment thereof wherein the mRNA polynucleotide comprises 700 to 1200 nucleotides.

In one embodiment the vaccine comprises an mRNA polynucleotide comprising an open reading frame encoding a coronavirus M protein or an immunogenic fragment thereof wherein the mRNA polynucleotide comprises 900 to 1000 nucleotides.

In one embodiment the vaccine comprises an mRNA polynucleotide comprising an open reading frame, a 5′ UTR, a 3′ UTR, a 5′ cap and a poly-A tail encoding a coronavirus antigen or an immunogenic fragment thereof.

In one embodiment the vaccine comprises an mRNA polynucleotide comprising an open reading frame, a 5′ UTR, a 3′ UTR, a 5′ cap and a poly-A tail encoding a coronavirus antigen or an immunogenic fragment thereof wherein the 5′ UTR, the 3′ UTR, and the poly-A tail comprise about 200-400 nucleotides.

In one embodiment the vaccine comprises an mRNA polynucleotide comprising an open reading frame, a 5′ UTR, a 3′ UTR, a 5′ cap and a poly-A tail encoding a coronavirus antigen or an immunogenic fragment thereof wherein the 5′ UTR, the 3′ UTR, and the poly-A tail comprise about 250-320 nucleotides.

In one embodiment the vaccine comprises an mRNA polynucleotide comprising an open reading frame, a 5′ UTR, a 3′ UTR, a 5′ cap and a poly-A tail encoding a coronavirus antigen or an immunogenic fragment thereof wherein the 5′ UTR, the 3′ UTR, and the poly-A tail comprise about 280 nucleotides.

### An Open Reading Frame

An “open reading frame” (ORF) is a continuous stretch of polynucleotide beginning with a start codon (e.g., methionine (AUG)), and ending with a stop codon (e.g., UAA, UAG or UGA). It encodes, and has the potential to be translated into, a polypeptide (in this case the antigen or the immunogenic fragment thereof). It is also known as a coding region.

In one embodiment the vaccine comprises an mRNA polynucleotide comprising an open reading frame encoding a coronavirus antigen or an immunogenic fragment thereof wherein the open reading frame comprises 500-4000 nucleotides.

In one embodiment the vaccine comprises an mRNA polynucleotide comprising an open reading frame encoding a coronavirus antigen or an immunogenic fragment thereof wherein the open reading frame comprises 600-3900 nucleotides.

In one embodiment the vaccine comprises an mRNA polynucleotide comprising an open reading frame encoding a coronavirus S protein or an immunogenic fragment thereof wherein the open reading frame comprises 3500-4100 nucleotides.

In one embodiment the vaccine comprises an mRNA polynucleotide comprising an open reading frame encoding a coronavirus S protein or an immunogenic fragment thereof wherein the open reading frame comprises 3800-3900 nucleotides.

In one embodiment the vaccine comprises an mRNA polynucleotide comprising an open reading frame encoding a coronavirus S protein or an immunogenic fragment thereof wherein the open reading frame consists of 3822 nucleotides.

In one embodiment the vaccine comprises an mRNA polynucleotide comprising an open reading frame encoding a coronavirus S protein or an immunogenic fragment thereof wherein the open reading frame consists of 3813 nucleotides.

In one embodiment the vaccine comprises an mRNA polynucleotide comprising an open reading frame encoding a coronavirus E protein or an immunogenic fragment thereof wherein the open reading frame comprises 150-400 nucleotides.

In one embodiment the vaccine comprises an mRNA polynucleotide comprising an open reading frame encoding a coronavirus E protein or an immunogenic fragment thereof wherein the open reading frame comprises 200-250 nucleotides.

In one embodiment the vaccine comprises an mRNA polynucleotide comprising an open reading frame encoding a coronavirus E protein or an immunogenic fragment thereof wherein the open reading frame consists of 228 nucleotides.

In one embodiment the vaccine comprises an mRNA polynucleotide comprising an open reading frame encoding a coronavirus M protein or an immunogenic fragment thereof wherein the open reading frame comprises 600-750 nucleotides.

In one embodiment the vaccine comprises an mRNA polynucleotide comprising an open reading frame encoding a coronavirus M protein or an immunogenic fragment thereof wherein the open reading frame comprises 650-700 nucleotides.

In one embodiment the vaccine comprises an mRNA polynucleotide comprising an open reading frame encoding a coronavirus M protein or an immunogenic fragment thereof wherein the open reading frame consists of 669 nucleotides.

In one embodiment the vaccine comprises an mRNA polynucleotide comprising an open reading frame encoding a coronavirus M, E, S, or N protein or an immunogenic fragment thereof.

In one embodiment the vaccine comprises an mRNA polynucleotide comprising an open reading frame encoding a coronavirus M, E, or S protein or an immunogenic fragment thereof.

In one embodiment the vaccine comprises an mRNA polynucleotide comprising an open reading frame encoding a coronavirus M protein or an immunogenic fragment thereof.

In one embodiment the vaccine comprises an mRNA polynucleotide comprising an open reading frame encoding a coronavirus E protein or an immunogenic fragment thereof.

In one embodiment the vaccine comprises an mRNA polynucleotide comprising an open reading frame encoding a coronavirus S protein or an immunogenic fragment thereof.

In one embodiment the vaccine comprises an mRNA polynucleotide comprising an open reading frame encoding a coronavirus N protein or an immunogenic fragment thereof.

In one embodiment the vaccine comprises an mRNA polynucleotide comprising an open reading frame encoding an alpha coronavirus antigen or an immunogenic fragment thereof.

In one embodiment the vaccine comprises an mRNA polynucleotide comprising an open reading frame encoding a beta coronavirus antigen or an immunogenic fragment thereof.

In one embodiment the vaccine comprises an mRNA polynucleotide comprising an open reading frame encoding a gamma coronavirus antigen or an immunogenic fragment thereof.

In one embodiment the vaccine comprises an mRNA polynucleotide comprising an open reading frame encoding a delta coronavirus antigen or an immunogenic fragment thereof.

In one embodiment the vaccine comprises an mRNA polynucleotide comprising an open reading frame encoding a 229E coronavirus antigen or an immunogenic fragment thereof.

In one embodiment the vaccine comprises an mRNA polynucleotide comprising an open reading frame encoding a NL63 coronavirus antigen or an immunogenic fragment thereof.

In one embodiment the vaccine comprises an mRNA polynucleotide comprising an open reading frame encoding a OC43 coronavirus antigen or an immunogenic fragment thereof.

In one embodiment the vaccine comprises an mRNA polynucleotide comprising an open reading frame encoding a HKU1 coronavirus antigen or an immunogenic fragment thereof.

In one embodiment the vaccine comprises an mRNA polynucleotide comprising an open reading frame encoding a MERS-CoV coronavirus antigen or an immunogenic fragment thereof.

In one embodiment the vaccine comprises an mRNA polynucleotide comprising an open reading frame encoding a SARS-CoV coronavirus antigen or an immunogenic fragment thereof.

In one embodiment the vaccine comprises an mRNA polynucleotide comprising an open reading frame encoding a SARS-CoV-2 coronavirus antigen or an immunogenic fragment thereof.

In one embodiment the mRNA polynucleotide comprising an open reading frame encoding a coronavirus antigen or an immunogenic fragment thereof encodes a SARS-CoV-2 coronavirus antigen or an immunogenic fragment thereof.

In one embodiment the vaccine comprises an mRNA polynucleotide comprising an open reading frame encoding a SARS-CoV-2 coronavirus M, E, S, or N protein or an immunogenic fragment thereof.

In one embodiment the vaccine comprises an mRNA polynucleotide comprising an open reading frame encoding a SARS-CoV-2 coronavirus M, S, or N protein or an immunogenic fragment thereof.

In one embodiment the vaccine comprises an mRNA polynucleotide comprising an open reading frame encoding a SARS-CoV-2 coronavirus M protein or an immunogenic fragment thereof.

In one embodiment the vaccine comprises an mRNA polynucleotide comprising an open reading frame encoding a SARS-CoV-2 coronavirus E protein or an immunogenic fragment thereof.

In one embodiment the vaccine comprises an mRNA polynucleotide comprising an open reading frame encoding a SARS-CoV-2 coronavirus S protein or an immunogenic fragment thereof.

In one embodiment the vaccine comprises an mRNA polynucleotide comprising an open reading frame encoding a SARS-CoV-2 coronavirus N protein or an immunogenic fragment thereof.

In one embodiment the vaccine comprises an mRNA polynucleotide comprising an open reading frame encoding original isolate SARS-CoV-2 coronavirus antigen or an immunogenic fragment thereof. Original isolate SARS-CoV-2 isolate was first identified in Wuhan, China in 2019.

In one embodiment the vaccine comprises an mRNA polynucleotide comprising an open reading frame encoding a SARS-CoV-2 coronavirus variant of concern antigen or an immunogenic fragment thereof. SARS-CoV-2 coronavirus variants of concern are identified and monitored by WHO. Variants of concern have been alpha, beta, delta, gamma, and omicron.

In one embodiment the vaccine comprises an mRNA polynucleotide comprising an open reading frame encoding a SARS-CoV-2 coronavirus alpha variant antigen or an immunogenic fragment thereof.

In one embodiment the vaccine comprises an mRNA polynucleotide comprising an open reading frame encoding a SARS-CoV-2 coronavirus beta variant antigen or an immunogenic fragment thereof.

In one embodiment the vaccine comprises an mRNA polynucleotide comprising an open reading frame encoding a SARS-CoV-2 coronavirus delta variant antigen or an immunogenic fragment thereof.

In one embodiment the vaccine comprises an mRNA polynucleotide comprising an open reading frame encoding a SARS-CoV-2 coronavirus gamma variant antigen or an immunogenic fragment thereof.

In one embodiment the vaccine comprises an mRNA polynucleotide comprising an open reading frame encoding a SARS-CoV-2 coronavirus omicron variant antigen or an immunogenic fragment thereof.

In one embodiment the vaccine comprises an mRNA polynucleotide comprising an open reading frame encoding a SARS-CoV-2 coronavirus variant under monitoring antigen or an immunogenic fragment thereof. SARS-CoV-2 coronavirus variants under monitoring are identified and monitored by WHO. From 15 Mar. 2023, the WHO variant tracking system considered the classification of Omicron sublineages independently as variants under monitoring. In May 2023 the WHO listed BA.2.75, CH.1.1, BQ.1, XBB, XBB.1.9.1, XBB.1.9.2 and XBF as variants under monitoring.

In one embodiment the vaccine comprises an mRNA polynucleotide comprising an open reading frame encoding a SARS-CoV-2 coronavirus variant of interest antigen or an immunogenic fragment thereof. SARS-CoV-2 coronavirus variants of interest are identified and monitored by WHO. Variants of interest have been epsilon, zeta, eta, theta, iota, kappa, lambda and mu. In May 2023 the WHO listed XBB.1.5 and XBB.1.16 as variants of interest.

In one embodiment the vaccine comprises an mRNA polynucleotide comprising an open reading frame encoding a SARS-CoV-2 coronavirus epsilon variant antigen or an immunogenic fragment thereof.

In one embodiment the vaccine comprises an mRNA polynucleotide comprising an open reading frame encoding a SARS-CoV-2 coronavirus zeta variant antigen or an immunogenic fragment thereof.

In one embodiment the vaccine comprises an mRNA polynucleotide comprising an open reading frame encoding a SARS-CoV-2 coronavirus eta variant antigen or an immunogenic fragment thereof.

In one embodiment the vaccine comprises an mRNA polynucleotide comprising an open reading frame encoding a SARS-CoV-2 coronavirus theta variant antigen or an immunogenic fragment thereof.

In one embodiment the vaccine comprises an mRNA polynucleotide comprising an open reading frame encoding a SARS-CoV-2 coronavirus iota variant antigen or an immunogenic fragment thereof.

In one embodiment the vaccine comprises an mRNA polynucleotide comprising an open reading frame encoding a SARS-CoV-2 coronavirus kappa variant antigen or an immunogenic fragment thereof.

In one embodiment the vaccine comprises an mRNA polynucleotide comprising an open reading frame encoding a SARS-CoV-2 coronavirus lambda variant antigen or an immunogenic fragment thereof.

In one embodiment the vaccine comprises an mRNA polynucleotide comprising an open reading frame encoding a SARS-CoV-2 coronavirus mu variant antigen or an immunogenic fragment thereof.

Table 1 describes certain mRNA open reading frame sequences that encode coronavirus antigens or immunogenic fragments thereof that may be incorporated into the mRNA polynucleotides in the vaccines described herein.

TABLE 1
mRNA polynucleotides encoding antigens

| Antigen | mRNA Open Reading Frame Sequence | |
|---|---|---|
| mRNA | (all uridines ″U″ are N1-methylpseudouridine nucleotides) | SEQ ID_No |
| SARS-CoV-2 | AUGUUUGUGUUCCUGGUUUUACUGCCACUAGUCUCCAGUCAGUGUGU | SEQ ID_No 2 |
| Spike | GAAUCUGACAACCAGAACCCAGCUCCCCCCUGCCUACACUAAUUCUUUC | |
| | ACACGUGGGGUUUAUUACCCUGACAAAGUUUUCCGGUCCUCAGUGUU | |
| | GCACAGCACCCAGGACUUGUUCCUCCCUUUCUUUUCCAAUGUGACUUG | |
| | GUUCCAUGCUAUACAUGUCUCUGGGACCAAUGGGACCAAGAGGUUUG | |
| | ACAACCCUGUCCUACCAUUCAAUGACGGUGUGUACUUUGCUUCCACCG | |
| | AGAAGUCUAACAUAAUCCGAGGCUGGAUUUUUGGUACCACUUUAGAC | |
| | UCGAAGACCCAGUCCCUACUUAUUGUGAAUAACGCCACUAAUGUGGUA | |
| | AUUAAAGUCUGUGAGUUUCAGUUUUGUAAUGAUCCAUUCUUGGGUG | |
| | UGUACUACCACAAGAACAACAAGAGUUGGAUGGAGAGUGAGUUCAGA | |
| | GUGUAUUCCAGCGCGAAUAACUGCACCUUCGAGUACGUCUCUCAGCCC | |
| | UUCCUUAUGGACCUCGAAGGAAAACAGGGUAACUUCAAGAAUCUGAG | |
| | GGAGUUCGUGUUUAAGAAUAUCGAUGGGUAUUUCAAGAUCUACUCUA | |
| | AGCACACGCCGAUUAAUCUGGUGAGAGAUCUCCCGCAGGGUUUCUCGG | |
| | CCUUGGAGCCACUGGUGGAUCUGCCAAUAGGGAUUAACAUCACUAGG | |
| | UUCCAAACGUUACUUGCUUUACACAGAAGUUAUCUAACCCCUGGGGAU | |
| | AGCUCAUCCGGGUGGACAGCUGGUGCGGCAGCUUAUUACGUGGGUUA | |
| | CCUGCAGCCUCGAACCUUCCUACUGAAAUACAACGAAAAUGGAACCAU | |
| | UACCGAUGCCGUAGACUGUGCACUUGACCCACUCUCAGAAACAAAGUG | |
| | CACGCUGAAGUCCUUCACCGUAGAGAAAGGAAUCUACCAGACUAGUAA | |
| | CUUCCGAGUCCAGCCGACAGAGUCUAUCGUUAGAUUUCCCAACAUCAC | |
| | AAACUUGUGCCCGUUUGGCGAAGUGUUCAACGCCACCCGGUUUGCAUC | |
| | UGUCUAUGCUUGGAACAGGAAGCGCAUCAGCAACUGUGUGGCCGAUU | |
| | ACAGCGUCCUAUACAACUCCGCCAGCUUUUCCACUUUUAAGUGUUAUG | |
| | GAGUGAGCCCUACUAAACUGAAUGACCUCUGCUUUACUAACGUCUAUG | |
| | CAGAUUCGUUCGUAAUCAGAGGCGAUGAGGUCAGACAGAUCGCCCCAG | |
| | GGCAAACUGGAAAGAUCGCGGACUAUAACUAUAAACUGCCAGACGAUU | |
| | UUACAGGCUGCGUGAUAGCUUGGAACUCUAACAAUCUUGAUUCUAAG | |
| | GUGGGGGGCAACUAUAAUUACCUGUAUCGAUUGUUUAGGAAGAGCAA | |
| | UCUCAAGCCUUUUGAGAGAGACAUUUCGACGGAGAUCUACCAGGCCGG | |
| | AAGCACACCUUGCAAUGGCGUUGAGGGGUUCAAUUGUUACUUCCCUC | |
| | UGCAGUCAUAUGGCUUCCAGCCCACUAAUGGCGUUGGAUACCAACCCU | |
| | ACCGAGUAGUAGUGCUUAGUUUUGAACUCCUGCACGCACCAGCCACCG | |
| | UUUGCGGACCCAAAAAGUCCACGAAUCUGGUGAAGAACAAGUGUGUCA | |
| | AUUUCAACUUCAAUGGGCUCACAGGCACAGGGGUUCUGACUGAGUCCA | |
| | ACAAGAAGUUUCUGCCUUUCCAGCAGUUCGGCCGCGACAUUGCUGACA | |
| | CUACUGAUGCUGUCCGCGACCCACAGACCUUGGAGAUUCUGGACAUCA | |
| | CCCCCUGCUCUUUCGGGGGCGUCAGCGUGAUAACCCCAGGAACAAAUA | |
| | CCUCCAACCAGGUGGCCGUGCUGUAUCAGGGGGUGAACUGCACAGAAG | |
| | UCCCUGUGGCUAUUCAUGCCGACCAACUUACUCCUACCUGGCGGGUGU | |
| | AUAGCACAGGCUCGAAUGUUUUCCAGACCCGGGCAGGCUGUUUGAUC | |
| | GGGGCUGAACAUGUCAACAACUCAUAUGAGUGUGACAUACCCAUCGGU | |
| | GCAGGAAUCUGCGCUAGCUACCAGACUCAGACUAACUCUCCCCGGCGG | |
| | GCACGUAGUGUAGCCAGUCAGUCCAUCAUUGCCUACACUAUGUCACUG | |
| | GGUGCCGAAAAUUCAGUGGCCUACUCUAACAACAGCAUUGCCAUACCC | |
| | ACAAAUUUUACGAUUAGUGUUACCACAGAAAUUCUACCAGUGUCUAU | |
| | GACCAAGACCUCAGUAGAUUGUACAAUGUACAUCUGUGGUGAUUCAA | |
| | CGGAAUGCAGCAAUCUGCUGCUGCAGUAUGGCAGUUUCUGCACACAGC | |
| | UGAACCGCGCUUUAACCGGAAUAGCCGUUGAGCAGGACAAAAACACCC | |
| | AGGAAGUUUUCGCACAGGUCAAGCAGAUCUACAAAACCCCCCCAAUCAA | |
| | AGACUUUGGAGGUUUCAACUUUAGCCAAAUACUCCCGGAUCCAUCAAA | |
| | GCCCAGCAAGAGGUCCUUUAUUGAAGAUCUACUCUUCAACAAAGUGAC | |
| | ACUGGCCGAUGCUGGCUUCAUCAAACAGUAUGGCGAUUGCCUUGGUG | |
| | AUAUCGCAGCGCGGGACCUCAUUUGCGCACAGAAGUUUAACGGCCUUA | |
| | CUGUGCUGCCACCCUUGCUCACCGACGAAAUGAUUGCUCAGUACACUU | |
| | CCGCACUGUUAGCGGGGACAAUCACGAGCGGGUGGACCUUUGGCGCAG | |
| | GCGCCGCCUUGCAGAUACCAUUCGCUAUGCAAAUGGCUUACAGGUUUA | |
| | AUGGGAUCGGAGUGACACAGAAUGUGCUCUAUGAGAACCAAAAACUGA | |
| | UCGCCAACCAAUUUAACAGUGCUAUCGGCAAAAUUCAAGACUCACUUU | |
| | CUUCCACAGCGAGUGCCCUUGGAAAACUGCAAGAUGUGGUCAACCAAA | |
| | AUGCACAGGCUCUGAACACGCUGGUUAAACAACUCAGCUCCAACUUUG | |
| | GAGCAAUUAGCAGUGUGCUGAAUGAUAUCCUUAGCCGUCUGGACCCCC | |
| | CCGAGGCGGAAGUGCAGAUUGAUAGGCUGAUCACAGGCAGACUUCAG | |
| | AGUCUGCAGACAUAUGUGACUCAACAGCUGAUUCGGGCCGCAGAAAUC | |
| | AGAGCCUCUGCCAAUCUGGCUGCUACUAAGAUGUCAGAGUGCGUCCUU | |
| | GGACAGUCAAAACGCGUGGAUUUUUGUGGAAAGGGCUACCACCUCAU | |
| | GUCCUUCCCUCAGUCAGCACCUCACGGCGUCGUCUUCUUGCAUGUGAC | |
| | AUACGUCCCCGCACAAGAGAAGAACUUCACAACUGCUCCUGCCAUCUGU | |
| | CACGAUGGAAAGGCACACUUCCCCCGCGAGGGUGUCUUUGUGUCCAAU | |
| | GGCACCCACUGGUUUGUAACACAAAGGAAUUUCUAUGAGCCCCAAAUC | |
| | AUCACUACGGACAACACAUUCGUGUCUGGGAACUGUGACGUGGUAAUC | |
| | GGAAUUGUCAACAACACAGUUUAUGACCCUCUGCAGCCUGAAUUGGAC | |
| | UCCUUCAAGGAGGAGUUAGAUAAAUACUUUAAGAACCAUACCAGCCCA | |
| | GACGUUGACCUCGGUGACAUCUCUGGCAUUAACGCCAGCGUUGUAAAC | |
| | AUUCAGAAAGAAAUUGACCGCCUCAAUGAGGUGGCCAAGAAUCUGAAU | |
| | GAAUCUCUCAUCGAUCUCCAGGAACUGGGAAAGUAUGAGCAGUACAUA | |
| | AAGUGGCCGUGGUACAUCUGGCUAGGUUUUAUCGCUGGCUUGAUUGC | |
| | CAUAGUAAUGGUGACAAUCAUGUUGUGCUGUAUGACCAGUUGCUGUA | |
| | GCUGCCUCAAGGGCUGUUGCAGCUGUGGAUCCUGCUGCAAAUUUGAU | |
| | GAAGACGACAGCGAGCCAGUGCUCAAAGGAGUCAAACUGCAUUACACC | |
| | UGA | |
| SARS-CoV-2 | AUGGCAGAUUCCAACGGCACCAUUACCGUCGAAGAGCUUAAAAAGCUC | SEQ ID_No 3 |
| Membrane | CUGGAACAAUGGAACCUAGUUAUAGGCUUCCUAUUCCUGACAUGGAU | |
| | UUGUCUGCUGCAGUUUGCCUAUGCCAACAGGAAUAGGUUUUUGUAUA | |
| | UAAUUAAGCUGAUUUUCCUCUGGCUGCUGUGGCCAGUAACUUUAGCU | |
| | UGUUUUGUGCUUGCUGCUGUGUACAGAAUAAAUUGGAUCACCGGGG | |
| | GAAUCGCAAUCGCAAUGGCUUGCCUUGUGGGCUUGAUGUGGCUCAGC | |
| | UACUUCAUCGCUUCUUUCAGACUGUUUGCGCGGACCCGAUCCAUGUG | |
| | GUCUUUCAAUCCCGAGACAAACAUCCUUCUCAACGUGCCCCUCCACGGC | |
| | ACUAUUCUGACCAGACCGUUACUCGAAUCGGAGCUCGUCAUCGGAGCC | |
| | GUGAUCCUGCGUGGACAUCUGCGGAUCGCCGGGCACCACCUAGGACGC | |
| | UGCGAUAUCAAGGACCUGCCUAAAGAGAUCACUGUGGCCACAUCACGA | |
| | ACGCUUUCUUAUUACAAAUUGGGGGCGAGCCAGCGGGUUGCAGGUGA | |
| | CUCAGGUUUUGCCGCAUACAGUCGCUACAGGAUUGGCAACUAUAAGCU | |
| | GAACACAGACCAUUCCAGCAGCAGUGACAAUAUUGCCUUGCUGGUCCA | |
| | GUGA | |
| SARS-CoV-2 | AUGUACUCAUUCGUAUCCGAAGAGACCGGCACGUUAAUCGUCAACAGC | SEQ ID_No 4 |
| Envelope | GUACUGCUUUUUUUGGCUUUCGUGGUCUUCCUGCUCGUGACACUAGC | |
| | CAUCCUCACUGCCCUUAGGCUGUGUGCAUACUGCUGCAAUAUUGUUA | |
| | ACGUGAGUCUGGUGAAACCAUCCUUUUAUGUGUACAGCCGGGUGAAG | |
| | AAUCUGAACAGCUCUAGAGUUCCCGACCUCCUGGUCUGA | |
| SARS-CoV-2 | AUGUUCGUGUUCCUCGUGCUGCUGCCCCUGGUGUCUAGCCAGUGCGU | SEQ ID_No 5 |
| SpikeZA | GAACCUGACCACCAGGACACAGCUGCCUCCAGCCUACACCAACAGCUUC | |
| (South | ACCAGGGGAGUGUACUACCCCGACAAGGUGUUCCGGAGCAGCGUGCUG | |
| African | CAUAGCACCCAGGACCUGUUCCUGCCCUUCUUCAGCAACGUGACUUGG | |
| B.1.351) | UUCCACGCCAUCCACGUCAGCGGAACAAACGGCACCAAGCGCUUCGCCA | |
| | ACCCAGUGCUGCCCUUCAACGACGGAGUGUACUUCGCCAGCACCGAGA | |
| | AGAGCAACAUCAUCCGGGGUUGGAUCUUCGGCACCACACUGGACAGCA | |
| | AGACCCAGAGCCUGCUGAUCGUGAACAACGCCACCAACGUCGUGAUCAA | |
| | GGUCUGCGAGUUCCAGUUUUGCAACGACCCCUUCCUGGGCGUGUACU | |
| | ACCACAAGAACAACAAGUCUUGGAUGGAGAGCGAGUUCCGGGUGUACA | |
| | GCAGCGCCAACAAUUGCACCUUCGAGUACGUGUCCCAGCCCUUCCUGA | |
| | UGGACCUGGAAGGCAAGCAGGGCAACUUCAAGAACCUGCGGGAGUUCG | |
| | UGUUCAAGAACAUCGACGGCUACUUCAAGAUCUACAGCAAGCACACCCC | |
| | CAUCAACCUCGUGAGAGGCCUGCCCCAGGGAUUCUCAGCUCUGGAACC | |
| | UCUGGUGGACCUGCCUAUCGGCAUCAACAUCACCCGGUUCCAGACCCU | |
| | GCAUAGGUCCUACCUGACCCCAGGAGACUCUUCUAGCGGCUGGACAGC | |
| | AGGAGCAGCCGCUUACUACGUGGGAUACCUGCAGCCUCGGACCUUCCU | |
| | GCUGAAGUACAACGAGAACGGCACCAUCACAGACGCCGUGGAUUGCGC | |
| | UCUGGAUCCUCUGAGCGAGACCAAGUGCACCCUGAAGAGCUUCACCGU | |
| | GGAGAAGGGCAUCUACCAGACCAGCAACUUCCGGGUGCAGCCUACAGA | |
| | GAGCAUCGUGCGGUUCCCUAACAUCACCAACCUCUGCCCCUUCGGCGA | |
| | GGUGUUCAACGCCACCAGAUUCGCCAGCGUGUACGCUUGGAACCGGAA | |
| | GCGGAUCAGCAACUGCGUGGCCGACUACAGCGUGCUGUACAACAGCGC | |
| | CAGCUUCAGCACCUUCAAGUGCUACGGCGUGUCCCCUACCAAGCUGAA | |
| | CGAUCUGUGCUUCACCAACGUGUACGCCGACAGCUUCGUGAUCAGGGG | |
| | AGACGAAGUGCGCCAGAUCGCUCCAGGACAGACAGGAAACAUCGCCGAC | |
| | UACAACUACAAGCUGCCCGACGACUUCACCGGUUGCGUGAUCGCUUGG | |
| | AACAGCAACAACCUGGACAGCAAGGUCGGCGGCAACUACAACUACCUGU | |
| | ACCGGCUGUUCCGGAAGAGCAACCUGAAGCCCUUCGAGCGGGACAUCA | |
| | GCACAGAGAUCUACCAGGCCGGCAGCACACCUUGUAACGGCGUGAAGG | |
| | GCUUCAACUGCUACUUCCCUCUGCAGAGCUACGGCUUCCAGCCUACAU | |
| | ACGGCGUGGGAUACCAGCCUUACAGGGUGGUGGUGCUGAGCUUCGAA | |
| | CUGCUGCACGCUCCAGCUACCGUCUGCGGACCUAAGAAGAGCACCAACC | |
| | UGGUGAAGAACAAGUGCGUGAAUUUCAACUUCAACGGCCUGACCGGAA | |
| | CCGGCGUGCUGACCGAGAGCAACAAGAAGUUCCUGCCCUUCCAGCAGU | |
| | UCGGCAGAGACAUCGCAGACACCACAGACGCCGUGAGAGAUCCUCAGAC | |
| | CCUGGAGAUCCUGGACAUCACCCCUUGCAGCUUCGGAGGAGUGUCCGU | |
| | GAUCACCCCAGGAACCAACACCAGCAAUCAGGUGGCCGUGCUGUACCAG | |
| | GGAGUGAAUUGCACCGAGGUGCCAGUGGCUAUUCACGCCGAUCAGCU | |
| | GACCCCUACUUGGAGAGUGUACAGCACCGGCAGCAACGUGUUCCAGAC | |
| | CAGAGCCGGUUGCCUGAUCGGAGCCGAGCACGUGAACAACAGCUACGA | |
| | GUGCGACAUCCCUAUCGGAGCCGGAAUCUGCGCUAGCUACCAGACCCA | |
| | GACCAACAGCCCUAGAAGAGCCAGAAGCGUGGCCUCUCAGAGCAUCAUC | |
| | GCCUACACCAUGAGCCUGGGAGUCGAAAACAGCGUGGCCUACAGCAAC | |
| | AACAGCAUCGCCAUCCCCACCAACUUCACCAUCAGCGUGACAACCGAGA | |
| | UCCUGCCAGUGUCCAUGACCAAGACCAGCGUCGACUGCACCAUGUACA | |
| | UCUGCGGCGACAGCACCGAGUGCUCUAACCUGCUGCUGCAGUACGGCU | |
| | CCUUUUGCACCCAGCUGAACAGGGCUCUGACAGGCAUCGCAGUGGAGC | |
| | AGGACAAGAACACCCAGGAGGUCUUCGCCCAGGUCAAGCAGAUCUACA | |
| | AGACCCCCCCUAUCAAGGACUUCGGAGGCUUCAACUUCAGCCAGAUCCU | |
| | GCCCGACCCUAGCAAGCCUAGCAAGCGGAGCUUCAUCGAGGACCUGCU | |
| | GUUCAACAAGGUCACCCUGGCCGACGCAGGCUUCAUCAAGCAGUACGG | |
| | CGAUUGCCUGGGCGACAUCGCCGCUAGAGACCUGAUUUGCGCCCAGAA | |
| | GUUCAACGGCCUGACAGUGCUGCCUCCUCUGCUGACCGACGAGAUGAU | |
| | CGCUCAGUACACAAGCGCUCUGCUGGCAGGAACCAUCACCAGCGGUUG | |
| | GACAUUCGGAGCCGGAGCCGCUCUGCAGAUCCCUUUUGCCAUGCAGAU | |
| | GGCCUACCGCUUCAACGGAAUCGGCGUGACCCAGAACGUGCUCUACGA | |
| | GAACCAGAAGCUGAUCGCCAACCAGUUCAACAGCGCCAUCGGCAAGAUC | |
| | CAGGACAGCCUGAGCAGCACAGCUAGCGCUCUGGGAAAGCUGCAGGAC | |
| | GUGGUGAACCAGAACGCCCAGGCUCUGAACACCCUGGUGAAGCAGCUG | |
| | AGCAGCAACUUCGGAGCCAUCAGCAGCGUGCUGAACGACAUCCUGAGC | |
| | AGACUGGACCCUCCAGAAGCCGAAGUGCAGAUCGACAGGCUGAUCACC | |
| | GGCAGACUGCAGUCUCUGCAGACCUACGUGACCCAGCAGCUGAUCAGG | |
| | GCCGCCGAAAUCAGAGCUAGCGCUAACCUGGCCGCCACAAAGAUGAGC | |
| | GAGUGCGUGCUGGGACAGAGCAAGAGAGUGGACUUCUGCGGCAAGGG | |
| | CUACCACCUGAUGAGCUUUCCUCAGAGCGCUCCUCACGGAGUGGUGUU | |
| | UCUGCACGUGACCUACGUGCCAGCCCAGGAGAAGAACUUCACCACCGCU | |
| | CCAGCCAUUUGCCACGACGGAAAGGCCCACUUCCCCAGAGAAGGCGUG | |
| | UUCGUGUCCAACGGCACCCAUUGGUUCGUGACCCAGCGGAACUUCUAC | |
| | GAGCCCCAGAUCAUCACCACCGACAACACCUUCGUGAGCGGCAAUUGCG | |
| | ACGUGGUCAUCGGCAUCGUGAACAACACCGUGUACGACCCCCUGCAGC | |
| | CAGAGCUGGACUCCUUCAAGGAGGAGCUGGACAAGUACUUCAAGAACC | |
| | ACACCAGCCCAGACGUGGACCUGGGAGAUAUCAGCGGCAUCAACGCCA | |
| | GCGUGGUGAACAUCCAGAAGGAGAUCGACAGGCUGAACGAGGUGGCCA | |
| | AGAACCUGAACGAGAGCCUGAUCGACCUGCAGGAGCUGGGCAAGUACG | |
| | AGCAGUACAUCAAGUGGCCUUGGUACAUUUGGCUGGGCUUCAUCGCC | |
| | GGCCUGAUCGCAAUCGUCAUGGUGACCAUCAUGCUCUGUUGCAUGACC | |
| | AGUUGCUGCAGCUGCCUGAAGGGUUGCUGCAGUUGCGGGAGUUGCUG | |
| | CAAGUUCGACGAGGACGACAGCGAGCCAGUGCUGAAGGGAGUGAAGCU | |
| | GCACUACACCUAG | |

In one embodiment the vaccine comprises an mRNA polynucleotide comprising an open reading frame comprising a sequence selected from SEQ ID_No 2 or a sequence with at least 80% sequence identity or homology.

In one embodiment the vaccine comprises an mRNA polynucleotide comprising an open reading frame comprising a sequence selected from SEQ ID_No 2 or a sequence with at least 90% sequence identity or homology.

In one embodiment the vaccine comprises an mRNA polynucleotide comprising an open reading frame comprising a sequence selected from SEQ ID_No 2 or a sequence with at least 95% sequence identity or homology.

In one embodiment the vaccine comprises an mRNA polynucleotide comprising an open reading frame comprising a sequence selected from SEQ ID_No 2 or a sequence with at least 98% sequence identity or homology.

In one embodiment the vaccine comprises an mRNA polynucleotide comprising an open reading frame comprising a sequence selected from SEQ ID_No 2.

In one embodiment the vaccine comprises an mRNA polynucleotide comprising an open reading frame consisting essentially of a sequence selected from SEQ ID_No 2.

In one embodiment the vaccine comprises an mRNA polynucleotide comprising an open reading frame consisting of a sequence selected from SEQ ID_No 2.

In one embodiment the vaccine comprises an mRNA polynucleotide comprising an open reading frame comprising a sequence selected from SEQ ID_No 3 or a sequence with at least 80% sequence identity or homology.

In one embodiment the vaccine comprises an mRNA polynucleotide comprising an open reading frame comprising a sequence selected from SEQ ID_No 3 or a sequence with at least 90% sequence identity or homology.

In one embodiment the vaccine comprises an mRNA polynucleotide comprising an open reading frame comprising a sequence selected from SEQ ID_No 3 or a sequence with at least 95% sequence identity or homology.

In one embodiment the vaccine comprises an mRNA polynucleotide comprising an open reading frame comprising a sequence selected from SEQ ID_No 3 or a sequence with at least 98% sequence identity or homology.

In one embodiment the vaccine comprises an mRNA polynucleotide comprising an open reading frame comprising a sequence selected from SEQ ID_No 3.

In one embodiment the vaccine comprises an mRNA polynucleotide comprising an open reading frame consisting essentially of a sequence selected from SEQ ID_No 3.

In one embodiment the vaccine comprises an mRNA polynucleotide comprising an open reading frame consisting of a sequence selected from SEQ ID_No 3.

In one embodiment the vaccine comprises an mRNA polynucleotide comprising an open reading frame comprising a sequence selected from SEQ ID_No 4 or a sequence with at least 80% sequence identity or homology.

In one embodiment the vaccine comprises an mRNA polynucleotide comprising an open reading frame comprising a sequence selected from SEQ ID_No 4 or a sequence with at least 90% sequence identity or homology.

In one embodiment the vaccine comprises an mRNA polynucleotide comprising an open reading frame comprising a sequence selected from SEQ ID_No 4 or a sequence with at least 95% sequence identity or homology.

In one embodiment the vaccine comprises an mRNA polynucleotide comprising an open reading frame comprising a sequence selected from SEQ ID_No 4 or a sequence with at least 98% sequence identity or homology.

In one embodiment the vaccine comprises an mRNA polynucleotide comprising an open reading frame comprising a sequence selected from SEQ ID_No 4.

In one embodiment the vaccine comprises an mRNA polynucleotide comprising an open reading frame consisting essentially of a sequence selected from SEQ ID_No 4.

In one embodiment the vaccine comprises an mRNA polynucleotide comprising an open reading frame consisting of a sequence selected from SEQ ID_No 4.

In one embodiment the vaccine comprises an mRNA polynucleotide comprising an open reading frame comprising a sequence selected from SEQ ID_No 5 or a sequence with at least 80% sequence identity or homology.

In one embodiment the vaccine comprises an mRNA polynucleotide comprising an open reading frame comprising a sequence selected from SEQ ID_No 5 or a sequence with at least 90% sequence identity or homology.

In one embodiment the vaccine comprises an mRNA polynucleotide comprising an open reading frame comprising a sequence selected from SEQ ID_No 5 or a sequence with at least 95% sequence identity or homology.

In one embodiment the vaccine comprises an mRNA polynucleotide comprising an open reading frame comprising a sequence selected from SEQ ID_No 5 or a sequence with at least 98% sequence identity or homology.

In one embodiment the vaccine comprises an mRNA polynucleotide comprising an open reading frame comprising a sequence selected from SEQ ID_No 5.

In one embodiment the vaccine comprises an mRNA polynucleotide comprising an open reading frame consisting essentially of a sequence selected from SEQ ID_No 5.

In one embodiment the vaccine comprises an mRNA polynucleotide comprising an open reading frame consisting of a sequence selected from SEQ ID_No 5.

### Encoding an Antigen

An antigen is a protein or a polypeptide from a coronavirus that binds to a specific antibody or T-cell receptor, triggering an immune response.

The antigens from a coronavirus encoded by the mRNA polynucleotide in the vaccines described herein can be “wild-type” i.e. naturally occurring antigens, or modified naturally occurring antigens. Non naturally occurring antigens that may be encoded by the mRNA polynucleotides in the vaccines herein are similar enough (e.g. 80% sequence identity or homology) to provoke the same immunogenic reaction as the equivalent wild-type antigen.

In one embodiment the vaccine comprises an mRNA polynucleotide comprising an open reading frame encoding a coronavirus antigen, or an immunogenic fragment thereof, wherein the antigen, or an immunogenic fragment thereof, has at least 80% sequence identity or homology with the wild-type antigen or fragment thereof.

In one embodiment the vaccine comprises an mRNA polynucleotide comprising an open reading frame encoding a coronavirus antigen, or an immunogenic fragment thereof, wherein the antigen, or an immunogenic fragment thereof, has at least 90% sequence identity or homology with the wild-type antigen or fragment thereof.

In one embodiment the vaccine comprises an mRNA polynucleotide comprising an open reading frame encoding a coronavirus antigen, or an immunogenic fragment thereof, wherein the antigen, or an immunogenic fragment thereof, has at least 95% sequence identity or homology with the wild-type antigen or fragment thereof.

In one embodiment the vaccine comprises an mRNA polynucleotide comprising an open reading frame encoding a coronavirus antigen, or an immunogenic fragment thereof, wherein the antigen, or an immunogenic fragment thereof, has at least 98% sequence identity or homology with the wild-type antigen or fragment thereof.

In one embodiment the vaccine comprises an mRNA polynucleotide comprising an open reading frame encoding a coronavirus antigen, or an immunogenic fragment thereof, wherein the antigen, or an immunogenic fragment thereof, is identical to the wild-type antigen or fragment thereof.

In one embodiment the vaccine comprises an mRNA polynucleotide comprising an open reading frame encoding a coronavirus antigen or an immunogenic fragment thereof wherein the antigen or immunogenic fragment thereof is a polypeptide.

In one embodiment the vaccine comprises an mRNA polynucleotide comprising an open reading frame encoding a coronavirus antigen or an immunogenic fragment thereof wherein the antigen or immunogenic fragment thereof is a polypeptide consisting of 50-1400 amino acid residues.

In one embodiment the vaccine comprises an mRNA polynucleotide comprising an open reading frame encoding a coronavirus antigen or an immunogenic fragment thereof wherein the antigen or immunogenic fragment thereof is a polypeptide consisting of 60-1300 amino acid residues.

In one embodiment the vaccine comprises an mRNA polynucleotide comprising an open reading frame encoding a coronavirus S protein or an immunogenic fragment thereof wherein the S protein or immunogenic fragment thereof is a polypeptide consisting of 1100-1400 amino acid residues.

In one embodiment the vaccine comprises an mRNA polynucleotide comprising an open reading frame encoding a coronavirus S protein or an immunogenic fragment thereof wherein the S protein or immunogenic fragment thereof is a polypeptide consisting of 1200-1300 amino acid residues.

In one embodiment the vaccine comprises an mRNA polynucleotide comprising an open reading frame encoding a coronavirus S protein or an immunogenic fragment thereof wherein the S protein or immunogenic fragment thereof is a polypeptide consisting of 1273 amino acid residues.

In one embodiment the vaccine comprises an mRNA polynucleotide comprising an open reading frame encoding a coronavirus S protein or an immunogenic fragment thereof wherein the S protein or immunogenic fragment thereof is a polypeptide consisting of 1270 amino acid residues.

In one embodiment the vaccine comprises an mRNA polynucleotide comprising an open reading frame encoding a coronavirus M protein or an immunogenic fragment thereof wherein the M protein or immunogenic fragment thereof is a polypeptide consisting of 150-300 amino acid residues.

In one embodiment the vaccine comprises an mRNA polynucleotide comprising an open reading frame encoding a coronavirus M protein or an immunogenic fragment thereof wherein the M protein or immunogenic fragment thereof is a polypeptide consisting of 200-250 amino acid residues.

In one embodiment the vaccine comprises an mRNA polynucleotide comprising an open reading frame encoding a coronavirus M protein or an immunogenic fragment thereof wherein the M protein or immunogenic fragment thereof is a polypeptide consisting of 222 amino acid residues.

In one embodiment the vaccine comprises an mRNA polynucleotide comprising an open reading frame encoding a coronavirus E protein or an immunogenic fragment thereof wherein the E protein or immunogenic fragment thereof is a polypeptide consisting of 50-100 amino acid residues.

In one embodiment the vaccine comprises an mRNA polynucleotide comprising an open reading frame encoding a coronavirus E protein or an immunogenic fragment thereof wherein the E protein or immunogenic fragment thereof is a polypeptide consisting of 70-80 amino acid residues.

In one embodiment the vaccine comprises an mRNA polynucleotide comprising an open reading frame encoding a coronavirus E protein or an immunogenic fragment thereof wherein the E protein or immunogenic fragment thereof is a polypeptide consisting of 75 amino acid residues.

In one embodiment the vaccine comprises an mRNA polynucleotide comprising an open reading frame encoding a coronavirus antigen or an immunogenic fragment thereof forming part or all of the coronavirus viral capsid.

In one embodiment the vaccine comprises an mRNA polynucleotide comprising an open reading frame encoding a coronavirus antigen that attaches to cell receptors, or an immunogenic fragment thereof.

In one embodiment the vaccine comprises an mRNA polynucleotide comprising an open reading frame encoding a coronavirus antigen that attaches to ACE2, or an immunogenic fragment thereof.

In one embodiment the vaccine comprises an mRNA polynucleotide comprising an open reading frame encoding a coronavirus antigen that causes fusion of viral and cellular membranes, or an immunogenic fragment thereof.

In one embodiment the vaccine comprises an mRNA polynucleotide comprising an open reading frame encoding a coronavirus antigen that is responsible for binding of the virus to a cell being infected, or an immunogenic fragment thereof.

Table 2 describes sequences of antigens from a SARS-CoV-2 coronavirus that the mRNA polynucleotides of the vaccines described herein may encode for:

TABLE 2
Antigens

| Antigen | Sequence | SEQ ID_No |
|---|---|---|
| SARS-CoV-2 | MFVFLVLLPLVSSQCVNLTTRTQLPPAYTNSFTRGVYYPDKVFRSSVLHSTQDL | SEQ ID_No 6 |
| Spike | FLPFFSNVTWFHAIHVSGTNGTKRFDNPVLPFNDGVYFASTEKSNIIRGWIFGT | |
| | TLDSKTQSLLIVNNATNVVIKVCEFQFCNDPFLGVYYHKNNKSWMESEFRVYS | |
| | SANNCTFEYVSQPFLMDLEGKQGNFKNLREFVFKNIDGYFKIYSKHTPINLVRD | |
| | LPQGFSALEPLVDLPIGINITRFQTLLALHRSYLTPGDSSSGWTAGAAAYYVGYL | |
| | QPRTFLLKYNENGTITDAVDCALDPLSETKCTLKSFTVEKGIYQTSNFRVQPTESI | |
| | VRFPNITNLCPFGEVFNATRFASVYAWNRKRISNCVADYSVLYNSASFSTFKCY | |
| | GVSPTKLNDLCFTNVYADSFVIRGDEVRQIAPGQTGKIADYNYKLPDDFTGCVI | |
| | AWNSNNLDSKVGGNYNYLYRLFRKSNLKPFERDISTEIYQAGSTPCNGVEGEN | |
| | CYFPLQSYGFQPTNGVGYQPYRVVVLSFELLHAPATVCGPKKSTNLVKNKCVN | |
| | FNFNGLTGTGVLTESNKKFLPFQQFGRDIADTTDAVRDPQTLEILDITPCSFGG | |
| | VSVITPGTNTSNQVAVLYQGVNCTEVPVAIHADQLTPTWRVYSTGSNVFQTR | |
| | AGCLIGAEHVNNSYECDIPIGAGICASYQTQTNSPRRARSVASQSIIAYTMSLG | |
| | AENSVAYSNNSIAIPTNFTISVTTEILPVSMTKTSVDCTMYICGDSTECSNLLLQY | |
| | GSFCTQLNRALTGIAVEQDKNTQEVFAQVKQIYKTPPIKDFGGFNFSQILPDPS | |
| | KPSKRSFIEDLLFNKVTLADAGFIKQYGDCLGDIAARDLICAQKFNGLTVLPPLLT | |
| | DEMIAQYTSALLAGTITSGWTFGAGAALQIPFAMQMAYRFNGIGVTQNVLYE | |
| | NQKLIANQFNSAIGKIQDSLSSTASALGKLQDVVNQNAQALNTLVKQLSSNFG | |
| | AISSVLNDILSRLDPPEAEVQIDRLITGRLQSLQTYVTQQLIRAAEIRASANLAAT | |
| | KMSECVLGQSKRVDFCGKGYHLMSFPQSAPHGVVFLHVTYVPAQEKNFTTAP | |
| | AICHDGKAHFPREGVFVSNGTHWFVTQRNFYEPQIITTDNTFVSGNCDVVIGI | |
| | VNNTVYDPLQPELDSFKEELDKYFKNHTSPDVDLGDISGINASVVNIQKEIDRL | |
| | NEVAKNLNESLIDLQELGKYEQYIKWPWYIWLGFIAGLIAIVMVTIMLCCMTSC | |
| | CSCLKGCCSCGSCCKFDEDDSEPVLKGVKLHYT | |
| SARS-CoV-2 | MADSNGTITVEELKKLLEQWNLVIGFLFLTWICLLQFAYANRNRFLYIIKLIFLWL | SEQ ID_No 7 |
| Membrane | LWPVTLACFVLAAVYRINWITGGIAIAMACLVGLMWLSYFIASFRLFARTRSM | |
| | WSFNPETNILLNVPLHGTILTRPLLESELVIGAVILRGHLRIAGHHLGRCDIKDLP | |
| | KEITVATSRTLSYYKLGASQRVAGDSGFAAYSRYRIGNYKLNTDHSSSSDNIALL | |
| | VQ | |
| SARS-CoV-2 | MYSFVSEETGTLIVNSVLLFLAFVVFLLVTLAILTALRLCAYCCNIVNVSLVKPSFY | SEQ ID_No 8 |
| Envelope | VYSRVKNLNSSRVPDLLV | |
| SARS-CoV-2 | MFVFLVLLPLVSSQCVNLTTRTQLPPAYTNSFTRGVYYPDKVFRSSVLHSTQDL | SEQ ID_No 9 |
| SpikeZA | FLPFFSNVTWFHAIHVSGTNGTKRFANPVLPFNDGVYFASTEKSNIIRGWIFGT | |
| (South | TLDSKTQSLLIVNNATNVVIKVCEFQFCNDPFLGVYYHKNNKSWMESEFRVYS | |
| African | SANNCTFEYVSQPFLMDLEGKQGNFKNLREFVFKNIDGYFKIYSKHTPINLVRG | |
| B.1.351) | LPQGFSALEPLVDLPIGINITRFQTLHRSYLTPGDSSSGWTAGAAAYYVGYLQP | |
| | RTFLLKYNENGTITDAVDCALDPLSETKCTLKSFTVEKGIYQTSNFRVQPTESIVR | |
| | FPNITNLCPFGEVFNATRFASVYAWNRKRISNCVADYSVLYNSASFSTFKCYGV | |
| | SPTKLNDLCFTNVYADSFVIRGDEVRQIAPGQTGNIADYNYKLPDDFTGCVIA | |
| | WNSNNLDSKVGGNYNYLYRLFRKSNLKPFERDISTEIYQAGSTPCNGVKGFNC | |
| | YFPLQSYGFQPTYGVGYQPYRVVVLSFELLHAPATVCGPKKSTNLVKNKCVNF | |
| | NFNGLTGTGVLTESNKKFLPFQQFGRDIADTTDAVRDPQTLEILDITPCSFGGV | |
| | SVITPGTNTSNQVAVLYQGVNCTEVPVAIHADQLTPTWRVYSTGSNVFQTRA | |
| | GCLIGAEHVNNSYECDIPIGAGICASYQTQTNSPRRARSVASQSIIAYTMSLGVE | |
| | NSVAYSNNSIAIPTNFTISVTTEILPVSMTKTSVDCTMYICGDSTECSNLLLQYGS | |
| | FCTQLNRALTGIAVEQDKNTQEVFAQVKQIYKTPPIKDFGGFNFSQILPDPSKP | |
| | SKRSFIEDLLFNKVTLADAGFIKQYGDCLGDIAARDLICAQKFNGLTVLPPLLTDE | |
| | MIAQYTSALLAGTITSGWTFGAGAALQIPFAMQMAYRFNGIGVTQNVLYEN | |
| | QKLIANQFNSAIGKIQDSLSSTASALGKLQDVVNQNAQALNTLVKQLSSNFGAI | |
| | SSVLNDILSRLDPPEAEVQIDRLITGRLQSLQTYVTQQLIRAAEIRASANLAATK | |
| | MSECVLGQSKRVDFCGKGYHLMSFPQSAPHGVVFLHVTYVPAQEKNFTTAP | |
| | AICHDGKAHFPREGVFVSNGTHWFVTQRNFYEPQIITTDNTFVSGNCDVVIGI | |
| | VNNTVYDPLQPELDSFKEELDKYFKNHTSPDVDLGDISGINASVVNIQKEIDRL | |
| | NEVAKNLNESLIDLQELGKYEQYIKWPWYIWLGFIAGLIAIVMVTIMLCCMTSC | |
| | CSCLKGCCSCGSCCKFDEDDSEPVLKGVKLHYT | |

In one embodiment the vaccine comprises an mRNA polynucleotide comprising an open reading frame encoding an antigen comprising a sequence selected from SEQ ID_No 6 or a sequence with at least 80% sequence identity or homology.

In one embodiment the vaccine comprises an mRNA polynucleotide comprising an open reading frame encoding an antigen comprising a sequence selected from SEQ ID_No 6 or a sequence with at least 90% sequence identity or homology.

In one embodiment the vaccine comprises an mRNA polynucleotide comprising an open reading frame encoding an antigen comprising a sequence selected from SEQ ID_No 6 or a sequence with at least 95% sequence identity or homology.

In one embodiment the vaccine comprises an mRNA polynucleotide comprising an open reading frame encoding an antigen comprising a sequence selected from SEQ ID_No 6 or a sequence with at least 98% sequence identity or homology.

In one embodiment the vaccine comprises an mRNA polynucleotide comprising an open reading frame encoding an antigen comprising a sequence selected from SEQ ID_No 6.

In one embodiment the vaccine comprises an mRNA polynucleotide comprising an open reading frame encoding an antigen consisting essentially of a sequence selected from SEQ ID_No 6.

In one embodiment the vaccine comprises an mRNA polynucleotide comprising an open reading frame encoding an antigen consisting of SEQ ID_No 6.

In one embodiment the vaccine comprises an mRNA polynucleotide comprising an open reading frame encoding an antigen comprising a sequence selected from SEQ ID_No 7 or a sequence with at least 80% sequence identity or homology.

In one embodiment the vaccine comprises an mRNA polynucleotide comprising an open reading frame encoding an antigen comprising a sequence selected from SEQ ID_No 7 or a sequence with at least 90% sequence identity or homology.

In one embodiment the vaccine comprises an mRNA polynucleotide comprising an open reading frame encoding an antigen comprising a sequence selected from SEQ ID_No 7 or a sequence with at least 95% sequence identity or homology.

In one embodiment the vaccine comprises an mRNA polynucleotide comprising an open reading frame encoding an antigen comprising a sequence selected from SEQ ID_No 7 or a sequence with at least 98% sequence identity or homology.

In one embodiment the vaccine comprises an mRNA polynucleotide comprising an open reading frame encoding an antigen comprising a sequence selected from SEQ ID_No 7.

In one embodiment the vaccine comprises an mRNA polynucleotide comprising an open reading frame encoding an antigen consisting essentially of a sequence selected from SEQ ID_No 7.

In one embodiment the vaccine comprises an mRNA polynucleotide comprising an open reading frame encoding an antigen consisting of SEQ ID_No 7.

In one embodiment the vaccine comprises an mRNA polynucleotide comprising an open reading frame encoding an antigen comprising a sequence selected from SEQ ID_No 8 or a sequence with at least 80% sequence identity or homology.

In one embodiment the vaccine comprises an mRNA polynucleotide comprising an open reading frame encoding an antigen comprising a sequence selected from SEQ ID_No 8 or a sequence with at least 90% sequence identity or homology.

In one embodiment the vaccine comprises an mRNA polynucleotide comprising an open reading frame encoding an antigen comprising a sequence selected from SEQ ID_No 8 or a sequence with at least 95% sequence identity or homology.

In one embodiment the vaccine comprises an mRNA polynucleotide comprising an open reading frame encoding an antigen comprising a sequence selected from SEQ ID_No 8 or a sequence with at least 98% sequence identity or homology.

In one embodiment the vaccine comprises an mRNA polynucleotide comprising an open reading frame encoding an antigen comprising a sequence selected from SEQ ID_No 8.

In one embodiment the vaccine comprises an mRNA polynucleotide comprising an open reading frame encoding an antigen consisting essentially of a sequence selected from SEQ ID_No 8.

In one embodiment the vaccine comprises an mRNA polynucleotide comprising an open reading frame encoding an antigen consisting of SEQ ID_No 8.

In one embodiment the vaccine comprises an mRNA polynucleotide comprising an open reading frame encoding an antigen comprising a sequence selected from SEQ ID_No 9 or a sequence with at least 90% sequence identity or homology.

In one embodiment the vaccine comprises an mRNA polynucleotide comprising an open reading frame encoding an antigen comprising a sequence selected from SEQ ID_No 9 or a sequence with at least 90% sequence identity or homology.

In one embodiment the vaccine comprises an mRNA polynucleotide comprising an open reading frame encoding an antigen comprising a sequence selected from SEQ ID_No 9 or a sequence with at least 95% sequence identity or homology.

In one embodiment the vaccine comprises an mRNA polynucleotide comprising an open reading frame encoding an antigen comprising a sequence selected from SEQ ID_No 9 or a sequence with at least 99% sequence identity or homology.

In one embodiment the vaccine comprises an mRNA polynucleotide comprising an open reading frame encoding an antigen comprising a sequence selected from SEQ ID_No 9.

In one embodiment the vaccine comprises an mRNA polynucleotide comprising an open reading frame encoding an antigen consisting essentially of a sequence selected from SEQ ID_No 9.

In one embodiment the vaccine comprises an mRNA polynucleotide comprising an open reading frame encoding an antigen consisting of SEQ ID_No 9.

Table 3 shows gene bank accession numbers for the sequences of certain coronavirus antigens from a coronavirus that the mRNA polynucleotides of the vaccines described herein may encode for:

TABLE 3
Gene bank accession numbers for certain coronavirus antigens

| | | GenBank/UniProtKB/ |
|---|---|---|
| Coronavirus | | Swiss-Prot |
| species | Coronavirus protein | Accession |
| SARS | Spike glycoprotein | YP_009825051.1 |
| coronavirus | Membrane glycoprotein M | YP_009825055.1 |
| Tor2 | small envelope protein | YP_009825054.1 |
| NC_004718.3 | Nucleocapsid protein | YP_009825061.1 |
| | NSP1 | YP_009944366.1 |
| | NSP2 | YP_009944367.1 |
| | NSP3 (papain-like proteinase) | YP_009944368.1 |
| | NSP4 | YP_009944369.1 |
| | NSP5 (3C-like protease) | YP_009944370.1 |
| | NSP6 | YP_009944371.1 |
| | NSP7 | YP_009944372.1 |
| | NSP8 | YP_009944373.1 |
| | NSP9 | YP_009944374.1 |
| | NSP10 | YP_009944375.1 |
| Middle East | Spike glycoprotein | YP_009047204.1 |
| respiratory | Membrane glycoprotein M | YP_009047210.1 |
| syndrome- | Envelope protein | YP_009047209.1 |
| related | Nucleocapsid protein | YP_009047211.1 |
| coronavirus | NSP1 | YP_009047213.1 |
| NC_019843.3 | NSP2 | YP_009047214.1 |
| | NSP3 (papain-like proteinase) | YP_009047215.1 |
| | NSP4 (putative transmembrane | YP_009047216.1 |
| | domain 2) | |
| | NSP5 (3C-like cysteine | YP_009047217.1 |
| | proteinase) | |
| | NSP6 (putative transmembrane | YP_009047218.1 |
| | protein 3) | |
| | NSP7 | YP_009047219.1 |
| | NSP8 (putative primase) | YP_009047220.1 |
| | NSP9 | YP_009047221.1 |
| | NSP10 | YP_009047222.1 |
| | NSP12 (RNA-dependent RNA | YP_009047223.1 |
| | polymerase) | |
| | NSP13 (putative zinc-binding | YP_009047224.1 |
| | domain, putative superfamily | |
| | 1 helicase) | |
| | NSP14 (N7-methyltransferase) | YP_009047225.1 |
| | NSP15 | YP_009047226.1 |
| | NSP16 (2′-O-methyltransferase) | YP_009047227.1 |
| Severe acute | Spike glycoprotein | YP_009724390.1 |
| respiratory | Membrane glycoprotein M | YP_009724393.1 |
| syndrome | Envelope protein | YP_009724392.1 |
| coronavirus | Nucleocapsid protein | YP_009724397.2 |
| 2 isolate | NSP1 | YP_009725297.1 |
| Wuhan-Hu-1 | NSP2 | YP_009725298.1 |
| NC_045512.2 | NSP3 (papain-like proteinase) | YP_009725299.1 |
| | NSP4 | YP_009725300.1 |
| | NSP5 (main protease) | YP_009725301.1 |
| | NSP6 (putative transmembrane | YP_009725302.1 |
| | protein) | |
| | NSP7 | YP_009725303.1 |
| | NSP8 (putative primase) | YP_009725304.1 |
| | NSP9 (ssRNA-binding protein) | YP_009725305.1 |
| | NSP10 | YP_009725306.1 |
| | NSP12 (RNA-dependent RNA | YP_009725307.1 |
| | polymerase) | |
| | NSP13 (putative zinc-binding | YP_009725308.1 |
| | domain, NTPase/helicase | |
| | domain) | |
| | NSP14 | YP_009725309.1 |
| | NSP15 | YP_009725310.1 |
| | NSP16 (2′-O-methyltransferase) | YP_009725311.1 |

In one embodiment the vaccine comprises an mRNA polynucleotide comprising an open reading frame encoding an antigen comprising a sequence selected from Table 3 or a sequence with at least 80% sequence identity or homology.

In one embodiment the vaccine comprises an mRNA polynucleotide comprising an open reading frame encoding an antigen comprising a sequence selected from Table 3 or a sequence with at least 90% sequence identity or homology.

In one embodiment the vaccine comprises an mRNA polynucleotide comprising an open reading frame encoding an antigen comprising a sequence selected from Table 3 or a sequence with at least 95% sequence identity or homology.

In one embodiment the vaccine comprises an mRNA polynucleotide comprising an open reading frame encoding an antigen comprising a sequence selected from Table 3 or a sequence with at least 98% sequence identity or homology.

In one embodiment the vaccine comprises an mRNA polynucleotide comprising an open reading frame encoding an antigen comprising a sequence selected from Table 3.

In one embodiment the vaccine comprises an mRNA polynucleotide comprising an open reading frame encoding an antigen consisting essentially of a sequence selected from Table 3.

In one embodiment the vaccine comprises an mRNA polynucleotide comprising an open reading frame encoding an antigen consisting of a sequence selected from Table 3.

### Immunogenic Fragment Thereof

An immunogenic fragment of an antigen (also known as an epitope, or antigenic determinant) is the fragment of the antigen that is recognized by the immune system, specifically by antibodies, B cells, or T cells, and induces an immune response. Immunogenic fragments are typically 8-15 amino acids long.

### Multiple Component Vaccines

In one embodiment the vaccine comprises multiple, e.g. 2-10, mRNA polynucleotides comprising open reading frames each encoding a single antigen from a coronavirus or an immunogenic fragment thereof. The two or more mRNA polynucleotides may encode the same or different antigens, or sequences from different parts of the same antigen, additionally or alternatively from different strains of coronavirus. In one embodiment the vaccine comprises 2, 3, 4, 5, 6, 7, 8, 9, or 10 mRNA polynucleotides comprising open reading frames each encoding a single antigen from a coronavirus or an immunogenic fragment thereof.

In one embodiment the vaccine comprises one mRNA polynucleotide comprising open reading frames encoding multiple, e.g. 2-10, antigens from a coronavirus or an immunogenic fragment thereof (e.g. as a fusion polypeptide). The open reading frames encoding multiple coronavirus antigens or an immunogenic fragment thereof may encode the same or different antigens, or sequences from different parts of the same antigen, additionally or alternatively from different strains of coronavirus. In one embodiment the vaccine comprises one mRNA polynucleotide comprising open reading frames encoding 2, 3, 4, 5, 6, 7, 8, 9 or 10 antigens from a coronavirus or an immunogenic fragment thereof.

In one embodiment, the vaccines described herein are multivalent. A multivalent vaccine combines antigens from different strains of coronavirus to immunize against one disease.

In one embodiment the vaccine comprises a first mRNA polynucleotide comprising an open reading frame encoding a coronavirus antigen or an immunogenic fragment thereof, and a second mRNA polynucleotide comprising an open reading frame encoding an antigen from a coronavirus or an immunogenic fragment thereof.

In one embodiment the vaccine comprises a first mRNA polynucleotide comprising an open reading frame encoding a coronavirus antigen or an immunogenic fragment thereof, a second mRNA polynucleotide comprising an open reading frame encoding an antigen from a coronavirus or an immunogenic fragment thereof, and a third mRNA polynucleotide comprising an open reading frame encoding an antigen from a coronavirus or an immunogenic fragment thereof.

In one embodiment the vaccine comprises one mRNA polynucleotide comprising open reading frames encoding two coronavirus antigens or immunogenic fragments thereof.

In one embodiment the vaccine comprises one mRNA polynucleotide comprising open reading frames encoding three coronavirus antigens or immunogenic fragments thereof.

In one embodiment the vaccine comprises:
i) a first mRNA polynucleotide comprising an open reading frame encoding a coronavirus antigen or an immunogenic fragment thereof;
ii) a second mRNA polynucleotide comprising an open reading frame encoding a coronavirus antigen or an immunogenic fragment thereof; and
iii) an amphipathic cell penetrating peptide comprising or consisting of the amino acid sequence WEARLARALARALARHLARALARALRACEA (SEQ ID_No 1) or a sequence with at least 80% sequence identity or homology.

In one embodiment the vaccine comprises:
i) a first mRNA polynucleotide comprising an open reading frame encoding a coronavirus antigen or an immunogenic fragment thereof;
ii) a second mRNA polynucleotide comprising an open reading frame encoding a coronavirus antigen or an immunogenic fragment thereof; and
iii) a third mRNA polynucleotide comprising an open reading frame encoding a coronavirus antigen or an immunogenic fragment thereof; and
iv) an amphipathic cell penetrating peptide comprising or consisting of the amino acid sequence WEARLARALARALARHLARALARALRACEA (SEQ ID_No 1) or a sequence with at least 80% sequence identity or homology.

In one embodiment the vaccine comprises three mRNA polynucleotides comprising an open reading frame each encoding a coronavirus antigen or an immunogenic fragment thereof wherein the three mRNA polynucleotides are present in an equal % w/w ratio.

In one embodiment the vaccine comprises three mRNA polynucleotides comprising an open reading frame each encoding a coronavirus antigen or an immunogenic fragment thereof wherein the three mRNA polynucleotides are present in an equal copy number ratio.

“Copy number” refers to the number of copies of the mRNA present. To determine copy numbers of mRNA, the molecular weight (MW) of one copy of mRNA is calculated by addition of the molecular weights of all constituent nucleotides in the entire nucleotide sequence, where:

$$A = 329.2 \text{g/} mol \\ C = 305.2 \text{g/} mol \\ G = 345.2 \text{g/} mol \\ T = 306.2 \text{g/} mol$$

Any proprietary sequences (non-ORF), for which the precise nucleotides are not known, are accounted for by using the average molecular weight of A/G/C/T=321.45 g/mol.

The copy numbers present in a known mass of mRNA (m) are then calculated as follows:

$$Copy Number = ( m / M W ) * NA$$

(where NA=Avogadro's Number=6.0221409×10\hat{ }23)

In one embodiment the vaccine comprises three mRNA polynucleotides comprising an open reading frame each encoding a coronavirus antigen or an immunogenic fragment thereof wherein the three mRNA polynucleotides are present in an unequal % w/w ratio.

In one embodiment the vaccine comprises three mRNA polynucleotides comprising an open reading frame each encoding a coronavirus antigen or an immunogenic fragment thereof wherein the three mRNA polynucleotides are present in unequal copy number ratios.

In one embodiment the vaccine comprises two mRNA polynucleotides comprising an open reading frame each encoding a coronavirus antigen or an immunogenic fragment thereof wherein the two mRNA polynucleotides are present in a 50:50% w/w ratio.

In one embodiment the vaccine comprises two mRNA polynucleotides comprising an open reading frame each encoding a coronavirus antigen or an immunogenic fragment thereof wherein the two mRNA polynucleotides are present in a 1:1 copy number ratio.

In one embodiment the vaccine comprises two mRNA polynucleotides comprising an open reading frame each encoding a coronavirus antigen or an immunogenic fragment thereof wherein the two mRNA polynucleotides are present in about a 50:50% w/w ratio.

In one embodiment the vaccine comprises two mRNA polynucleotides comprising an open reading frame each encoding a coronavirus antigen or an immunogenic fragment thereof wherein the two mRNA polynucleotides are present in about a 1:1 copy number ratio.

In one embodiment the vaccine comprises two mRNA polynucleotides comprising open reading frames each encoding a different coronavirus M, E, S, or N protein or an immunogenic fragment thereof.

In one embodiment the vaccine comprises two mRNA polynucleotides comprising open reading frames each encoding the same M, E, S, or N protein or an immunogenic fragment thereof, wherein each protein or fragment thereof is from a different variant.

In one embodiment the vaccine comprises three mRNA polynucleotides comprising open reading frames each encoding a different coronavirus M, E, S, or N protein or an immunogenic fragment thereof.

In one embodiment the vaccine comprises three mRNA polynucleotides comprising open reading frames each encoding the same M, E, S, or N protein or an immunogenic fragment thereof, wherein each protein or fragment thereof is from a different variant.

In one embodiment the vaccine comprises two mRNA polynucleotides comprising open reading frames each encoding a different coronavirus M, E, or S protein or an immunogenic fragment thereof.

In one embodiment the vaccine comprises two mRNA polynucleotides comprising open reading frames each encoding the same M, E or S protein or an immunogenic fragment thereof, wherein each protein or fragment thereof is from a different variant.

In one embodiment the vaccine comprises three mRNA polynucleotides comprising open reading frames each encoding a different coronavirus M, E, or S protein or an immunogenic fragment thereof.

In one embodiment the vaccine comprises three mRNA polynucleotides comprising open reading frames each encoding the same M, E or S protein or an immunogenic fragment thereof, wherein each protein or fragment thereof is from a different variant.

In one embodiment the vaccine comprises three mRNA polynucleotides comprising open reading frames one encoding an M coronavirus protein or an immunogenic fragment thereof, one encoding an E coronavirus protein or an immunogenic fragment thereof, and one encoding an S coronavirus protein or an immunogenic fragment thereof.

In one embodiment the vaccine comprises two mRNA polynucleotides comprising open reading frames one encoding an S coronavirus protein or an immunogenic fragment thereof, and one encoding an M coronavirus protein or an immunogenic fragment thereof.

In one embodiment the vaccine comprises two mRNA polynucleotides comprising open reading frames one encoding an S coronavirus protein or an immunogenic fragment thereof, and one encoding an E coronavirus protein or an immunogenic fragment thereof.

In one embodiment the vaccine comprises two or more mRNA polynucleotides comprising open reading frames each encoding an M coronavirus protein or an immunogenic fragment thereof, wherein each M protein or fragment thereof is from a different variant.

In one embodiment the vaccine comprises two or more mRNA polynucleotides comprising open reading frames each encoding an E coronavirus protein or an immunogenic fragment thereof, wherein each E protein or fragment thereof is from a different variant.

In one embodiment the vaccine comprises two or more mRNA polynucleotides comprising open reading frames each encoding an S coronavirus protein or an immunogenic fragment thereof, wherein each S protein or fragment thereof is from a different variant.

In one embodiment the vaccine comprises two or more mRNA polynucleotides comprising open reading frames each encoding an N coronavirus protein or an immunogenic fragment thereof, wherein each N protein or fragment thereof is from a different variant.

In one embodiment the vaccine comprises two mRNA polynucleotides comprising open reading frames each encoding a different SARS-CoV-2 M, E, S, or N protein or an immunogenic fragment thereof.

In one embodiment the vaccine comprises two mRNA polynucleotides comprising open reading frames each encoding the same SARS-CoV-2 M, E, S, or N protein or an immunogenic fragment thereof, wherein each protein or fragment thereof is from a different variant.

In one embodiment the vaccine comprises three mRNA polynucleotides comprising open reading frames each encoding a different SARS-CoV-2 M, E, S, or N protein or an immunogenic fragment thereof.

In one embodiment the vaccine comprises three mRNA polynucleotides comprising open reading frames each encoding the same SARS-CoV-2 M, E, S, or N protein or an immunogenic fragment thereof, wherein each protein or fragment thereof is from a different variant.

In one embodiment the vaccine comprises two mRNA polynucleotides comprising open reading frames each encoding a different SARS-CoV-2 M, E, or S protein or an immunogenic fragment thereof.

In one embodiment the vaccine comprises two mRNA polynucleotides comprising open reading frames each encoding the same SARS-CoV-2 M, E or S protein or an immunogenic fragment thereof, wherein each protein or fragment thereof is from a different variant.

In one embodiment the vaccine comprises three mRNA polynucleotides comprising open reading frames each encoding a different SARS-CoV-2 M, E, or S protein or an immunogenic fragment thereof.

In one embodiment the vaccine comprises three mRNA polynucleotides comprising open reading frames each encoding the same SARS-CoV-2 M, E or S protein or an immunogenic fragment thereof, wherein each protein or fragment thereof is from a different variant.

In one embodiment the vaccine comprises three mRNA polynucleotides comprising open reading frames one encoding an M SARS-CoV-2 protein or an immunogenic fragment thereof, one encoding an E SARS-CoV-2 protein or an immunogenic fragment thereof, and one encoding an S SARS-CoV-2 protein or an immunogenic fragment thereof.

In one embodiment the vaccine comprises two mRNA polynucleotides comprising open reading frames one encoding an S SARS-CoV-2 protein or an immunogenic fragment thereof, and one encoding an E SARS-CoV-2 protein or an immunogenic fragment thereof.

In one embodiment the vaccine comprises two mRNA polynucleotides comprising open reading frames one encoding an S SARS-CoV-2 protein or an immunogenic fragment thereof, and one encoding an M SARS-CoV-2 protein or an immunogenic fragment thereof.

In one embodiment the vaccine comprises two or more mRNA polynucleotides comprising open reading frames each encoding an M SARS-CoV-2 coronavirus protein or an immunogenic fragment thereof, wherein each M protein or fragment thereof is from a different variant.

In one embodiment the vaccine comprises two or more mRNA polynucleotides comprising open reading frames each encoding an E SARS-CoV-2 coronavirus protein or an immunogenic fragment thereof, wherein each E protein or fragment thereof is from a different variant.

In one embodiment the vaccine comprises two or more mRNA polynucleotides comprising open reading frames each encoding an S SARS-CoV-2 coronavirus protein or an immunogenic fragment thereof, wherein each S protein or fragment thereof is from a different variant.

In one embodiment the vaccine comprises two or more mRNA polynucleotides comprising open reading frames each encoding an N SARS-CoV-2 coronavirus protein or an immunogenic fragment thereof, wherein each N protein or fragment thereof is from a different variant.

In one embodiment the vaccine comprises three mRNA polynucleotides comprising open reading frames one encoding an M SARS-CoV-2 protein or an immunogenic fragment thereof, one encoding an E SARS-CoV-2 protein or an immunogenic fragment thereof, and one encoding an S SARS-CoV-2 protein or an immunogenic fragment thereof wherein the three mRNA polynucleotides are present in an equal % w/w ratio.

In one embodiment the vaccine comprises three mRNA polynucleotides comprising open reading frames one encoding an M SARS-CoV-2 protein or an immunogenic fragment thereof, one encoding an E SARS-CoV-2 protein or an immunogenic fragment thereof, and one encoding an S SARS-CoV-2 protein or an immunogenic fragment thereof wherein the three mRNA polynucleotides are present in a 1:1:1 copy number ratio.

In one embodiment the vaccine comprises three mRNA polynucleotides comprising open reading frames one encoding an M SARS-CoV-2 protein or an immunogenic fragment thereof, one encoding an E SARS-CoV-2 protein or an immunogenic fragment thereof, and one encoding an S SARS-CoV-2 protein or an immunogenic fragment thereof wherein the three mRNA polynucleotides are present in about an equal % w/w ratio.

In one embodiment the vaccine comprises three mRNA polynucleotides comprising open reading frames one encoding an M SARS-CoV-2 protein or an immunogenic fragment thereof, one encoding an E SARS-CoV-2 protein or an immunogenic fragment thereof, and one encoding an S SARS-CoV-2 protein or an immunogenic fragment thereof wherein the three mRNA polynucleotides are present in about a 1:1:1 copy number ratio.

In one embodiment the vaccine comprises three mRNA polynucleotides comprising open reading frames one encoding an M SARS-CoV-2 protein or an immunogenic fragment thereof, one encoding an E SARS-CoV-2 protein or an immunogenic fragment thereof, and one encoding an S SARS-CoV-2 protein or an immunogenic fragment thereof wherein the three mRNA polynucleotides are present in an unequal % w/w ratio.

In one embodiment the vaccine comprises three mRNA polynucleotides comprising open reading frames one encoding an M SARS-CoV-2 protein or an immunogenic fragment thereof, one encoding an E SARS-CoV-2 protein or an immunogenic fragment thereof, and one encoding an S SARS-CoV-2 protein or an immunogenic fragment thereof wherein the three mRNA polynucleotides are present in an unequal copy number ratio.

In one embodiment the vaccine comprises two mRNA polynucleotides comprising open reading frames each encoding a different SARS-CoV-2 M, E, S, or N protein or an immunogenic fragment thereof wherein the M, E, S, or N proteins or immunogenic fragments thereof are from the same SARS-CoV-2 variant.

In one embodiment the vaccine comprises three mRNA polynucleotides comprising open reading frames each encoding a different SARS-CoV-2 M, E, S, or N protein or an immunogenic fragment thereof wherein the M, E, S, or N proteins or immunogenic fragments thereof are from the same SARS-CoV-2 variant.

In one embodiment the vaccine comprises two mRNA polynucleotides comprising open reading frames each encoding a different SARS-CoV-2 M, E, or S protein or an immunogenic fragment thereof wherein the M, E, or S proteins or immunogenic fragments thereof are from the same SARS-CoV-2 variant.

In one embodiment the vaccine comprises three mRNA polynucleotides comprising open reading frames each encoding a different SARS-CoV-2 M, E, or S protein or an immunogenic fragment thereof wherein the M, E or S proteins or immunogenic fragments thereof are from the same SARS-CoV-2 variant.

In one embodiment the vaccine comprises three mRNA polynucleotides comprising open reading frames one encoding an M SARS-CoV-2 protein or an immunogenic fragment thereof, one encoding an E SARS-CoV-2 protein or an immunogenic fragment thereof, and one encoding an S SARS-CoV-2 protein or an immunogenic fragment thereof wherein the M, E, or S proteins or immunogenic fragments thereof are from the same SARS-CoV-2 variant.

In one embodiment the vaccine comprises three mRNA polynucleotides comprising open reading frames one encoding an M SARS-CoV-2 protein or an immunogenic fragment thereof, one encoding an E SARS-CoV-2 protein or an immunogenic fragment thereof, and one encoding an S SARS-CoV-2 protein or an immunogenic fragment thereof wherein the M, E, or S proteins or immunogenic fragments thereof are from the same SARS-CoV-2 variant wherein the three mRNA polynucleotides are present in an equal % w/w ratio.

In one embodiment the vaccine comprises three mRNA polynucleotides comprising open reading frames one encoding an M SARS-CoV-2 protein or an immunogenic fragment thereof, one encoding an E SARS-CoV-2 protein or an immunogenic fragment thereof, and one encoding an S SARS-CoV-2 protein or an immunogenic fragment thereof wherein the M, E, or S proteins or immunogenic fragments thereof are from the same SARS-CoV-2 variant wherein the three mRNA polynucleotides are present in a 1:1:1 copy number ratio.

In one embodiment the vaccine comprises three mRNA polynucleotides comprising open reading frames one encoding an M SARS-CoV-2 protein or an immunogenic fragment thereof, one encoding an E SARS-CoV-2 protein or an immunogenic fragment thereof, and one encoding an S SARS-CoV-2 protein or an immunogenic fragment thereof wherein the M, E, or S proteins or immunogenic fragments thereof are from the same SARS-CoV-2 variant wherein the three mRNA polynucleotides are present in an about equal % w/w ratio.

In one embodiment the vaccine comprises three mRNA polynucleotides comprising open reading frames one encoding an M SARS-CoV-2 protein or an immunogenic fragment thereof, one encoding an E SARS-CoV-2 protein or an immunogenic fragment thereof, and one encoding an S SARS-CoV-2 protein or an immunogenic fragment thereof wherein the M, E, or S proteins or immunogenic fragments thereof are from the same SARS-CoV-2 variant wherein the three mRNA polynucleotides are present in about a 1:1:1 copy number ratio.

In one embodiment the vaccine comprises three mRNA polynucleotides comprising open reading frames one encoding an M SARS-CoV-2 protein or an immunogenic fragment thereof, one encoding an E SARS-CoV-2 protein or an immunogenic fragment thereof, and one encoding an S SARS-CoV-2 protein or an immunogenic fragment thereof wherein the M, E, or S proteins or immunogenic fragments thereof are from the same SARS-CoV-2 variant wherein the three mRNA polynucleotides are present in an unequal % w/w ratio.

In one embodiment the vaccine comprises three mRNA polynucleotides comprising open reading frames one encoding an M SARS-CoV-2 protein or an immunogenic fragment thereof, one encoding an E SARS-CoV-2 protein or an immunogenic fragment thereof, and one encoding an S SARS-CoV-2 protein or an immunogenic fragment thereof wherein the M, E, or S proteins or immunogenic fragments thereof are from the same SARS-CoV-2 variant wherein the three mRNA polynucleotides are present in an unequal copy number ratio.

In one embodiment the vaccine comprises two mRNA polynucleotides comprising open reading frames each encoding a different SARS-CoV-2 M, E, S, or N protein or an immunogenic fragment thereof wherein the M, E, S, or N proteins or immunogenic fragments thereof are from different SARS-CoV-2 variants.

In one embodiment the vaccine comprises three mRNA polynucleotides comprising open reading frames each encoding a different SARS-CoV-2 M, E, S, or N protein or an immunogenic fragment thereof wherein the M, E, S, or N proteins or immunogenic fragments thereof are from different SARS-CoV-2 variants.

In one embodiment the vaccine comprises two mRNA polynucleotides comprising open reading frames each encoding a different SARS-CoV-2 M, E, or S protein or an immunogenic fragment thereof wherein the M, E, or S proteins or immunogenic fragments thereof are from different SARS-CoV-2 variants.

In one embodiment the vaccine comprises three mRNA polynucleotides comprising open reading frames each encoding a different SARS-CoV-2 M, E, or S protein or an immunogenic fragment thereof wherein the M, E or S proteins or immunogenic fragments thereof are from different SARS-CoV-2 variants.

In one embodiment the vaccine comprises three mRNA polynucleotides comprising open reading frames one encoding an M SARS-CoV-2 protein or an immunogenic fragment thereof, one encoding an E SARS-CoV-2 protein or an immunogenic fragment thereof, and one encoding an S SARS-CoV-2 protein or an immunogenic fragment thereof wherein the M, E, or S proteins or immunogenic fragments thereof are from different SARS-CoV-2 variants.

In one embodiment the vaccine comprises three mRNA polynucleotides comprising open reading frames one encoding an M SARS-CoV-2 protein or an immunogenic fragment thereof, one encoding an E SARS-CoV-2 protein or an immunogenic fragment thereof, and one encoding an S SARS-CoV-2 protein or an immunogenic fragment thereof wherein the M, E, or S proteins or immunogenic fragments thereof are from different SARS-CoV-2 variants wherein the three mRNA polynucleotides are present in an equal % w/w ratio.

In one embodiment the vaccine comprises three mRNA polynucleotides comprising open reading frames one encoding an M SARS-CoV-2 protein or an immunogenic fragment thereof, one encoding an E SARS-CoV-2 protein or an immunogenic fragment thereof, and one encoding an S SARS-CoV-2 protein or an immunogenic fragment thereof wherein the M, E, or S proteins or immunogenic fragments thereof are from different SARS-CoV-2 variants wherein the three mRNA polynucleotides are present in a 1:1:1 copy number ratio.

In one embodiment the vaccine comprises three mRNA polynucleotides comprising open reading frames one encoding an M SARS-CoV-2 protein or an immunogenic fragment thereof, one encoding an E SARS-CoV-2 protein or an immunogenic fragment thereof, and one encoding an S SARS-CoV-2 protein or an immunogenic fragment thereof wherein the M, E, or S proteins or immunogenic fragments thereof are from different SARS-CoV-2 variants wherein the three mRNA polynucleotides are present in an about equal % w/w ratio.

In one embodiment the vaccine comprises three mRNA polynucleotides comprising open reading frames one encoding an M SARS-CoV-2 protein or an immunogenic fragment thereof, one encoding an E SARS-CoV-2 protein or an immunogenic fragment thereof, and one encoding an S SARS-CoV-2 protein or an immunogenic fragment thereof wherein the M, E, or S proteins or immunogenic fragments thereof are from different SARS-CoV-2 variants wherein the three mRNA polynucleotides are present in about a 1:1:1 copy number ratio.

In one embodiment the vaccine comprises three mRNA polynucleotides comprising open reading frames one encoding an M SARS-CoV-2 protein or an immunogenic fragment thereof, one encoding an E SARS-CoV-2 protein or an immunogenic fragment thereof, and one encoding an S SARS-CoV-2 protein or an immunogenic fragment thereof wherein the M, E, or S proteins or immunogenic fragments thereof are from different SARS-CoV-2 variants wherein the three mRNA polynucleotides are present in an unequal % w/w ratio.

In one embodiment the vaccine comprises three mRNA polynucleotides comprising open reading frames one encoding an M SARS-CoV-2 protein or an immunogenic fragment thereof, one encoding an E SARS-CoV-2 protein or an immunogenic fragment thereof, and one encoding an S SARS-CoV-2 protein or an immunogenic fragment thereof wherein the M, E, or S proteins or immunogenic fragments thereof are from different SARS-CoV-2 variants wherein the three mRNA polynucleotides are present in an unequal copy number ratio.

In one embodiment the vaccine comprises an mRNA polynucleotide comprising an open reading frame comprising a sequence selected from SEQ ID_No 2 or a sequence with at least 80% sequence identity or homology, and an mRNA polynucleotide comprising an open reading frame comprising a sequence selected from SEQ ID_No 3 or a sequence with at least 80% sequence identity or homology, and an mRNA polynucleotide comprising an open reading frame comprising a sequence selected from SEQ ID_No 4 or a sequence with at least 80% sequence identity or homology.

In one embodiment the vaccine comprises an mRNA polynucleotide comprising an open reading frame comprising a sequence selected from SEQ ID_No 5 or a sequence with at least 80% sequence identity or homology, and an mRNA polynucleotide comprising an open reading frame comprising a sequence selected from SEQ ID_No 3 or a sequence with at least 80% sequence identity or homology, and an mRNA polynucleotide comprising an open reading frame comprising a sequence selected from SEQ ID_No 4 or a sequence with at least 80% sequence identity or homology.

In one embodiment the vaccine comprises an mRNA polynucleotide comprising an open reading frame comprising a sequence selected from SEQ ID_No 2 or a sequence with at least 90% sequence identity or homology, and an mRNA polynucleotide comprising an open reading frame comprising a sequence selected from SEQ ID_No 3 or a sequence with at least 90% sequence identity or homology, and an mRNA polynucleotide comprising an open reading frame comprising a sequence selected from SEQ ID_No 4 or a sequence with at least 90% sequence identity or homology.

In one embodiment the vaccine comprises an mRNA polynucleotide comprising an open reading frame comprising a sequence selected from SEQ ID_No 5 or a sequence with at least 90% sequence identity or homology, and an mRNA polynucleotide comprising an open reading frame comprising a sequence selected from SEQ ID_No 3 or a sequence with at least 90% sequence identity or homology, and an mRNA polynucleotide comprising an open reading frame comprising a sequence selected from SEQ ID_No 4 or a sequence with at least 90% sequence identity or homology.

In one embodiment the vaccine comprises an mRNA polynucleotide comprising an open reading frame comprising a sequence selected from SEQ ID_No 2 or a sequence with at least 95% sequence identity or homology, and an mRNA polynucleotide comprising an open reading frame comprising a sequence selected from SEQ ID_No 3 or a sequence with at least 95% sequence identity or homology, and an mRNA polynucleotide comprising an open reading frame comprising a sequence selected from SEQ ID_No 4 or a sequence with at least 95% sequence identity or homology.

In one embodiment the vaccine comprises an mRNA polynucleotide comprising an open reading frame comprising a sequence selected from SEQ ID_No 5 or a sequence with at least 95% sequence identity or homology, and an mRNA polynucleotide comprising an open reading frame comprising a sequence selected from SEQ ID_No 3 or a sequence with at least 95% sequence identity or homology, and an mRNA polynucleotide comprising an open reading frame comprising a sequence selected from SEQ ID_No 4 or a sequence with at least 95% sequence identity or homology.

In one embodiment the vaccine comprises an mRNA polynucleotide comprising an open reading frame comprising a sequence selected from SEQ ID_No 2 or a sequence with at least 80% sequence identity or homology and an mRNA polynucleotide comprising an open reading frame comprising a sequence selected from SEQ ID_No 3 or a sequence with at least 80% sequence identity or homology, and an mRNA polynucleotide comprising an open reading frame comprising a sequence selected from SEQ ID_No 4 or a sequence with at least 80% sequence identity or homology wherein the three mRNA polynucleotides are present in an equal % w/w ratio.

In one embodiment the vaccine comprises an mRNA polynucleotide comprising an open reading frame comprising a sequence selected from SEQ ID_No 5 or a sequence with at least 80% sequence identity or homology and an mRNA polynucleotide comprising an open reading frame comprising a sequence selected from SEQ ID_No 3 or a sequence with at least 80% sequence identity or homology, and an mRNA polynucleotide comprising an open reading frame comprising a sequence selected from SEQ ID_No 4 or a sequence with at least 80% sequence identity or homology wherein the three mRNA polynucleotides are present in an equal % w/w ratio.

In one embodiment the vaccine comprises an mRNA polynucleotide comprising an open reading frame comprising a sequence selected from SEQ ID_No 2 or a sequence with at least 80% sequence identity or homology and an mRNA polynucleotide comprising an open reading frame comprising a sequence selected from SEQ ID_No 3 or a sequence with at least 80% sequence identity or homology, and an mRNA polynucleotide comprising an open reading frame comprising a sequence selected from SEQ ID_No 4 or a sequence with at least 80% sequence identity or homology wherein the three mRNA polynucleotides are present in a 1:1:1 copy number ratio.

In one embodiment the vaccine comprises an mRNA polynucleotide comprising an open reading frame comprising a sequence selected from SEQ ID_No 5 or a sequence with at least 80% sequence identity or homology and an mRNA polynucleotide comprising an open reading frame comprising a sequence selected from SEQ ID_No 3 or a sequence with at least 80% sequence identity or homology, and an mRNA polynucleotide comprising an open reading frame comprising a sequence selected from SEQ ID_No 4 or a sequence with at least 80% sequence identity or homology wherein the three mRNA polynucleotides are present in a 1:1:1 copy number ratio.

In one embodiment the vaccine comprises an mRNA polynucleotide comprising an open reading frame comprising a sequence selected from SEQ ID_No 2 or a sequence with at least 80% sequence identity or homology and an mRNA polynucleotide comprising an open reading frame comprising a sequence selected from SEQ ID_No 3 or a sequence with at least 80% sequence identity or homology, and an mRNA polynucleotide comprising an open reading frame comprising a sequence selected from SEQ ID_No 4 or a sequence with at least 80% sequence identity or homology wherein the three mRNA polynucleotides are present in about an equal % w/w ratio.

In one embodiment the vaccine comprises an mRNA polynucleotide comprising an open reading frame comprising a sequence selected from SEQ ID_No 5 or a sequence with at least 80% sequence identity or homology and an mRNA polynucleotide comprising an open reading frame comprising a sequence selected from SEQ ID_No 3 or a sequence with at least 80% sequence identity or homology, and an mRNA polynucleotide comprising an open reading frame comprising a sequence selected from SEQ ID_No 4 or a sequence with at least 80% sequence identity or homology wherein the three mRNA polynucleotides are present in about an equal % w/w ratio.

In one embodiment the vaccine comprises an mRNA polynucleotide comprising an open reading frame comprising a sequence selected from SEQ ID_No 2 or a sequence with at least 80% sequence identity or homology and an mRNA polynucleotide comprising an open reading frame comprising a sequence selected from SEQ ID_No 3 or a sequence with at least 80% sequence identity or homology, and an mRNA polynucleotide comprising an open reading frame comprising a sequence selected from SEQ ID_No 4 or a sequence with at least 80% sequence identity or homology wherein the three mRNA polynucleotides are present in about a 1:1:1 copy number ratio.

In one embodiment the vaccine comprises an mRNA polynucleotide comprising an open reading frame comprising a sequence selected from SEQ ID_No 5 or a sequence with at least 80% sequence identity or homology and an mRNA polynucleotide comprising an open reading frame comprising a sequence selected from SEQ ID_No 3 or a sequence with at least 80% sequence identity or homology, and an mRNA polynucleotide comprising an open reading frame comprising a sequence selected from SEQ ID_No 4 or a sequence with at least 80% sequence identity or homology wherein the three mRNA polynucleotides are present in about a 1:1:1 copy number ratio.

In one embodiment the vaccine comprises an mRNA polynucleotide comprising an open reading frame comprising a sequence selected from SEQ ID_No 2 or a sequence with at least 80% sequence identity or homology and an mRNA polynucleotide comprising an open reading frame comprising a sequence selected from SEQ ID_No 3 or a sequence with at least 80% sequence identity or homology, and an mRNA polynucleotide comprising an open reading frame comprising a sequence selected from SEQ ID_No 4 or a sequence with at least 80% sequence identity or homology wherein the three mRNA polynucleotides are present in an unequal % w/w ratio.

In one embodiment the vaccine comprises an mRNA polynucleotide comprising an open reading frame comprising a sequence selected from SEQ ID_No 5 or a sequence with at least 80% sequence identity or homology and an mRNA polynucleotide comprising an open reading frame comprising a sequence selected from SEQ ID_No 3 or a sequence with at least 80% sequence identity or homology, and an mRNA polynucleotide comprising an open reading frame comprising a sequence selected from SEQ ID_No 4 or a sequence with at least 80% sequence identity or homology wherein the three mRNA polynucleotides are present in an unequal % w/w ratio.

In one embodiment the vaccine comprises an mRNA polynucleotide comprising an open reading frame comprising a sequence selected from SEQ ID_No 2 or a sequence with at least 80% sequence identity or homology and an mRNA polynucleotide comprising an open reading frame comprising a sequence selected from SEQ ID_No 3 or a sequence with at least 80% sequence identity or homology, and an mRNA polynucleotide comprising an open reading frame comprising a sequence selected from SEQ ID_No 4 or a sequence with at least 80% sequence identity or homology wherein the three mRNA polynucleotides are present in an unequal copy number ratio.

In one embodiment the vaccine comprises an mRNA polynucleotide comprising an open reading frame comprising a sequence selected from SEQ ID_No 5 or a sequence with at least 80% sequence identity or homology and an mRNA polynucleotide comprising an open reading frame comprising a sequence selected from SEQ ID_No 3 or a sequence with at least 80% sequence identity or homology, and an mRNA polynucleotide comprising an open reading frame comprising a sequence selected from SEQ ID_No 4 or a sequence with at least 80% sequence identity or homology wherein the three mRNA polynucleotides are present in an unequal copy number ratio.

In one embodiment the vaccine comprises an mRNA polynucleotide comprising an open reading frame comprising a sequence selected from SEQ ID_No 2 or a sequence with at least 80% sequence identity or homology and an mRNA polynucleotide comprising an open reading frame comprising a sequence selected from SEQ ID_No 3 or a sequence with at least 80% sequence identity or homology, and an mRNA polynucleotide comprising an open reading frame comprising a sequence selected from SEQ ID_No 4 or a sequence with at least 80% sequence identity or homology wherein the three mRNA polynucleotides are present in an about equal % w/w ratio.

In one embodiment the vaccine comprises an mRNA polynucleotide comprising an open reading frame comprising a sequence selected from SEQ ID_No 5 or a sequence with at least 80% sequence identity or homology and an mRNA polynucleotide comprising an open reading frame comprising a sequence selected from SEQ ID_No 3 or a sequence with at least 80% sequence identity or homology, and an mRNA polynucleotide comprising an open reading frame comprising a sequence selected from SEQ ID_No 4 or a sequence with at least 80% sequence identity or homology wherein the three mRNA polynucleotides are present in an about equal % w/w ratio.

In one embodiment the vaccine comprises an mRNA polynucleotide comprising an open reading frame comprising a sequence selected from SEQ ID_No 2 or a sequence with at least 80% sequence identity or homology and an mRNA polynucleotide comprising an open reading frame comprising a sequence selected from SEQ ID_No 3 or a sequence with at least 80% sequence identity or homology, and an mRNA polynucleotide comprising an open reading frame comprising a sequence selected from SEQ ID_No 4 or a sequence with at least 80% sequence identity or homology wherein the three mRNA polynucleotides are present in an about a 1:1:1 copy number ratio.

In one embodiment the vaccine comprises an mRNA polynucleotide comprising an open reading frame comprising a sequence selected from SEQ ID_No 5 or a sequence with at least 80% sequence identity or homology and an mRNA polynucleotide comprising an open reading frame comprising a sequence selected from SEQ ID_No 3 or a sequence with at least 80% sequence identity or homology, and an mRNA polynucleotide comprising an open reading frame comprising a sequence selected from SEQ ID_No 4 or a sequence with at least 80% sequence identity or homology wherein the three mRNA polynucleotides are present in an about a 1:1:1 copy number ratio.

In one embodiment the vaccine comprises an mRNA polynucleotide comprising an open reading frame comprising a sequence selected from SEQ ID_No 2, and an mRNA polynucleotide comprising an open reading frame comprising a sequence selected from SEQ ID_No 3, and an mRNA polynucleotide comprising an open reading frame comprising a sequence selected from SEQ ID_No 4, wherein the three mRNA polynucleotides are present in an equal % w/w ratio.

In one embodiment the vaccine comprises an mRNA polynucleotide comprising an open reading frame comprising a sequence selected from SEQ ID_No 5, and an mRNA polynucleotide comprising an open reading frame comprising a sequence selected from SEQ ID_No 3, and an mRNA polynucleotide comprising an open reading frame comprising a sequence selected from SEQ ID_No 4, wherein the three mRNA polynucleotides are present in an equal % w/w ratio.

In one embodiment the vaccine comprises an mRNA polynucleotide comprising an open reading frame comprising a sequence selected from SEQ ID_No 2, and an mRNA polynucleotide comprising an open reading frame comprising a sequence selected from SEQ ID_No 3, and an mRNA polynucleotide comprising an open reading frame comprising a sequence selected from SEQ ID_No 4, wherein the three mRNA polynucleotides are present in a 1:1:1 copy number ratio.

In one embodiment the vaccine comprises an mRNA polynucleotide comprising an open reading frame comprising a sequence selected from SEQ ID_No 5, and an mRNA polynucleotide comprising an open reading frame comprising a sequence selected from SEQ ID_No 3, and an mRNA polynucleotide comprising an open reading frame comprising a sequence selected from SEQ ID_No 4, wherein the three mRNA polynucleotides are present in a 1:1:1 copy number ratio.

In one embodiment the vaccine comprises an mRNA polynucleotide comprising an open reading frame comprising a sequence selected from SEQ ID_No 2, and an mRNA polynucleotide comprising an open reading frame comprising a sequence selected from SEQ ID_No 3, and an mRNA polynucleotide comprising an open reading frame comprising a sequence selected from SEQ ID_No 4, wherein the three mRNA polynucleotides are present in an unequal % w/w ratio.

In one embodiment the vaccine comprises an mRNA polynucleotide comprising an open reading frame comprising a sequence selected from SEQ ID_No 5, and an mRNA polynucleotide comprising an open reading frame comprising a sequence selected from SEQ ID_No 3, and an mRNA polynucleotide comprising an open reading frame comprising a sequence selected from SEQ ID_No 4, wherein the three mRNA polynucleotides are present in an unequal % w/w ratio.

In one embodiment the vaccine comprises an mRNA polynucleotide comprising an open reading frame comprising a sequence selected from SEQ ID_No 2, and an mRNA polynucleotide comprising an open reading frame comprising a sequence selected from SEQ ID_No 3, and an mRNA polynucleotide comprising an open reading frame comprising a sequence selected from SEQ ID_No 4, wherein the three mRNA polynucleotides are present in an unequal copy number ratio.

In one embodiment the vaccine comprises an mRNA polynucleotide comprising an open reading frame comprising a sequence selected from SEQ ID_No 5, and an mRNA polynucleotide comprising an open reading frame comprising a sequence selected from SEQ ID_No 3, and an mRNA polynucleotide comprising an open reading frame comprising a sequence selected from SEQ ID_No 4, wherein the three mRNA polynucleotides are present in an unequal copy number ratio.

In one embodiment the vaccine comprises an mRNA polynucleotide comprising an open reading frame comprising a sequence selected from SEQ ID_No 2, and an mRNA polynucleotide comprising an open reading frame comprising a sequence selected from SEQ ID_No 3, and an mRNA polynucleotide comprising an open reading frame comprising a sequence selected from SEQ ID_No 4, wherein the three mRNA polynucleotides are present in about an equal % w/w ratio.

In one embodiment the vaccine comprises an mRNA polynucleotide comprising an open reading frame comprising a sequence selected from SEQ ID_No 5, and an mRNA polynucleotide comprising an open reading frame comprising a sequence selected from SEQ ID_No 3, and an mRNA polynucleotide comprising an open reading frame comprising a sequence selected from SEQ ID_No 4, wherein the three mRNA polynucleotides are present in about an equal % w/w ratio.

In one embodiment the vaccine comprises an mRNA polynucleotide comprising an open reading frame comprising a sequence selected from SEQ ID_No 2, and an mRNA polynucleotide comprising an open reading frame comprising a sequence selected from SEQ ID_No 3, and an mRNA polynucleotide comprising an open reading frame comprising a sequence selected from SEQ ID_No 4, wherein the three mRNA polynucleotides are present in about a 1:1:1 copy number ratio.

In one embodiment the vaccine comprises an mRNA polynucleotide comprising an open reading frame comprising a sequence selected from SEQ ID_No 5, and an mRNA polynucleotide comprising an open reading frame comprising a sequence selected from SEQ ID_No 3, and an mRNA polynucleotide comprising an open reading frame comprising a sequence selected from SEQ ID_No 4, wherein the three mRNA polynucleotides are present in about a 1:1:1 copy number ratio.

In one embodiment the vaccine comprises a first mRNA polynucleotide comprising an open reading frame encoding an antigen selected from SEQ ID_No 6 or a sequence with at least 80% sequence identity or homology; a second mRNA polynucleotide comprising an open reading frame encoding an antigen selected from SEQ ID_No 7 or a sequence with at least 80% sequence identity or homology; a third mRNA polynucleotide comprising an open reading frame encoding an antigen selected from SEQ ID_No 8 or a sequence with at least 80% sequence identity or homology.

In one embodiment the vaccine comprises a first mRNA polynucleotide comprising an open reading frame encoding an antigen selected from SEQ ID_No 9 or a sequence with at least 80% sequence identity or homology; a second mRNA polynucleotide comprising an open reading frame encoding an antigen selected from SEQ ID_No 7 or a sequence with at least 80% sequence identity or homology; a third mRNA polynucleotide comprising an open reading frame encoding an antigen selected from SEQ ID_No 8 or a sequence with at least 80% sequence identity or homology.

In one embodiment the vaccine comprises a first mRNA polynucleotide comprising an open reading frame encoding an antigen selected from SEQ ID_No 6 or a sequence with at least 90% sequence identity or homology; a second mRNA polynucleotide comprising an open reading frame encoding an antigen selected from SEQ ID_No 7 or a sequence with at least 90% sequence identity or homology; a third mRNA polynucleotide comprising an open reading frame encoding an antigen selected from SEQ ID_No 8 or a sequence with at least 90% sequence identity or homology.

In one embodiment the vaccine comprises a first mRNA polynucleotide comprising an open reading frame encoding an antigen selected from SEQ ID_No 9 or a sequence with at least 90% sequence identity or homology; a second mRNA polynucleotide comprising an open reading frame encoding an antigen selected from SEQ ID_No 7 or a sequence with at least 90% sequence identity or homology; a third mRNA polynucleotide comprising an open reading frame encoding an antigen selected from SEQ ID_No 8 or a sequence with at least 90% sequence identity or homology.

In one embodiment the vaccine comprises a first mRNA polynucleotide comprising an open reading frame encoding an antigen selected from SEQ ID_No 6 or a sequence with at least 95% sequence identity or homology; a second mRNA polynucleotide comprising an open reading frame encoding an antigen selected from SEQ ID_No 7 or a sequence with at least 95% sequence identity or homology; a third mRNA polynucleotide comprising an open reading frame encoding an antigen selected from SEQ ID_No 8 or a sequence with at least 95% sequence identity or homology.

In one embodiment the vaccine comprises a first mRNA polynucleotide comprising an open reading frame encoding an antigen selected from SEQ ID_No 9 or a sequence with at least 95% sequence identity or homology; a second mRNA polynucleotide comprising an open reading frame encoding an antigen selected from SEQ ID_No 7 or a sequence with at least 95% sequence identity or homology; a third mRNA polynucleotide comprising an open reading frame encoding an antigen selected from SEQ ID_No 8 or a sequence with at least 95% sequence identity or homology.

### Immunoadjuvants

In one embodiment the vaccine further comprises an immunoadjuvant. An immunoadjuvant is a substance that acts to accelerate, prolong, or enhance an antigen-specific immune response when used in combination with specific vaccine antigens.

Suitable immunoadjuvants include inorganic compounds, for example potassium alum KAI(SO₄)₂, aluminium hydroxide, aluminium phosphate, and calcium phosphate hydroxide; oils, for example paraffin oil, propolis, and peanut oil; and cytokines, for example IL-1 (interleukin-1), IL-2, IL-7, IL-12, IL-15 and granulocyte-macrophage colony-stimulating factor (GM-CSF).

In one embodiment the vaccine further comprises an mRNA polynucleotide comprising an open reading frame encoding an immunoadjuvant. In one embodiment the vaccine comprises an mRNA polynucleotide comprising an open reading frame encoding a coronavirus antigen or an immunogenic fragment thereof, and an open reading frame encoding an immunoadjuvant.

In one embodiment the vaccine further comprises an mRNA polynucleotide comprising an open reading frame encoding a cytokine.

In one embodiment the vaccine further comprises an mRNA polynucleotide comprising an open reading frame encoding IL-1, IL-2, IL-7, IL-12 or IL-15.

In one embodiment the vaccine further comprises an mRNA polynucleotide comprising an open reading frame encoding IL-15.

In one embodiment the vaccine further comprises an mRNA polynucleotide comprising an open reading frame encoding human IL-15.

In one embodiment the vaccine further comprises an mRNA polynucleotide comprising an open reading frame encoding granulocyte-macrophage colony-stimulating factor.

In one embodiment the vaccine further comprises an mRNA polynucleotide comprising an open reading frame encoding human granulocyte-macrophage colony-stimulating factor.

Table 4 describes certain mRNA open reading frame sequences encoding immunoadjuvants that may be incorporated mRNA polynucleotides and added to the vaccines described herein.

TABLE 4
mRNA polynucleotides encoding immunoadjuvants

| | mRNA Open Reading Frame Sequence | |
|---|---|---|
| Immunoadjuvant | (all uridines ″U″ are N1-methylpseudouridine nucleotides) | SEQ ID_No |
| hIL-15 (human | AUGAGAAUCAGCAAGCCGCACCUGAGAAGCAUCAGCAUUCAGU | SEQ ID_No 10 |
| interleukin 15) | GCUACCUCUGCCUGCUGCUCAACAGCCACUUCCUGACCGAAGC | |
| | CGGAAUCCACGUAUUCAUCCUGGGAUGCUUCAGCGCCGGCCUC | |
| | CCCAAAACAGAGGCAAACUGGGUAAACGUAAUAAGCGACCUCA | |
| | AAAAGAUCGAAGACCUGAUCCAGAGCAUGCACAUCGACGCAAC | |
| | CCUGUACACCGAAAGCGACGUACACCCAAGCUGCAAAGUAACC | |
| | GCGAUGAAAUGCUUCCUGCUGGAGCUGCAAGUAAUCAGCCUG | |
| | GAGAGCGGCGACGCCAGCAUCCACGACACCGUGGAAAACCUGA | |
| | UUAUCUUGGCAAACAACAGCCUGAGCAGCAACGGAAACGUGAC | |
| | AGAGAGCGGAUGCAAAGAGUGCGAGGAGCUGGAGGAAAAGAA | |
| | CAUCAAAGAAUUCCUCCAAAGCUUCGUGCACAUAGUGCAGAUG | |
| | UUCAUAAACACAAGCUGA | |
| hGM-CSF | AUGUGGCUGCAAAGCCUUCUGCUCUUGGGGACUGUGGCCUGC | SEQ ID_No 11 |
| | UCAAUUUCUGCACCUGCCCGAUCGCCCAGUCCAAGCACGCAGC | |
| | CCUGGGAGCAUGUGAAUGCCAUCCAGGAGGCGCGGAGGUUAC | |
| | UGAACCUGAGUAGAGACACUGCUGCUGAGAUGAAUGAAACAG | |
| | UAGAAGUGAUAUCAGAAAUGUUUGAUCUCCAGGAGCCUACCU | |
| | GCCUACAGACACGCCUGGAGCUGUAUAAACAGGGCCUGAGGG | |
| | GCUCCCUCACCAAGCUCAAAGGACCCUUGACAAUGAUGGCCAG | |
| | CCACUACAAGCAACACUGUCCUCCAACCCCGGAAACUUCCUGU | |
| | GCAACCCAGAUUAUCACAUUUGAAUCCUUCAAAGAGAACCUGA | |
| | AGGAUUUCCUACUUGUUAUCCCCUUCGACUGCUGGGAGCCAG | |
| | UCCAGGAGUGA | |

In one embodiment the vaccine further comprises an mRNA polynucleotide comprising an open reading frame encoding an immunoadjuvant comprising a sequence selected from SEQ ID_No 10 or a sequence with at least 80% sequence identity or homology.

In one embodiment the vaccine further comprises an mRNA polynucleotide comprising an open reading frame encoding an immunoadjuvant comprising a sequence selected from SEQ ID_No 10 or a sequence with at least 90% sequence identity or homology.

In one embodiment the vaccine further comprises an mRNA polynucleotide comprising an open reading frame encoding an immunoadjuvant comprising a sequence selected from SEQ ID_No 10 or a sequence with at least 95% sequence identity or homology.

In one embodiment the vaccine further comprises an mRNA polynucleotide comprising an open reading frame encoding an immunoadjuvant comprising a sequence selected from SEQ ID_No 10 or a sequence with at least 98% sequence identity or homology.

In one embodiment the vaccine further comprises an mRNA polynucleotide comprising an open reading frame encoding an immunoadjuvant comprising a sequence selected from SEQ ID_No 10.

In one embodiment there is provided an mRNA polynucleotide comprising an open reading frame encoding an immunoadjuvant comprising a sequence selected from SEQ ID_No 10.

In one embodiment the vaccine further comprises an mRNA polynucleotide comprising an open reading frame encoding an immunoadjuvant comprising a sequence selected from SEQ ID_No 11 or a sequence with at least 80% sequence identity or homology.

In one embodiment the vaccine further comprises an mRNA polynucleotide comprising an open reading frame encoding an immunoadjuvant comprising a sequence selected from SEQ ID_No 11 or a sequence with at least 90% sequence identity or homology.

In one embodiment the vaccine further comprises an mRNA polynucleotide comprising an open reading frame encoding an immunoadjuvant comprising a sequence selected from SEQ ID_No 11 or a sequence with at least 95% sequence identity or homology.

In one embodiment the vaccine further comprises an mRNA polynucleotide comprising an open reading frame encoding an immunoadjuvant comprising a sequence selected from SEQ ID_No 11 or a sequence with at least 98% sequence identity or homology.

In one embodiment the vaccine further comprises an mRNA polynucleotide comprising an open reading frame encoding an immunoadjuvant comprising a sequence selected from SEQ ID_No 11.

In one embodiment there is provided an mRNA polynucleotide comprising an open reading frame encoding an immunoadjuvant comprising a sequence selected from SEQ ID_No 11.

Table 5 describes sequences of immunoadjuvant that the mRNA polynucleotides of the vaccines described herein may encode for:

TABLE 5
Immunoadjuvant

| Immunoadjuvant | Sequence | SEQ ID_No |
|---|---|---|
| Human IL-15 | MRISKPHLRSISIQCYLCLLLNSHFLTEAGIHVFILGCFSAGLPKTEANW | SEQ ID_No 12 |
| | VNVISDLKKIEDLIQSMHIDATLYTESDVHPSCKVTAMKCFLLELQVIS | |
| | LESGDASIHDTVENLIILANNSLSSNGNVTESGCKECEELEEKNIKEFLQ | |
| | SFVHIVQMFINTS | |
| Human GM-CSF | MWLQSLLLLGTVACSISAPARSPSPSTQPWEHVNAIQEARRLLNLSR | SEQ ID_No 13 |
| | DTAAEMNETVEVISEMFDLQEPTCLQTRLELYKQGLRGSLTKLKGPLT | |
| | MMASHYKQHCPPTPETSCATQIITFESFKENLKDFLLVIPFDCWE | |
| | PVQE | |

In one embodiment the vaccine further comprises an mRNA polynucleotide comprising an open reading frame encoding an immunoadjuvant selected from SEQ ID_No 12 or a sequence with at least 80% sequence identity or homology.

In one embodiment the vaccine further comprises an mRNA polynucleotide comprising an open reading frame encoding an immunoadjuvant selected from SEQ ID_No 12 or a sequence with at least 90% sequence identity or homology.

In one embodiment the vaccine further comprises an mRNA polynucleotide comprising an open reading frame encoding an immunoadjuvant selected from SEQ ID_No 12 or a sequence with at least 95% sequence identity or homology.

In one embodiment the vaccine further comprises an mRNA polynucleotide comprising an open reading frame encoding an immunoadjuvant selected from SEQ ID_No 12 or a sequence with at least 98% sequence identity or homology.

In one embodiment the vaccine further comprises an mRNA polynucleotide comprising an open reading frame encoding an immunoadjuvant selected from SEQ ID_No 12.

In one embodiment the vaccine further comprises an mRNA polynucleotide comprising an open reading frame encoding an immunoadjuvant selected from SEQ ID_No 13 or a sequence with at least 80% sequence identity or homology.

In one embodiment the vaccine further comprises an mRNA polynucleotide comprising an open reading frame encoding an immunoadjuvant selected from SEQ ID_No 13 or a sequence with at least 90% sequence identity or homology.

In one embodiment the vaccine further comprises an mRNA polynucleotide comprising an open reading frame encoding an immunoadjuvant selected from SEQ ID_No 13 or a sequence with at least 95% sequence identity or homology.

In one embodiment the vaccine further comprises an mRNA polynucleotide comprising an open reading frame encoding an immunoadjuvant selected from SEQ ID_No 13 or a sequence with at least 98% sequence identity or homology.

In one embodiment the vaccine further comprises an mRNA polynucleotide comprising an open reading frame encoding an immunoadjuvant selected from SEQ ID_No 13.

mRNA Modifications

In one embodiment the mRNA polynucleotides described herein comprise one or more modifications relative to the naturally occurring “wild-type” mRNA. Modification refers to modifications of the adenosine (A), guanosine (G), uridine (U), or cytidine (C) nucleotides in at least one of their position, pattern, percent and/or structure. Modifications may comprise naturally-occurring, non-naturally-occurring or a combination of naturally-occurring and non-naturally-occurring modifications. Modifications may be of a sugar, a nucleobase, or an internucleotide linkage (e.g., to a phosphate group). Modifications may be introduced during synthesis or post-synthesis, and may be introduced with chemical synthesis or with a polymerase enzyme. Any of the regions of the mRNA polynucleotide may be modified.

In one embodiment the vaccine comprises an mRNA polynucleotide comprising one or more modifications resulting in reduced degradation of the mRNA after administration relative to an unmodified (i.e. wild-type) polynucleotide.

In one embodiment the vaccine comprises an mRNA polynucleotide comprising one or more modifications resulting in reduced immunogenicity (e.g., a reduced innate response) after administration relative to an unmodified polynucleotide. Modifications of the mRNA polynucleotides described herein may include codon optimisation, capping, for example a five-prime (5′) cap, and uridine modification.

### i) Codon Optimisation

In one embodiment the mRNA polynucleotide may be codon optimized. Codon optimisation ensures the most efficient translation in humans. Codon optimization, in one embodiment, may be used to match codon frequencies in target and host organisms to ensure proper folding; bias G/C content to increase mRNA stability or reduce secondary structures; minimize tandem repeat codons or base runs that may impair gene construction or expression; customize transcriptional and translational control regions; insert or remove protein trafficking sequences; remove/add post translation modification sites in encoded protein (e.g. glycosylation sites); add, remove or shuffle protein domains; insert or delete restriction sites; modify ribosome binding sites and mRNA degradation sites; adjust translational rates to allow the various domains of the protein to fold properly; or to reduce or eliminate problem secondary structures within the polynucleotide. Codon optimization tools, algorithms and services are known in the art—non-limiting examples include services from GeneArt (Life Technologies), DNA2.0 (Menlo Park Calif.) and/or proprietary methods. In one embodiment, the open reading frame (ORF) sequence is optimized using optimization algorithms.

In one embodiment codon-optimized mRNA may, for instance, be one in which the levels of guanosine (G) and/or cytidine (C) residues are enhanced. The G/C-content of nucleic acid molecules may influence the stability of the mRNA. mRNA having an increased amount of guanosine (G) and/or cytidine (C) residues may be functionally more stable than nucleic acids containing a large amount of adenosine (A) and uridine (U) nucleotides. Due to the degeneracy of the genetic code, the modifications work by substituting existing codons for those that promote greater RNA stability without changing the resulting amino acid. The approach is limited to the open reading frame(s) of the mRNA.

In one embodiment codon-optimized mRNA may include uridine depletion relative to an unmodified polynucleotide. Uridine depletion reduces the likelihood of an innate immune response to the mRNA.

### ii) Capping

In one embodiment, the mRNA polynucleotide comprises a 5′ cap. The 5′ cap is a specially altered nucleotide on the 5′ end of the mRNA. mRNA capping may assist with the production of the most biologically active enhancing translation to protein, and least immunogenic mRNA (as in less likely to provoke an innate immune response).

In one embodiment, the mRNA polynucleotide comprises a 5′ cap selected from CleanCap® available from TriLink Biotechnologies® (e.g. those described in WO 2017/053297). Suitable caps include:
CleanCap® Reagent AG for co-transcriptional capping of mRNA: m7G(5′)ppp(5′)(2′OMeA)pG, or
ARCA (Anti Reverse Cap Analog): 3′-O-Me-m⁷G(5′)ppp(5′)G.

5′ capping prevents degradation of the mRNA in the cytoplasm and promotes ribosome recruitment and protein translation.

In one embodiment the vaccine comprises an mRNA polynucleotide comprising an open reading frame encoding a coronavirus antigen or an immunogenic fragment thereof wherein the mRNA polynucleotide comprises a 5′ cap.

In one embodiment the vaccine comprises an mRNA polynucleotide comprising an open reading frame encoding a coronavirus antigen or an immunogenic fragment thereof wherein the mRNA polynucleotide comprises a 5′ cap selected from m7G(5′)ppp(5′)(2′OMeA)pG.

In one embodiment the vaccine comprises an mRNA polynucleotide comprising an open reading frame encoding a coronavirus antigen or an immunogenic fragment thereof wherein the mRNA polynucleotide comprises a 5′ cap selected from 3′-O-Me-m⁷G(5′)ppp(5′)G.

iii) Uridine Modification

In one embodiment, the vaccine comprises an mRNA polynucleotide comprising an open reading frame encoding a coronavirus antigen or an immunogenic fragment thereof, wherein the mRNA polynucleotide comprises modified uridine nucleotides. Uridine modification reduces the likelihood of an innate immune response to the mRNA.

In one embodiment, the vaccine comprises an mRNA polynucleotide comprising an open reading frame encoding a coronavirus antigen or an immunogenic fragment thereof, wherein the mRNA polynucleotide comprises one or more modified uridine nucleotides selected from pseudouridine (ψ), N1-methylpseudouridine (m1ψ), N1-ethylpseudouridine, 2-thiouridine, 4′-thiouridine, 5-methyluridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine and 2′-O-methyl uridine nucleotides.

In one embodiment, the vaccine comprises an mRNA polynucleotide comprising an open reading frame encoding a coronavirus antigen or an immunogenic fragment thereof, wherein the mRNA polynucleotide comprises one or more modified uridine nucleotides selected from N1-methylpseudouridine nucleotide.

In one embodiment, the vaccine comprises an mRNA polynucleotide comprising an open reading frame encoding a coronavirus antigen or an immunogenic fragment thereof, wherein the mRNA polynucleotide comprises modified uridine nucleotides wherein at least 80% of the uridine nucleotides are N1-methylpseudouridine nucleotides.

In one embodiment, the vaccine comprises an mRNA polynucleotide comprising an open reading frame encoding a coronavirus antigen or an immunogenic fragment thereof, wherein the mRNA polynucleotide comprises modified uridine nucleotides wherein at least 90% of the uridine nucleotides are N1-methylpseudouridine nucleotides.

In one embodiment, the vaccine comprises an mRNA polynucleotide comprising an open reading frame encoding a coronavirus antigen or an immunogenic fragment thereof, wherein the mRNA polynucleotide comprises modified uridine nucleotides wherein at least 95% of the uridine nucleotides are N1-methylpseudouridine nucleotides.

In one embodiment, the vaccine comprises an mRNA polynucleotide comprising an open reading frame encoding a coronavirus antigen or an immunogenic fragment thereof, wherein the mRNA polynucleotide comprises modified uridine nucleotides wherein at least 97% of the uridine nucleotides are N1-methylpseudouridine nucleotides.

In one embodiment, the vaccine comprises an mRNA polynucleotide comprising an open reading frame encoding a coronavirus antigen or an immunogenic fragment thereof, wherein the mRNA polynucleotide comprises modified uridine nucleotides wherein at least 99% of the uridine nucleotides are N1-methylpseudouridine nucleotides.

In one embodiment, the vaccine comprises an mRNA polynucleotide comprising an open reading frame encoding a coronavirus antigen or an immunogenic fragment thereof, wherein the mRNA polynucleotide comprises modified uridine nucleotides wherein all uridine nucleotides are N1-methylpseudouridine nucleotides.

### iv) Cytidine Modification

In one embodiment, the vaccine comprises an mRNA polynucleotide comprising modified cytidine nucleotides. Cytidine modification may be employed to impart desirable characteristics such as increased nuclease stability, increased translation or reduced interaction of innate immune receptors with in vitro transcribed RNA.

In one embodiment, the vaccine comprises an mRNA polynucleotide comprising one or more modified cytidine nucleotides selected from N4-acetyl-cytidine (ac4C), 5-methyl-cytidine (m5C), 5-halo-cytidine (e.g., 5-iodo-cytidine), 5-hydroxymethyl-cytidine (hm5C), 1-methyl-pseudoisocytidine, 2-thio-cytidine (s2C), 5-methylcytidine-5′-triphosphate (5-methyl-CTP), and 2-thio-5-methyl-cytidine.

In one embodiment the mRNA polynucleotide comprises no cytidine modifications.

In one embodiment, the vaccine comprises an mRNA polynucleotide comprising an open reading frame encoding a coronavirus antigen or an immunogenic fragment thereof, wherein the mRNA polynucleotide comprises modified uridine nucleotides wherein at least 80% of the uridine nucleotides are N1-methylpseudouridine nucleotides and the cytidine nucleotides are unmodified.

In one embodiment, the vaccine comprises an mRNA polynucleotide comprising an open reading frame encoding a coronavirus antigen or an immunogenic fragment thereof, wherein the mRNA polynucleotide comprises modified uridine nucleotides wherein at least 90% of the uridine nucleotides are N1-methylpseudouridine nucleotides and the cytidine nucleotides are unmodified.

In one embodiment, the vaccine comprises an mRNA polynucleotide comprising an open reading frame encoding a coronavirus antigen or an immunogenic fragment thereof, wherein the mRNA polynucleotide comprises modified uridine nucleotides wherein at least 95% of the uridine nucleotides are N1-methylpseudouridine nucleotides and the cytidine nucleotides are unmodified.

In one embodiment, the vaccine comprises an mRNA polynucleotide comprising an open reading frame encoding a coronavirus antigen or an immunogenic fragment thereof, wherein the mRNA polynucleotide comprises modified uridine nucleotides wherein at least 97% of the uridine nucleotides are N1-methylpseudouridine nucleotides and the cytidine nucleotides are unmodified.

In one embodiment, the vaccine comprises an mRNA polynucleotide comprising an open reading frame encoding a coronavirus antigen or an immunogenic fragment thereof, wherein the mRNA polynucleotide comprises modified uridine nucleotides wherein at least 99% of the uridine nucleotides are N1-methylpseudouridine nucleotides and the cytidine nucleotides are unmodified.

In one embodiment, the vaccine comprises an mRNA polynucleotide comprising an open reading frame encoding a coronavirus antigen or an immunogenic fragment thereof, wherein the mRNA polynucleotide comprises modified uridine nucleotides wherein all uridine nucleotides are N1-methylpseudouridine nucleotides and the cytidine nucleotides are unmodified.

In one embodiment the vaccine comprises an mRNA polynucleotide comprising one or more modifications selected from a 5′ cap and uridine modification.

In one embodiment the vaccine comprises an mRNA polynucleotide comprising two modifications selected from a 5′ cap and uridine modification.

In one embodiment the vaccine comprises an mRNA polynucleotide uniformly modified (e.g., fully modified throughout the entire sequence) for a particular modification.

In one embodiment the mRNA polynucleotides described herein comprise one or more modifications selected from a 5′ cap, and uridine modification fully modified throughout the entire sequence.

In one embodiment the vaccine comprises an mRNA polynucleotide comprising one or more modifications selected from a 5′ cap, and uridine modification in one region of the polynucleotide.

### Amphipathic Cell Penetrating Peptide

In accordance with convention, the peptides described herein are drawn “N-terminus” first, i.e. on the left hand side.

In one embodiment the vaccine comprises an amphipathic cell penetrating peptide comprising or consisting of the amino acid sequence WEARLARALARALARHLARALARALRACEA (SEQ ID_No 1) or a sequence with at least 80% sequence identity or homology.

In one embodiment the vaccine comprises an amphipathic cell penetrating peptide comprising the amino acid sequence WEARLARALARALARHLARALARALRACEA (SEQ ID_No 1) or a sequence with at least 80% sequence identity or homology.

In one embodiment the vaccine comprises an amphipathic cell penetrating peptide consisting of the amino acid sequence WEARLARALARALARHLARALARALRACEA (SEQ ID_No 1) or a sequence with at least 80% sequence identity or homology.

In one embodiment the vaccine comprises an amphipathic cell penetrating peptide comprising or consisting of the amino acid sequence WEARLARALARALARHLARALARALRACEA (SEQ ID_No 1) or a sequence with at least 85% sequence identity or homology.

In one embodiment the vaccine comprises an amphipathic cell penetrating peptide comprising the amino acid sequence WEARLARALARALARHLARALARALRACEA (SEQ ID_No 1) or a sequence with at least 85% sequence identity or homology.

In one embodiment the vaccine comprises an amphipathic cell penetrating peptide consisting of the amino acid sequence WEARLARALARALARHLARALARALRACEA (SEQ ID_No 1) or a sequence with at least 85% sequence identity or homology.

In one embodiment the vaccine comprises an amphipathic cell penetrating peptide comprising or consisting of the amino acid sequence WEARLARALARALARHLARALARALRACEA (SEQ ID_No 1) or a sequence with at least 90% sequence identity or homology.

In one embodiment the vaccine comprises an amphipathic cell penetrating peptide comprising the amino acid sequence WEARLARALARALARHLARALARALRACEA (SEQ ID_No 1) or a sequence with at least 90% sequence identity or homology.

In one embodiment the vaccine comprises an amphipathic cell penetrating peptide consisting of the amino acid sequence WEARLARALARALARHLARALARALRACEA (SEQ ID_No 1) or a sequence with at least 90% sequence identity or homology.

In one embodiment the vaccine comprises an amphipathic cell penetrating peptide comprising or consisting of the amino acid sequence WEARLARALARALARHLARALARALRACEA (SEQ ID_No 1) or a sequence with at least 95% sequence identity or homology.

In one embodiment the vaccine comprises an amphipathic cell penetrating peptide comprising the amino acid sequence WEARLARALARALARHLARALARALRACEA (SEQ ID_No 1) or a sequence with at least 95% sequence identity or homology.

In one embodiment the vaccine comprises an amphipathic cell penetrating peptide consisting of the amino acid sequence WEARLARALARALARHLARALARALRACEA (SEQ ID_No 1) or a sequence with at least 95% sequence identity or homology.

In one embodiment the vaccine comprises an amphipathic cell penetrating peptide comprising or consisting of the amino acid sequence WEARLARALARALARHLARALARALRACEA (SEQ ID_No 1).

In one embodiment the vaccine comprises an amphipathic cell penetrating peptide comprising the amino acid sequence WEARLARALARALARHLARALARALRACEA (SEQ ID_No 1).

In one embodiment the vaccine comprises an amphipathic cell penetrating peptide consisting of the amino acid sequence WEARLARALARALARHLARALARALRACEA (SEQ ID_No 1).

In one embodiment a sequence with at least 80% sequence identity or homology to WEARLARALARALARHLARALARALRACEA (SEQ ID_No 1) consists of less than or equal to 35 amino acid residues.

In one embodiment a sequence with at least 80% sequence identity or homology to WEARLARALARALARHLARALARALRACEA (SEQ ID_No 1) consists of less than or equal to 30 amino acid residues.

In one embodiment a sequence with at least 80% sequence identity or homology to WEARLARALARALARHLARALARALRACEA (SEQ ID_No 1) consists of 26-30 amino acid residues.

In one embodiment a sequence with at least 80% sequence identity or homology to WEARLARALARALARHLARALARALRACEA (SEQ ID_No 1) comprises at least 5 arginine residues (R).

In one embodiment a sequence with at least 80% sequence identity or homology to WEARLARALARALARHLARALARALRACEA (SEQ ID_No 1) comprises at least 6 arginine residues (R).

In one embodiment a sequence with at least 80% sequence identity or homology to WEARLARALARALARHLARALARALRACEA (SEQ ID_No 1) comprises at least 10 alanine residues (A).

In one embodiment a sequence with at least 80% sequence identity or homology to WEARLARALARALARHLARALARALRACEA (SEQ ID_No 1) comprises at least 12 alanine residues (A).

In one embodiment a sequence with at least 80% sequence identity or homology to WEARLARALARALARHLARALARALRACEA (SEQ ID_No 1) comprises at least 5 leucine residues (L).

In one embodiment a sequence with at least 80% sequence identity or homology to WEARLARALARALARHLARALARALRACEA (SEQ ID_No 1) comprises at least 6 leucine residues (L).

In one embodiment a sequence with at least 80% sequence identity or homology to WEARLARALARALARHLARALARALRACEA (SEQ ID_No 1) comprises at least one cysteine residue (C).

In one embodiment a sequence with at least 80% sequence identity or homology to WEARLARALARALARHLARALARALRACEA (SEQ ID_No 1) comprises at least two but no more than three glutamic acid (E) residues.

In one embodiment a sequence with at least 80% sequence identity or homology to WEARLARALARALARHLARALARALRACEA (SEQ ID_No 1) comprises at least 6 arginine residues (R), at least 12 Alanine Residues (A), at least 6 leucine residues (L), optionally at least one cysteine residue (C) and at least two but no more than three glutamic acids residues (E).

In one embodiment a sequence with at least 80% sequence identity or homology to WEARLARALARALARHLARALARALRACEA (SEQ ID_No 1) comprises the consensus sequences EARLARALARALAR and/or LARALARALRA.

In one embodiment a sequence with at least 80% sequence identity or homology to WEARLARALARALARHLARALARALRACEA (SEQ ID_No 1) comprises the consensus sequence EARLARALARALAR.

In one embodiment a sequence with at least 80% sequence identity or homology to WEARLARALARALARHLARALARALRACEA (SEQ ID_No 1) comprises the consensus sequence LARALARALRA.

In one embodiment a sequence with at least 80% sequence identity or homology to WEARLARALARALARHLARALARALRACEA (SEQ ID_No 1) comprises the consensus sequences EARLARALARALAR and LARALARALRA.

In one embodiment a sequence with at least 80% sequence identity or homology to WEARLARALARALARHLARALARALRACEA (SEQ ID_No 1) does not comprise glycine (G).

A Sequence with at Least 80% Sequence Identity or Homology

Any of a variety of sequence alignment methods can be used to determine percent sequence identity, including, without limitation, global methods, local methods and hybrid methods, such as, e.g., segment approach methods. Protocols to determine percent identity are routine procedures within the scope of one skilled in the art. Global methods align sequences from the beginning to the end of the molecule and determine the best alignment by adding up scores of individual residue pairs and by imposing gap penalties. Conventional methods include Altschul et al., Bull. Math. Bio. 48: 603-16, 1986 and Henikoff and Henikoff, Proc. Natl. Acad. Sci. USA 89:10915-19, 1992 where two amino acid sequences are aligned to optimize the alignment scores using a gap opening penalty of 10, a gap extension penalty of 1, and the “blosum 62” scoring matrix of Henikoff and Henikoff.

“Sequence identity” between two or more sequences is expressed as a percentage and is a function of the number of identical positions shared by the sequences. Thus, sequence identity may be calculated as the number of identical amino acids or nucleotides divided by the total number of amino acids or nucleotides, multiplied by 100. Calculations of % sequence identity may also take into account the number of gaps, and the length of each gap that needs to be introduced to optimize alignment of two or more sequences. Sequence comparisons and the determination of percent identity between two or more sequences can be carried out using specific mathematical algorithms, such as BLAST, which will be familiar to a skilled person.

Homologous sequences may be characterized as having one or more amino acid or nucleotide substitutions, deletions or additions/insertions. These changes are of a minor nature that do not significantly affect the folding or activity of the peptide. These may be small amino acid or nucleotide substitutions; small deletions; and small terminal extensions or other small additions/insertions. An algorithm (e.g. BLAST) looks for the minimal number of edit operations (inserts, deletes, and substitutions) in order to transform the one sequence into an exact copy of the other sequence and assigns a % similarity (homology).

In one embodiment the term “sequence identity or homology” refers to sequence identity.

In one embodiment the term “sequence identity or homology” refers to sequence homology.

### Nanoparticles

In one embodiment the vaccines described herein comprise or consist of nanoparticles. Nanoparticles may be formed by self-assembly by adding the mRNA polynucleotide, and the amphipathic cell penetrating peptide together in ultrapure water. The formulation may be lyophilised and then reconstituted for administration.

In one embodiment there is provided a nanoparticle formulation comprising:
i) an mRNA polynucleotide comprising an open reading frame encoding a coronavirus antigen or an immunogenic fragment thereof; and
ii) an amphipathic cell penetrating peptide comprising or consisting of the amino acid sequence WEARLARALARALARHLARALARALRACEA (SEQ ID_No 1) or a sequence with at least 80% sequence identity or homology.

In one embodiment there is provided a vaccine as described herein comprising a nanoparticle formulation of:
i) an mRNA polynucleotide comprising an open reading frame encoding a coronavirus antigen or an immunogenic fragment thereof; and
ii) an amphipathic cell penetrating peptide comprising or consisting of the amino acid sequence WEARLARALARALARHLARALARALRACEA (SEQ ID_No 1) or a sequence with at least 80% sequence identity or homology.

In one embodiment there is provided a vaccine as described herein formulated in a nanoparticle.

In one embodiment there is provided a nanoparticle comprising a vaccine as described herein.

In one embodiment there is provided a nanoparticle formulation comprising a vaccine as described herein.

In one embodiment there is provided a vaccine as described herein, comprising nanoparticles.

In one embodiment there is provided a vaccine as described herein, comprising nanoparticles with a Z-Average of 30-150 nm. The Z average is the intensity weighted mean hydrodynamic size of the ensemble collection of particles measured by dynamic light scattering (DLS).

In one embodiment there is provided a vaccine as described herein, comprising nanoparticles with a Z-Average of 60-100 nm.

In one embodiment there is provided a vaccine as described herein, comprising nanoparticles with a polydispersity index of =<0.3. The polydispersity index (PI) is a measure of the heterogeneity of a sample based on size.

In one embodiment there is provided a vaccine as described herein, comprising nanoparticles with a polydispersity index of =<0.5.

In one embodiment there is provided a vaccine as described herein, comprising nanoparticles with a net positive charge at a neutral pH.

N:P Ratio of mRNA:Amphipathic Cell Penetrating Peptide

The amount of the RALA peptide to mRNA is indicated by the N:P ratio, and represents the molar ratio of positively charged nitrogen atoms in the peptide to negatively charged phosphates in the mRNA. The mRNA quoted in the ratio may indicate one mRNA, or a mixture of mRNAs depending on the product.

N:P ratio is widely used to describe the contents of peptide or protein based nucleic acid nanoparticles due to its simplicity in comparison to masses. It is the molar ratio of positively charged nitrogen atoms in the amino acids to the negatively charged phosphates contributed by the nucleic acid backbone and can be described as the mass of protein or peptide required to neutralise the charge of 1 μg of nucleic acid. The N:P ratio can be calculated using the following equation:

$$N : P = \frac{MRALA}{MmRNA \cdot CNP}$$

where M_{RALA} is the mass of protein in the nanoparticle, M_{mRNA} is the mass of mRNA in the nanoparticle and C_{NP} is the N:P constant. The N:P constant is the ratio of the positive charge density of the amino acid chain to the negative charge density of the mRNA backbone with charge density being defined as the charge divided by the molecular mass. This N:P constant can be calculated based on the knowledge that arginine is the positively charged amino acids and that the mass and charge of the bases in the mRNA backbone are constant leaving only molecular mass of the protein and charge of the protein as variables. As such the N:P constant calculation can be simplified to:

$$NP 1 = 1 \times \frac{MWRALA}{340 * QRALA} = \frac{3327.98}{340 * 7} = 1.4$$

Where Q_{RALA} is the positive charge provided by the seven arginine residues in the sequence of RALA, and 340 is the average molecular weight of an mRNA nucleoside.

For example an N:P ratio (peptide to mRNA ratio) of 1:1 is 1.40 μg peptide (SEQ ID_No 1):1 μg mRNA. An N:P ratio of 9:1 is 12.6 μg peptide (SEQ ID_No 1):1 μg mRNA.

In one embodiment, the N:P ratio of the amphipathic cell penetrating peptide:mRNA polynucleotide in the vaccine may be varied. This may have a beneficial effect on the physiochemical characteristics (for example the Z-Average, zeta potential (particle charge), and/or polydispersity index), the cellular uptake, and/or the treatment efficacy.

In one embodiment the N:P ratio of amphipathic cell penetrating peptide:mRNA polynucleotide in the vaccine is about 1-12:1.

In one embodiment the N:P ratio of amphipathic cell penetrating peptide:mRNA polynucleotide is 1-12:1.

In one embodiment the N:P ratio of amphipathic cell penetrating peptide:mRNA polynucleotide in the vaccine is about 5-12:1.

In one embodiment the N:P ratio of amphipathic cell penetrating peptide:mRNA polynucleotide in the vaccine is 5-12:1.

In one embodiment the N:P ratio of amphipathic cell penetrating peptide:mRNA polynucleotide in the vaccine is about 7-10:1.

In one embodiment the N:P ratio of amphipathic cell penetrating peptide:mRNA polynucleotide in the vaccine is 7-10:1.

In one embodiment the N:P ratio of amphipathic cell penetrating peptide:mRNA polynucleotide in the vaccine is about 8.5-9.5:1.

In one embodiment the N:P ratio of amphipathic cell penetrating peptide:mRNA polynucleotide in the vaccine is 8.5-9.5:1.

In one embodiment the N:P ratio of amphipathic cell penetrating peptide:mRNA polynucleotide in the vaccine is 1:1.

In one embodiment the N:P ratio of amphipathic cell penetrating peptide:mRNA polynucleotide in the vaccine is about 1:1.

In one embodiment the N:P ratio of amphipathic cell penetrating peptide:mRNA polynucleotide in the vaccine is 2:1.

In one embodiment the N:P ratio of amphipathic cell penetrating peptide:mRNA polynucleotide in the vaccine is about 2:1.

In one embodiment the N:P ratio of amphipathic cell penetrating peptide:mRNA polynucleotide in the vaccine is 3:1.

In one embodiment the N:P ratio of amphipathic cell penetrating peptide:mRNA polynucleotide in the vaccine is about 3:1.

In one embodiment the N:P ratio of amphipathic cell penetrating peptide:mRNA polynucleotide in the vaccine is 4:1.

In one embodiment the N:P ratio of amphipathic cell penetrating peptide:mRNA polynucleotide in the vaccine is about 4:1.

In one embodiment the N:P ratio of amphipathic cell penetrating peptide:mRNA polynucleotide in the vaccine is 5:1.

In one embodiment the N:P ratio of amphipathic cell penetrating peptide:mRNA polynucleotide in the vaccine is about 5:1.

In one embodiment the N:P ratio of amphipathic cell penetrating peptide:mRNA polynucleotide in the vaccine is 6:1.

In one embodiment the N:P ratio of amphipathic cell penetrating peptide:mRNA polynucleotide in the vaccine is about 6:1.

In one embodiment the N:P ratio of amphipathic cell penetrating peptide:mRNA polynucleotide in the vaccine is 7:1.

In one embodiment the N:P ratio of amphipathic cell penetrating peptide:mRNA polynucleotide in the vaccine is about 7:1.

In one embodiment the N:P ratio of amphipathic cell penetrating peptide:mRNA polynucleotide in the vaccine is 8:1.

In one embodiment the N:P ratio of amphipathic cell penetrating peptide:mRNA polynucleotide in the vaccine is about 8:1.

In one embodiment the N:P ratio of amphipathic cell penetrating peptide:mRNA polynucleotide in the vaccine is 9:1.

In one embodiment the N:P ratio of amphipathic cell penetrating peptide:mRNA polynucleotide in the vaccine is about 9:1.

In one embodiment the N:P ratio of amphipathic cell penetrating peptide:mRNA polynucleotide in the vaccine is 10:1.

In one embodiment the N:P ratio of amphipathic cell penetrating peptide:mRNA polynucleotide in the vaccine is about 10:1.

In one embodiment the N:P ratio of amphipathic cell penetrating peptide:mRNA polynucleotide in the vaccine is 11:1.

In one embodiment the N:P ratio of amphipathic cell penetrating peptide:mRNA polynucleotide in the vaccine is about 11:1.

In one embodiment the N:P ratio of amphipathic cell penetrating peptide:mRNA polynucleotide in the vaccine is 12:1.

In one embodiment the N:P ratio of amphipathic cell penetrating peptide:mRNA polynucleotide in the vaccine is about 12:1.

### Bulking Agents, Cryoprotectants and Agents Inferring Tonicity

In one embodiment, a bulking agent may be added prior to lyophilisation of nanoparticles for transport and storage. Bulking agents are additives that increase the bulk-volume of a product without affecting its properties.

In one embodiment, a cryoprotectant may be added prior to lyophilisation of nanoparticles. A cryoprotectant is a substance used to protect biological material from freezing damage.

In one embodiment, a solute may be added to infer tonicity, e.g. to produce an isotonic formulation once water is added to the formulation. An isotonic formulation possesses the same concentration of solutes as the blood, i.e. 290-310 mOsmol/kg.

In one embodiment, the osmolality of a solution of a vaccine as described herein in water is 10-1000 mOsmol/kg.

In one embodiment, the osmolality of a solution of a vaccine as described herein in water is 100-500 mOsmol/kg.

In one embodiment, the osmolality of a solution of a vaccine as described herein in water is 200-400 mOsmol/kg.

In one embodiment, the osmolality of a solution of a vaccine as described herein in water is 290-310 mOsmol/kg.

In one embodiment, the osmolality of a solution of a vaccine as described herein in water is 280-300 mOsmol/kg.

In one embodiment, the osmolality of a solution of a vaccine as described herein in water is about 300 mOsmol/kg.

In one embodiment, the osmolality of a solution of a vaccine as described herein in water is 300 mOsmol/kg.

In one embodiment, the osmolality of a solution of a vaccine as described herein in water is about 295 mOsmol/kg.

In one embodiment, the osmolality of a solution of a vaccine as described herein in water is 295 mOsmol/kg.

Suitable bulking agents include trehalose, sucrose, mannose, dextrose or any mixture of such agents. These agents may also be employed as cryoprotectants and/or agents to infer tonicity.

In one embodiment, the vaccines described herein additionally comprise trehalose, sucrose, mannose, dextrose or any mixture of such agents.

In one embodiment, the vaccines described herein additionally comprise >85% w/w trehalose, sucrose, mannose, dextrose or any mixture of such agents.

In one embodiment, the vaccines described herein additionally comprise >90% w/w trehalose, sucrose, mannose, dextrose or any mixture of such agents.

In one embodiment, the vaccines described herein additionally comprise >95% w/w trehalose, sucrose, mannose, dextrose or any mixture of such agents.

In one embodiment the vaccine described herein comprises a bulking agent.

In one embodiment the vaccine described herein comprises a bulking agent selected from trehalose, sucrose, mannose and dextrose.

In one embodiment the vaccine described herein comprises trehalose.

In one embodiment the vaccine described herein comprises trehalose.

In one embodiment the vaccines described herein additionally comprise trehalose.

In one embodiment the vaccines described herein additionally comprise >85% w/w trehalose.

In one embodiment the vaccines described herein additionally comprise >90% w/w trehalose.

In one embodiment the vaccines described herein additionally comprise >95% w/w trehalose.

In one embodiment the vaccines described herein additionally comprise >98% w/w trehalose.

In one embodiment the vaccines described herein additionally comprise about 99% w/w trehalose.

In one embodiment the vaccines described herein additionally comprise 99% w/w trehalose.

In one embodiment the vaccine described herein comprises sucrose.

In one embodiment the vaccines described herein additionally comprise sucrose.

In one embodiment the vaccines described herein additionally comprise >85% w/w sucrose.

In one embodiment the vaccines described herein additionally comprise >90% w/w sucrose.

In one embodiment the vaccines described herein additionally comprise >95% w/w sucrose.

In one embodiment the vaccine described herein comprises mannose.

In one embodiment the vaccines described herein additionally comprise mannose.

In one embodiment the vaccines described herein additionally comprise >85% w/w mannose.

In one embodiment the vaccines described herein additionally comprise >90% w/w mannose.

In one embodiment the vaccines described herein additionally comprise >95% w/w mannose.

In one embodiment the vaccine described herein comprises dextrose.

In one embodiment the vaccines described herein additionally comprise dextrose.

In one embodiment the vaccines described herein additionally comprise >85% w/w dextrose.

In one embodiment the vaccines described herein additionally comprise >90% w/w dextrose.

In one embodiment the vaccines described herein additionally comprise >95% w/w dextrose.

In one embodiment the vaccine as described herein comprises:
(i) 0.01-1.5% w/w one or more mRNA polynucleotides comprising open reading frames encoding a coronavirus antigen or an immunogenic fragment thereof;
(ii) 1-15% w/w an amphipathic cell penetrating peptide comprising or consisting of the amino acid sequence WEARLARALARALARHLARALARALRACEA (SEQ ID_No 1) or a sequence with at least 80% sequence identity or homology; and
(iii) 80-99% w/w trehalose.

In one embodiment the vaccine as described herein comprises:
(i) 0.01-1.5% w/w one or more mRNA polynucleotides comprising open reading frames encoding a coronavirus S, M and/or E protein or an immunogenic fragment thereof;
(ii) 1-15% w/w an amphipathic cell penetrating peptide comprising or consisting of the amino acid sequence WEARLARALARALARHLARALARALRACEA (SEQ ID_No 1) or a sequence with at least 80% sequence identity or homology; and
(iii) 80-99% w/w trehalose.

In one embodiment the vaccine as described herein comprises:
(i) 0.01-1.5% w/w 3 mRNA polynucleotides comprising open reading frames one encoding a coronavirus S protein or an immunogenic fragment thereof, one encoding a coronavirus M protein or an immunogenic fragment thereof and one encoding a coronavirus E protein or an immunogenic fragment thereof;
(ii) 1-15% w/w an amphipathic cell penetrating peptide comprising or consisting of the amino acid sequence WEARLARALARALARHLARALARALRACEA (SEQ ID_No 1) or a sequence with at least 80% sequence identity or homology; and
(iii) 80-99% w/w trehalose.

In one embodiment the vaccine as described herein comprises:
(i) 0.01-0.2% w/w an mRNA polynucleotides comprising open reading frames encoding a coronavirus S protein or an immunogenic fragment thereof;
(ii) 0.01-0.04% w/w an mRNA polynucleotides comprising open reading frames encoding a coronavirus M protein or an immunogenic fragment thereof;
(iii) 0.001-0.02% w/w an mRNA polynucleotides comprising open reading frames encoding a coronavirus E protein or an immunogenic fragment thereof;
(iv) 1.5-2.5% w/w an amphipathic cell penetrating peptide comprising or consisting of the amino acid sequence WEARLARALARALARHLARALARALRACEA (SEQ ID_No 1) or a sequence with at least 80% sequence identity or homology; and
(v) 80-99% w/w trehalose.

In one embodiment the vaccine as described herein comprises:
(i) 0.01-0.2% w/w an mRNA polynucleotide comprising an open reading frame comprising a sequence selected from SEQ ID_No 2 or SEQ ID_No 5;
(ii) 0.01-0.04% w/w an mRNA polynucleotide comprising an open reading frame comprising a sequence selected from SEQ ID_No 3;
(iii) 0.001-0.02% w/w an mRNA polynucleotide comprising an open reading frame comprising a sequence selected from SEQ ID_No 2 or SEQ ID_No 4;
(iv) 1.5-2.5% w/w an amphipathic cell penetrating peptide comprising or consisting of the amino acid sequence WEARLARALARALARHLARALARALRACEA (SEQ ID_No 1) or a sequence with at least 80% sequence identity or homology; and
(v) 80-99% w/w trehalose.

In one embodiment the vaccine as described herein comprises:
(i) about 0.12% w/w an mRNA polynucleotides comprising open reading frames encoding a coronavirus S protein or an immunogenic fragment thereof;
(ii) about 0.03% w/w an mRNA polynucleotides comprising open reading frames encoding a coronavirus M protein or an immunogenic fragment thereof;
(iii) about 0.01% w/wan mRNA polynucleotides comprising open reading frames encoding a coronavirus E protein or an immunogenic fragment thereof;
(iv) about 2.09% w/w an amphipathic cell penetrating peptide comprising or consisting of the amino acid sequence WEARLARALARALARHLARALARALRACEA (SEQ ID_No 1) or a sequence with at least 80% sequence identity or homology; and
(v) about 97.75% w/w trehalose.

In one embodiment the vaccine as described herein comprises:
(i) 0.12% w/w an mRNA polynucleotide comprising an open reading frame comprising a sequence selected from SEQ ID_No 2 or SEQ ID_No 5;
(ii) 0.03% w/w an mRNA polynucleotide comprising an open reading frame comprising a sequence selected from SEQ ID_No 3;
(iii) 0.01% w/w an mRNA polynucleotides comprising open reading frames encoding a coronavirus E protein or an immunogenic fragment thereof;
(iv) 2.09% w/w an amphipathic cell penetrating peptide comprising or consisting of the amino acid sequence WEARLARALARALARHLARALARALRACEA (SEQ ID_No 1) or a sequence with at least 80% sequence identity or homology; and
(v) 97.75% w/w trehalose.

In one embodiment the vaccine as described herein comprises:
(i) 0.2-0.3% w/w an mRNA polynucleotides comprising open reading frames encoding a coronavirus S protein or an immunogenic fragment thereof;
(ii) 0.03-0.07% w/w an mRNA polynucleotides comprising open reading frames encoding a coronavirus M protein or an immunogenic fragment thereof;
(iii) 0.01-0.05% w/w an mRNA polynucleotides comprising open reading frames encoding a coronavirus E protein or an immunogenic fragment thereof;
(iv) 3.5-3.5% w/w an amphipathic cell penetrating peptide comprising or consisting of the amino acid sequence WEARLARALARALARHLARALARALRACEA (SEQ ID_No 1) or a sequence with at least 80% sequence identity or homology; and
(v) 94-97% w/w trehalose.

In one embodiment the vaccine as described herein comprises:
(i) 0.2-0.3% w/w an mRNA polynucleotide comprising an open reading frame comprising a sequence selected from SEQ ID_No 2 or SEQ ID_No 5;
(ii) 0.03-0.07% w/w an mRNA polynucleotide comprising an open reading frame comprising a sequence selected from SEQ ID_No 3;
(iii) 0.01-0.05% w/w an mRNA polynucleotide comprising an open reading frame comprising a sequence selected from SEQ ID_No 2 or SEQ ID_No 4;
(iv) 3.5-3.5% w/w an amphipathic cell penetrating peptide comprising or consisting of the amino acid sequence WEARLARALARALARHLARALARALRACEA (SEQ ID_No 1) or a sequence with at least 80% sequence identity or homology; and
(v) 94-97% w/w trehalose.

In one embodiment the vaccine as described herein comprises:
(i) about 0.24% w/w an mRNA polynucleotides comprising open reading frames encoding a coronavirus S protein or an immunogenic fragment thereof;
(ii) about 0.05% w/w an mRNA polynucleotides comprising open reading frames encoding a coronavirus M protein or an immunogenic fragment thereof;
(iii) about 0.03% w/wan mRNA polynucleotides comprising open reading frames encoding a coronavirus E protein or an immunogenic fragment thereof;
(iv) about 4.02% w/w an amphipathic cell penetrating peptide comprising or consisting of the amino acid sequence WEARLARALARALARHLARALARALRACEA (SEQ ID_No 1) or a sequence with at least 80% sequence identity or homology; and
(v) about 95.66% w/w trehalose.

In one embodiment the vaccine as described herein comprises:
(i) 0.24% w/w an mRNA polynucleotide comprising an open reading frame comprising a sequence selected from SEQ ID_No 2 or SEQ ID_No 5;
(ii) 0.05% w/w an mRNA polynucleotide comprising an open reading frame comprising a sequence selected from SEQ ID_No 3;
(iii) 0.03% w/w an mRNA polynucleotides comprising open reading frames encoding a coronavirus E protein or an immunogenic fragment thereof;
(iv) 4.02% w/w an amphipathic cell penetrating peptide comprising or consisting of the amino acid sequence WEARLARALARALARHLARALARALRACEA (SEQ ID_No 1) or a sequence with at least 80% sequence identity or homology; and
(v) 95.66% w/w trehalose.

In one embodiment the vaccine as described herein comprises:
(i) 0.75-0.95% w/w an mRNA polynucleotides comprising open reading frames encoding a coronavirus S protein or an immunogenic fragment thereof;
(ii) 0.1-0.3% w/w an mRNA polynucleotides comprising open reading frames encoding a coronavirus M protein or an immunogenic fragment thereof;
(iii) 0.01-0.2% w/w an mRNA polynucleotides comprising open reading frames encoding a coronavirus E protein or an immunogenic fragment thereof;
(iv) 13-15% w/w an amphipathic cell penetrating peptide comprising or consisting of the amino acid sequence WEARLARALARALARHLARALARALRACEA (SEQ ID_No 1) or a sequence with at least 80% sequence identity or homology; and
(v) 83-86% w/w trehalose.

In one embodiment the vaccine as described herein comprises:
(i) 0.75-0.95% w/w an mRNA polynucleotide comprising an open reading frame comprising a sequence selected from SEQ ID_No 2 or SEQ ID_No 5;
(ii) 0.1-0.3% w/w an mRNA polynucleotide comprising an open reading frame comprising a sequence selected from SEQ ID_No 3;
(iii) 0.01-0.2% w/w an mRNA polynucleotide comprising an open reading frame comprising a sequence selected from SEQ ID_No 2 or SEQ ID_No 4;
(iv) 13-15% w/w an amphipathic cell penetrating peptide comprising or consisting of the amino acid sequence WEARLARALARALARHLARALARALRACEA (SEQ ID_No 1) or a sequence with at least 80% sequence identity or homology; and
(v) 83-86% w/w trehalose.

In one embodiment the vaccine as described herein comprises:
(i) about 0.84% w/w an mRNA polynucleotides comprising open reading frames encoding a coronavirus S protein or an immunogenic fragment thereof;
(ii) about 0.19% w/w an mRNA polynucleotides comprising open reading frames encoding a coronavirus M protein or an immunogenic fragment thereof;
(iii) about 0.1% w/wan mRNA polynucleotides comprising open reading frames encoding a coronavirus E protein or an immunogenic fragment thereof;
(iv) about 14.16% w/w an amphipathic cell penetrating peptide comprising or consisting of the amino acid sequence WEARLARALARALARHLARALARALRACEA (SEQ ID_No 1) or a sequence with at least 80% sequence identity or homology; and
(v) about 84.7% w/w trehalose.

In one embodiment the vaccine as described herein comprises:
(i) 0.84% w/w an mRNA polynucleotide comprising an open reading frame comprising a sequence selected from SEQ ID_No 2 or SEQ ID_No 5;
(ii) 0.19% w/w an mRNA polynucleotide comprising an open reading frame comprising a sequence selected from SEQ ID_No 3;
(iii) 0.1% w/w an mRNA polynucleotides comprising open reading frames encoding a coronavirus E protein or an immunogenic fragment thereof;
(iv) 14.16% w/w an amphipathic cell penetrating peptide comprising or consisting of the amino acid sequence WEARLARALARALARHLARALARALRACEA (SEQ ID_No 1) or a sequence with at least 80% sequence identity or homology; and
(v) 84.7% w/w trehalose.

### Formulations in Water

The vaccines described herein may be conveniently formulated in water, particularly ultrapure water, for ease of administration, particularly via intravenous, intramuscular or intradermal injection.

In one embodiment the vaccine as described herein comprises:
(i) 0.01-1.5% w/w one or more mRNA polynucleotides comprising open reading frames encoding a coronavirus antigen or an immunogenic fragment thereof;
(ii) 1-15% w/w an amphipathic cell penetrating peptide comprising or consisting of the amino acid sequence WEARLARALARALARHLARALARALRACEA (SEQ ID_No 1) or a sequence with at least 80% sequence identity or homology; and
(iii) 80-99% w/w trehalose;
   formulated as an isotonic formulation in water with an osmolality of 290-310 mOsmol/kg.

In one embodiment the vaccine as described herein comprises:
(i) 0.01-1.5% w/w one or more mRNA polynucleotides comprising open reading frames encoding a coronavirus S, M and/or E protein or an immunogenic fragment thereof;
(ii) 1-15% w/w an amphipathic cell penetrating peptide comprising or consisting of the amino acid sequence WEARLARALARALARHLARALARALRACEA (SEQ ID_No 1) or a sequence with at least 80% sequence identity or homology; and
(iii) 80-99% w/w trehalose;
formulated as an isotonic formulation in water with an osmolality of 290-310 mOsmol/kg.

In one embodiment the vaccine as described herein comprises:
(i) 0.01-1.5% w/w 3 mRNA polynucleotides comprising open reading frames one encoding a coronavirus S protein or an immunogenic fragment thereof, one encoding a coronavirus M protein or an immunogenic fragment thereof and one encoding a coronavirus E protein or an immunogenic fragment thereof;
(ii) 1-15% w/w an amphipathic cell penetrating peptide comprising or consisting of the amino acid sequence WEARLARALARALARHLARALARALRACEA (SEQ ID_No 1) or a sequence with at least 80% sequence identity or homology; and
(iii) 80-99% w/w trehalose;
formulated as an isotonic formulation in water with an osmolality of 290-310 mOsmol/kg.

In one embodiment the vaccine as described herein comprises:
(i) 0.01-0.2% w/w an mRNA polynucleotides comprising open reading frames encoding a coronavirus S protein or an immunogenic fragment thereof;
(ii) 0.01-0.04% w/w an mRNA polynucleotides comprising open reading frames encoding a coronavirus M protein or an immunogenic fragment thereof;
(iii) 0.001-0.02% w/w an mRNA polynucleotides comprising open reading frames encoding a coronavirus E protein or an immunogenic fragment thereof;
(iv) 1.5-2.5% w/w an amphipathic cell penetrating peptide comprising or consisting of the amino acid sequence WEARLARALARALARHLARALARALRACEA (SEQ ID_No 1) or a sequence with at least 80% sequence identity or homology; and
(v) 80-99% w/w trehalose;
formulated as an isotonic formulation in water with an osmolality of 290-310 mOsmol/kg.

In one embodiment the vaccine as described herein comprises:
(i) 0.01-0.2% w/w an mRNA polynucleotide comprising an open reading frame comprising a sequence selected from SEQ ID_No 2 or SEQ ID_No 5;
(ii) 0.01-0.04% w/w an mRNA polynucleotide comprising an open reading frame comprising a sequence selected from SEQ ID_No 3;
(iii) 0.001-0.02% w/w an mRNA polynucleotide comprising an open reading frame comprising a sequence selected from SEQ ID_No 2 or SEQ ID_No 4;
(iv) 1.5-2.5% w/w an amphipathic cell penetrating peptide comprising or consisting of the amino acid sequence WEARLARALARALARHLARALARALRACEA (SEQ ID_No 1) or a sequence with at least 80% sequence identity or homology; and
(v) 80-99% w/w trehalose;
formulated as an isotonic formulation in water with an osmolality of 290-310 mOsmol/kg.

In one embodiment the vaccine as described herein comprises:
(i) about 0.12% w/w an mRNA polynucleotides comprising open reading frames encoding a coronavirus S protein or an immunogenic fragment thereof;
(ii) about 0.03% w/w an mRNA polynucleotides comprising open reading frames encoding a coronavirus M protein or an immunogenic fragment thereof;
(iii) about 0.01% w/wan mRNA polynucleotides comprising open reading frames encoding a coronavirus E protein or an immunogenic fragment thereof;
(iv) about 2.09% w/w an amphipathic cell penetrating peptide comprising or consisting of the amino acid sequence WEARLARALARALARHLARALARALRACEA (SEQ ID_No 1) or a sequence with at least 80% sequence identity or homology; and
(v) about 97.75% w/w trehalose;
formulated as an isotonic formulation in water with an osmolality of 290-310 mOsmol/kg.

In one embodiment the vaccine as described herein comprises:
(i) 0.12% w/w an mRNA polynucleotide comprising an open reading frame comprising a sequence selected from SEQ ID_No 2 or SEQ ID_No 5;
(ii) 0.03% w/w an mRNA polynucleotide comprising an open reading frame comprising a sequence selected from SEQ ID_No 3;
(iii) 0.01% w/w an mRNA polynucleotides comprising open reading frames encoding a coronavirus E protein or an immunogenic fragment thereof;
(iv) 2.09% w/w an amphipathic cell penetrating peptide comprising or consisting of the amino acid sequence WEARLARALARALARHLARALARALRACEA (SEQ ID_No 1) or a sequence with at least 80% sequence identity or homology; and
(v) 97.75% w/w trehalose;
formulated as an isotonic formulation in water with an osmolality of 290-310 mOsmol/kg.

In one embodiment the vaccine as described herein comprises:
(i) 0.2-0.3% w/w an mRNA polynucleotides comprising open reading frames encoding a coronavirus S protein or an immunogenic fragment thereof;
(ii) 0.03-0.07% w/w an mRNA polynucleotides comprising open reading frames encoding a coronavirus M protein or an immunogenic fragment thereof;
(iii) 0.01-0.05% w/w an mRNA polynucleotides comprising open reading frames encoding a coronavirus E protein or an immunogenic fragment thereof;
(iv) 3.5-3.5% w/w an amphipathic cell penetrating peptide comprising or consisting of the amino acid sequence WEARLARALARALARHLARALARALRACEA (SEQ ID_No 1) or a sequence with at least 80% sequence identity or homology; and
(v) 94-97% w/w trehalose;
formulated as an isotonic formulation in water with an osmolality of 290-310 mOsmol/kg.

In one embodiment the vaccine as described herein comprises:
(i) 0.2-0.3% w/w an mRNA polynucleotide comprising an open reading frame comprising a sequence selected from SEQ ID_No 2 or SEQ ID_No 5;
(ii) 0.03-0.07% w/w an mRNA polynucleotide comprising an open reading frame comprising a sequence selected from SEQ ID_No 3;
(iii) 0.01-0.05% w/w an mRNA polynucleotide comprising an open reading frame comprising a sequence selected from SEQ ID_No 2 or SEQ ID_No 4;
(iv) 3.5-3.5% w/w an amphipathic cell penetrating peptide comprising or consisting of the amino acid sequence WEARLARALARALARHLARALARALRACEA (SEQ ID_No 1) or a sequence with at least 80% sequence identity or homology; and
(v) 94-97% w/w trehalose;
formulated as an isotonic formulation in water with an osmolality of 290-310 mOsmol/kg.

In one embodiment the vaccine as described herein comprises:
(i) about 0.24% w/w an mRNA polynucleotides comprising open reading frames encoding a coronavirus S protein or an immunogenic fragment thereof;
(ii) about 0.05% w/w an mRNA polynucleotides comprising open reading frames encoding a coronavirus M protein or an immunogenic fragment thereof;
(iii) about 0.03% w/wan mRNA polynucleotides comprising open reading frames encoding a coronavirus E protein or an immunogenic fragment thereof;
(iv) about 4.02% w/w an amphipathic cell penetrating peptide comprising or consisting of the amino acid sequence WEARLARALARALARHLARALARALRACEA (SEQ ID_No 1) or a sequence with at least 80% sequence identity or homology; and
(v) about 95.66% w/w trehalose;
formulated as an isotonic formulation in water with an osmolality of 290-310 mOsmol/kg.

In one embodiment the vaccine as described herein comprises:
(i) 0.24% w/w an mRNA polynucleotide comprising an open reading frame comprising a sequence selected from SEQ ID_No 2 or SEQ ID_No 5;
(ii) 0.05% w/w an mRNA polynucleotide comprising an open reading frame comprising a sequence selected from SEQ ID_No 3;
(iii) 0.03% w/w an mRNA polynucleotides comprising open reading frames encoding a coronavirus E protein or an immunogenic fragment thereof;
(iv) 4.02% w/w an amphipathic cell penetrating peptide comprising or consisting of the amino acid sequence WEARLARALARALARHLARALARALRACEA (SEQ ID_No 1) or a sequence with at least 80% sequence identity or homology; and
(v) 95.66% w/w trehalose;
formulated as an isotonic formulation in water with an osmolality of 290-310 mOsmol/kg.

In one embodiment the vaccine as described herein comprises:
(i) 0.75-0.95% w/w an mRNA polynucleotides comprising open reading frames encoding a coronavirus S protein or an immunogenic fragment thereof;
(ii) 0.1-0.3% w/w an mRNA polynucleotides comprising open reading frames encoding a coronavirus M protein or an immunogenic fragment thereof;
(iii) 0.01-0.2% w/w an mRNA polynucleotides comprising open reading frames encoding a coronavirus E protein or an immunogenic fragment thereof;
(iv) 13-15% w/w an amphipathic cell penetrating peptide comprising or consisting of the amino acid sequence WEARLARALARALARHLARALARALRACEA (SEQ ID_No 1) or a sequence with at least 80% sequence identity or homology; and
(v) 83-86% w/w trehalose;
formulated as an isotonic formulation in water with an osmolality of 290-310 mOsmol/kg.

In one embodiment the vaccine as described herein comprises:
(i) 0.75-0.95% w/w an mRNA polynucleotide comprising an open reading frame comprising a sequence selected from SEQ ID_No 2 or SEQ ID_No 5;
(ii) 0.1-0.3% w/w an mRNA polynucleotide comprising an open reading frame comprising a sequence selected from SEQ ID_No 3;
(iii) 0.01-0.2% w/w an mRNA polynucleotide comprising an open reading frame comprising a sequence selected from SEQ ID_No 2 or SEQ ID_No 4;
(iv) 13-15% w/w an amphipathic cell penetrating peptide comprising or consisting of the amino acid sequence WEARLARALARALARHLARALARALRACEA (SEQ ID_No 1) or a sequence with at least 80% sequence identity or homology; and
(v) 83-86% w/w trehalose;
formulated as an isotonic formulation in water with an osmolality of 290-310 mOsmol/kg.

In one embodiment the vaccine as described herein comprises:
(i) about 0.84% w/w an mRNA polynucleotides comprising open reading frames encoding a coronavirus S protein or an immunogenic fragment thereof;
(ii) about 0.19% w/w an mRNA polynucleotides comprising open reading frames encoding a coronavirus M protein or an immunogenic fragment thereof;
(iii) about 0.1% w/wan mRNA polynucleotides comprising open reading frames encoding a coronavirus E protein or an immunogenic fragment thereof;
(iv) about 14.16% w/w an amphipathic cell penetrating peptide comprising or consisting of the amino acid sequence WEARLARALARALARHLARALARALRACEA (SEQ ID_No 1) or a sequence with at least 80% sequence identity or homology; and
(v) about 84.7% w/w trehalose;
formulated as an isotonic formulation in water with an osmolality of 290-310 mOsmol/kg.

In one embodiment the vaccine as described herein comprises:
(i) 0.84% w/w an mRNA polynucleotide comprising an open reading frame comprising a sequence selected from SEQ ID_No 2 or SEQ ID_No 5;
(ii) 0.19% w/w an mRNA polynucleotide comprising an open reading frame comprising a sequence selected from SEQ ID_No 3;
(iii) 0.1% w/w an mRNA polynucleotides comprising open reading frames encoding a coronavirus E protein or an immunogenic fragment thereof;
(iv) 14.16% w/w an amphipathic cell penetrating peptide comprising or consisting of the amino acid sequence WEARLARALARALARHLARALARALRACEA (SEQ ID_No 1) or a sequence with at least 80% sequence identity or homology; and
(v) 84.7% w/w trehalose;
formulated as an isotonic formulation in water with an osmolality of 290-310 mOsmol/kg.

### Automated Formulation

In one embodiment, the vaccines are prepared via an automated controllable mixing system, for example an automated microfluidics system, for example Precision Nanosystems Ignite NanoAssemblr. This technology has the potential to control both the mixing rate and the mixing ratios during formulation of the nanoparticles, resulting in a reduction in Z-average particle size, resulting in an additional decrease in the polydispersity index when compared to manual formulation methods.

Microfluidics refers to the behaviour, precise control, and manipulation of fluids that are geometrically constrained to a small scale (typically sub-millimetre) at which surface forces dominate volumetric forces.

In one embodiment the vaccines described herein are prepared via an automated controlled mixing system.

In one embodiment there is provided a method of preparing a nanoparticle formulation which comprises formulating a solution of an mRNA polynucleotide comprising an open reading frame encoding a coronavirus antigen or an immunogenic fragment thereof; with an amphipathic cell penetrating peptide comprising or consisting of the amino acid sequence WEARLARALARALARHLARALARALRACEA (SEQ ID_No 1) or a sequence with at least 80% sequence identity or homology in an automated controlled mixing system, particularly an automated microfluidics system.

In one embodiment there is provided a method of preparing a nanoparticle formulation which comprises formulating a solution of an mRNA polynucleotide comprising an open reading frame encoding a coronavirus antigen or an immunogenic fragment thereof; with an amphipathic cell penetrating peptide comprising or consisting of the amino acid sequence WEARLARALARALARHLARALARALRACEA (SEQ ID_No 1) or a sequence with at least 80% sequence identity or homology in an automated controlled mixing system wherein the flow rate ratio of mRNA:peptide is 1:3.

In one embodiment there is provided a method of preparing a nanoparticle formulation which comprises formulating a solution of an mRNA polynucleotide comprising an open reading frame encoding a coronavirus antigen or an immunogenic fragment thereof; with an amphipathic cell penetrating peptide comprising or consisting of the amino acid sequence WEARLARALARALARHLARALARALRACEA (SEQ ID_No 1) or a sequence with at least 80% sequence identity or homology in an automated controlled mixing system wherein the flow rate ratio of mRNA:peptide is about 1:3.

In one embodiment there is provided a method of preparing a nanoparticle formulation which comprises formulating a solution of an mRNA polynucleotide comprising an open reading frame encoding a coronavirus antigen or an immunogenic fragment thereof; with an amphipathic cell penetrating peptide comprising or consisting of the amino acid sequence WEARLARALARALARHLARALARALRACEA (SEQ ID_No 1) or a sequence with at least 80% sequence identity or homology in an automated controlled mixing system wherein the flow rate ratio of mRNA:peptide is 1:5 to 5:1.

In one embodiment there is provided a method of preparing a nanoparticle formulation which comprises formulating a solution of an mRNA polynucleotide comprising an open reading frame encoding a coronavirus antigen or an immunogenic fragment thereof; with an amphipathic cell penetrating peptide comprising or consisting of the amino acid sequence WEARLARALARALARHLARALARALRACEA (SEQ ID_No 1) or a sequence with at least 80% sequence identity or homology in an automated controlled mixing system wherein the flow rate ratio of mRNA:peptide is about 1:5 to 5:1.

### Formulation

Nanoparticles may be formed by self-assembly by adding the mRNA and the amphipathic cell penetrating peptide together in ultrapure water with instantaneous formulation occurring. The resulting vaccine may be lyophilised for transport and storage, and then rehydrate in water for use.

The vaccine described herein may be employed in various routes of administration, for example oral, nasal, inhaled, rectal, topically, percutaneous, intravitreal, intravenous, intramuscular, or intradermal administration, particularly intradermal or intramuscular administration. Intradermal and intramuscular routes of administration may maximise delivery to dendritic cells which are highly prevalent near the skin surface.

In one embodiment, the vaccine of the present disclosure may be employed in an injectable formulation, for example an intradermal injection.

In one embodiment, the vaccine of the present disclosure may be employed in an injectable formulation, for example an intramuscular injection.

In one embodiment, the vaccine of the present disclosure may be employed in an injectable formulation, for example an intravenous injection.

In one embodiment, the vaccine of the present disclosure may be employed in an injectable formulation, for example an intradermal patch. The surface of an intradermal patch is covered in tiny microneedles which dissolve in the body. The patch can be applied painlessly, like a plaster, and allows the vaccine to quickly overcome the outer skin barrier and be delivered straight into the area with most dendritic cells.

### Dose

The exact amount required for effective vaccination will vary from subject to subject, depending on the species, age, and general condition of the subject, the severity of the disease, the particular composition, its mode of administration, its mode of activity, the desired effect and the like. Vaccines compositions are typically formulated in dosage unit form for ease of administration and uniformity of dosage. It will be understood, however, that the total dose of vaccine compositions may be decided by the attending physician within the scope of sound medical judgment. Vaccines may be administered more than once, for example two or three times on separate occasions, in order to maximise the effect.

In one embodiment the vaccine may be administered at a dosage of between 0.1 μg/kg and 500 μg/kg to the subject.

In one embodiment the vaccine may be administered at a dosage of between 1 μg/kg and 500 μg/kg to the subject.

In one embodiment, the vaccine described herein comprises 10-250 μg of mRNA polynucleotide.

In one embodiment, the vaccine described herein comprises 10-150 μg of mRNA polynucleotide.

In one embodiment, the vaccine described herein comprises about 100 μg mRNA polynucleotide.

In one embodiment, the vaccine described herein comprises 100 μg mRNA polynucleotide.

In one embodiment, the vaccine is administered twice.

In one embodiment, the vaccine is administered three times.

In one embodiment, the vaccine is administered more than three times.

In one embodiment, the vaccine is administered 1-10 times.

In one embodiment, the vaccine is administered once, and then additional (booster) doses are administered as needed.

In one embodiment, the vaccine is administered twice, and then additional (booster) doses are administered as needed.

In one embodiment, the vaccine described herein comprises 10-250 μg mRNA polynucleotide and is administered twice, and then additional (booster) doses are administered as needed.

In one embodiment, the vaccine described herein comprises 10-150 μg mRNA polynucleotide and is administered twice, and then additional (booster) doses are administered as needed.

In one embodiment, the vaccine described herein comprises at least two mRNA polynucleotides comprising open reading frames and encoding a coronavirus antigen or an immunogenic fragment thereof, wherein the dosage for the vaccine is a combined therapeutic dosage, and wherein the dosage of each individual mRNA polynucleotide is a sub therapeutic dosage.

### Age

In one embodiment, the vaccine described herein may be administered to a subject under 5 years old.

In one embodiment, the vaccine described herein may be administered to a subject aged 5-11 years old.

In one embodiment, the vaccine described herein may be administered to a subject aged 9-45 years old.

In one embodiment, the vaccine described herein may be administered to a subject aged 12-15 years old.

In one embodiment, the vaccine described herein may be administered to a subject aged 16 or 17 years old.

In one embodiment, the vaccine described herein may be administered to a subject aged 17 years or younger.

In one embodiment, the vaccine described herein may be administered to a subject aged 18 years or older.

In one embodiment, the vaccine described herein may be administered to a subject aged 60 years or older.

### Vaccine Effectiveness

Vaccine efficacy may be assessed using standard analyses (see, e.g., Weinberg et al., J Infect Dis. 2010 Jun. 1:201(11):1607-10). For example, vaccine efficacy may be measured by double-blind, randomized, clinical controlled trials. Vaccine efficacy may be expressed as a proportionate reduction in disease attack rate (AR) between the unvaccinated (ARU) and vaccinated (ARV) study cohorts and can be calculated from the relative risk (RR) of disease among the vaccinated group with use of the following formulas:

$$Efficacy = ( ARU - ARV ) / ARU \times 100 ;$$

$$and$$

$$Efficacy = ( 1 - RR ) \times 100.$$

Likewise, vaccine effectiveness may be assessed using standard analyses (see. e.g., Weinberg et al., J Infect Dis. 2010 Jun. 1; 201(11):1607-10). Vaccine effectiveness is an assessment of how a vaccine (which may have already proven to have high vaccine efficacy) reduces disease in a population. This measure can assess the net balance of benefits and adverse effects of a vaccination program, not just the vaccine itself, under natural field conditions rather than in a controlled clinical trial. Vaccine effectiveness is proportional to vaccine efficacy (potency) but is also affected by how well target groups in the population are immunized, as well as by other non-vaccine-related factors that influence the ‘real-world’ outcomes of hospitalizations, ambulatory visits, or costs. For example, a retrospective case control analysis may be used, in which the rates of vaccination among a set of infected cases and appropriate controls are compared. Vaccine effectiveness may be expressed as a rate difference, with use of the odds ratio (OR) for developing infection despite vaccination:

$$Effectiveness = ( 1 - OR ) \times 100.$$

In one embodiment, the vaccine immunizes the subject for up to 2 years.

In one embodiment, the vaccine immunizes the subject for more than 2 years, more than 3 years, more than 4 years, or for 5-10 years.

An “effective amount” of a vaccine may be determined by the target tissue, target cell type, means of administration, physical characteristics of the components of the vaccine, and other determinants. In general, an effective amount of the vaccine composition provides an induced or boosted immune response as a function of antigen production in the cell. Increased antigen production may be demonstrated by increased cell transfection, increased protein translation, decreased nucleic acid degradation, or altered antigen specific immune response of the host cell. In one embodiment an “effective amount” is a therapeutically effective amount.

### Charge Density

The mean surface charge density of nanoparticles may be a contributing factor to their toxicity by promoting oxidative stress mechanisms which in turn can promote mitochondrial dysfunction and viability loss.

The charge density may be measured by polyelectrolytic titration using methods described in Ritz et al, Biomacromolecules. 2015 Apr. 13; 16(4):1311-21. doi: 10.1021/acs.biomac.5b00108. Epub 2015 Apr. 3 and Weiss et al, J Nanobiotechnology. 2021 Jan. 6; 19(1):5. doi: 10.1186/s12951-020-00747-7.

Polyelectrolytic titration may be performed using poly(acrylic acid) (PAA) 0.01 M at pH 7.4 and addition of PAA to nanoparticles and measuring the charge creates a sigmoidal curve of which the volume (V) can be derived from the equivalence point. In conjunction, the mass of peptide in the system (w) and concentration of PAA (0.01 M) used, is used to calculate the average charge density of the nanoparticle (Qₑₖ).

In one embodiment, the mean surface charge density of the nanoparticles comprising vaccines as described herein is ≤2 μmol/mg at 20° C. Mean surface charge density figures were measured using an Orion STAR® multi parameter bench meter.

In one embodiment, the mean surface charge density of the nanoparticles comprising vaccines as described herein is ≤1.5 μmol/mg at 20° C.

In one embodiment, the mean surface charge density of the nanoparticles comprising vaccines as described herein is about 1 μmol/mg at 20° C.

In one embodiment, the mean surface charge density of the nanoparticles comprising vaccines as described herein is 1 μmol/mg at 20° C.

### Uses

In one embodiment there is provided a vaccine as described herein for use as a medicament.

In one embodiment there is provided the use of a vaccine as described herein as a medicament.

In one embodiment there is provided a vaccine as described herein for use in therapy.

As used herein, the terms “treatment” and “treat” refer to preventing, reversing, alleviating, delaying the onset of, or inhibiting the progress of a disease or disorder, or one or more symptoms thereof, as described herein. In one embodiment, treatment may be conducted before infection has occurred. In one embodiment, treatment may be conducted in subjects exposed to infection. In one embodiment, treatment may be conducted in subjects at risk of infection. In one embodiment, treatment may be conducted after one or more symptoms have developed. In other embodiments, treatment may be conducted in the absence of symptoms. For example, treatment may be conducted to a susceptible individual prior to the onset of symptoms (e.g. in light of a history of symptoms and/or in light of genetic or other susceptibility factors and/or indicative diagnostic tests). Treatment may also be continued after symptoms have resolved, for example to present or delay their recurrence.

In one embodiment there is provided a vaccine as described herein for use as a vaccine.

In one embodiment there is provided a vaccine as described herein for use in creating, maintaining or restoring antigenic memory to a virus strain.

In one embodiment there is provided a vaccine as described herein for use in inducing an antigen specific immune response in a subject.

In one embodiment there is provided a vaccine as described herein for use in delivering mRNA comprising an open reading frame encoding a coronavirus antigen or an immunogenic fragment thereof, into a cell.

In one embodiment there is provided a vaccine as described herein for use in delivering mRNA comprising an open reading frame encoding a coronavirus antigen or an immunogenic fragment thereof, to a target tissue of the immune system, particularly the lymph nodes, spleen and/or bone marrow.

In one embodiment there is provided a vaccine as described herein for use in producing a coronavirus antigen or an immunogenic fragment thereof, in a cell, tissue or organism.

In one embodiment there is provided a vaccine as described herein for use in the treatment of coronavirus infection.

In one embodiment there is provided a vaccine as described herein for use in the treatment of SARS-CoV-2 infection.

In one embodiment there is provided a vaccine as described herein for use in the treatment of COVID-19. COVID-19 has a number of different symptoms including, but not limited to: fever, cough, tiredness, loss of taste or smell, shortness of breath or difficulty breathing, muscle aches, chills, sore throat, runny nose, headache, chest pain, conjunctivitis, nausea, vomiting, diarrhoea, multisystem inflammatory syndrome, and/or rash. The severity of COVID-19 symptoms can range from very mild/asymptomatic to severe. COVID-19 symptoms can persist for more than four weeks after diagnosis (post- or long-COVID).

### Pharmaceutical Compositions

In one embodiment there is provided a pharmaceutical composition which comprises a vaccine as described herein for use in an intradermal injection.

In one embodiment there is provided a pharmaceutical composition which comprises a vaccine as described herein for use in an intramuscular injection.

In one embodiment there is provided a pharmaceutical composition which comprises a vaccine as described herein for use as a vaccine.

In one embodiment there is provided a pharmaceutical composition which comprises a vaccine as described herein for use in creating, maintaining or restoring antigenic memory to a virus strain.

In one embodiment there is provided a pharmaceutical composition which comprises a vaccine as described herein for use in inducing an antigen specific immune response in a subject.

In one embodiment there is provided a pharmaceutical composition which comprises a vaccine as described herein for use in delivering mRNA comprising an open reading frame encoding a coronavirus antigen or an immunogenic fragment thereof, into a cell.

In one embodiment there is provided a pharmaceutical composition which comprises a vaccine as described herein for use in delivering mRNA comprising an open reading frame encoding a coronavirus antigen or an immunogenic fragment thereof, to a target tissue of the immune system, particularly the lymph nodes, spleen and/or bone marrow.

In one embodiment there is provided a pharmaceutical composition which comprises a vaccine as described herein for use in producing a coronavirus antigen or an immunogenic fragment thereof, in a cell, tissue or organism.

In one embodiment there is provided a pharmaceutical composition which comprises a vaccine as described herein for use in the treatment of coronavirus infection.

In one embodiment there is provided a pharmaceutical composition which comprises a vaccine as described herein for use in the treatment of SARS-CoV-2 infection.

In one embodiment there is provided a pharmaceutical composition which comprises a vaccine as described herein for use in the treatment of COVID-19.

### Methods of Treatment

In one embodiment there is provided a method of intradermal injection which comprises administering a vaccine as described herein.

In one embodiment there is provided a method of intravenous injection which comprises administering a vaccine as described herein.

In one embodiment there is provided a method of intramuscular injection which comprises administering a vaccine as described herein.

In one embodiment there is provided a method of vaccination in a warm-blooded animal, such as man, which comprises administering to said animal an effective amount of a vaccine as described herein.

In one embodiment there is provided a method of creating, maintaining or restoring antigenic memory to a virus strain in a warm-blooded animal, such as man, which comprises administering to said animal an effective amount of a vaccine as described herein.

In one embodiment there is provided a method of inducing an antigen specific immune response in a subject in a warm-blooded animal, such as man, which comprises administering to said animal an effective amount of a vaccine as described herein.

In one embodiment there is provided a method of delivering mRNA comprising an open reading frame encoding a coronavirus antigen or an immunogenic fragment thereof, into a cell in a warm-blooded animal, such as man, which comprises administering to said animal an effective amount of a vaccine as described herein.

In one embodiment there is provided a method of delivering mRNA comprising an open reading frame encoding a coronavirus antigen or an immunogenic fragment thereof, to a target tissue of the immune system, particularly the lymph nodes, spleen and/or bone marrow in a warm-blooded animal, such as man, which comprises administering to said animal an effective amount of a vaccine as described herein.

In one embodiment there is provided a method of producing a coronavirus antigen or an immunogenic fragment thereof, in a cell, tissue or organism in a warm-blooded animal, such as man, which comprises administering to said animal an effective amount of a vaccine as described herein.

In one embodiment there is provided a method of treating coronavirus infection in a warm-blooded animal, such as man, which comprises administering to said animal an effective amount of a vaccine as described herein.

In one embodiment there is provided a method of treating SARS-CoV-2 infection in a warm-blooded animal, such as man, which comprises administering to said animal an effective amount of a vaccine as described herein.

In one embodiment there is provided a method of treating COVID-19 in a warm-blooded animal, such as man, which comprises administering to said animal an effective amount of a vaccine as described herein.

### Use of a Vaccine

In one embodiment there is provided the use of a vaccine as described herein for the manufacture of a medicament for intradermal injection.

In one embodiment there is provided the use of a vaccine as described herein for the manufacture of a medicament for intramuscular injection.

In one embodiment there is provided the use of a vaccine as described herein for the manufacture of a medicament for vaccination.

In one embodiment there is provided the use of a vaccine as described herein for the manufacture of a medicament for creating, maintaining or restoring antigenic memory to a virus strain.

In one embodiment there is provided the use of a vaccine as described herein for the manufacture of a medicament for inducing an antigen specific immune response in a subject.

In one embodiment there is provided the use of a vaccine as described herein for the manufacture of a medicament for delivering mRNA comprising an open reading frame encoding a coronavirus antigen or an immunogenic fragment thereof, into a cell.

In one embodiment there is provided the use of a vaccine as described herein for the manufacture of a medicament for delivering mRNA comprising an open reading frame encoding a coronavirus antigen or an immunogenic fragment thereof, to a target tissue of the immune system, particularly the lymph nodes, spleen and/or bone marrow.

In one embodiment there is provided the use of a vaccine as described herein for the manufacture of a medicament for producing a coronavirus antigen or an immunogenic fragment thereof, in a cell, tissue or organism.

In one embodiment there is provided the use of a vaccine as described herein for the manufacture of a medicament for the treatment of coronavirus infection.

In one embodiment there is provided the use of a vaccine as described herein for the manufacture of a medicament for the treatment of SARS-CoV-2 infection.

In one embodiment there is provided the use of a vaccine as described herein for the manufacture of a medicament for the treatment of COVID-19.

### Kits

In one embodiment there is provided a kit comprising:
a) a vaccine as described herein;
b) container means for containing said vaccine.

In one embodiment there is provided a kit comprising:
a) a vaccine as described herein;
b) container means for containing said vaccine; and optionally
c) instructions for use.

### DESCRIPTION OF THE FIGURES

FIG. 1 shows a representative size distribution spectrum of Compound 008 nanoparticles.

FIG. 2 shows SARS-CoV-2 protein production by Compound 008 following transfection into NCTC-929 mouse fibroblast cells (analysed by in-cell ELISA).

FIG. 3 summarises the weights of BALBc mice treated with the indicated dose of Compound 001, 002, 003 or 004. Mice were vaccinated intradermally via the left ear with the indicated dose of Compound 001, 002, 003 or 004 on Days 0 and 21, and were weighed every day until termination of the experiment (Day 42).

FIG. 4 summarises Spike-, Membrane- and Envelope-specific IgG antibodies present in the sera of vaccinated mice. Standard curves were generated for Spike, Membrane and Envelope using commercially-available IgG, and optical density values from these curves were used to estimate the concentration of antigen-specific antibodies in the sera of vaccinated mice.

FIG. 5 summarises the creatinine concentration and AST/ALT activities in the sera of vaccinated mice.

FIG. 6 summarises the weight of hamsters vaccinated with Compounds 001, 005, 006, 007 and 008 pre- and post-challenge with SARS-CoV-2. Hamsters were vaccinated on Days 0 and 21, and challenged with virus on Day 42.

FIG. 7 the replication-competent titers of SARS-CoV-2 in the throat swabs (Days 42-46), nasal turbinates and lungs (Day 46) of vaccinated and challenged hamsters.

FIG. 8 summarises the titers of virus (wild-type) neutralising antibodies in the serum of hamsters vaccinated with Compounds 001, 005, 006, 007 and 008.

FIG. 9 summarises the titers of virus (Omicron BA.1) neutralising antibodies in the serum of hamsters vaccinated with Compounds 001, 005, 006, 007 and 008.

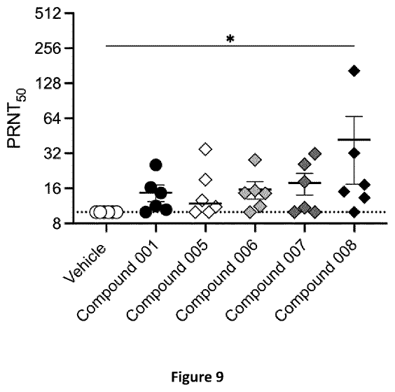

FIG. 10 summarises the weights of C57BL/6 mice following vaccination with 3.4 μg, 6.7 μg or 26.9 μg of Compound 008.

FIG. 11 summarises the antigen-specific, cell-mediated immune response in splenocytes of C57BL/6 mice that were vaccinated with Compound 008. Mice were vaccinated intradermally via the left ear or intramuscularly via the thigh with the indicated dose of Compound 008 on Days 0 and 21, and sacrificed on Day 42. Single cell splenocyte suspensions were prepared, and the ability of the cells to secrete IFNγ or IL-2 following in vitro stimulation with overlapping peptides corresponding to the full length sequences of Spike, Membrane or Envelope were assessed by ELISpot.

FIG. 12 summarises the antibody-mediated immune response in BALB/c mice vaccinated intradermally or intramuscularly with Compound 008.

FIG. 13 summarises the IgG2:IgG1 ratio in the serum of vaccinated mice.

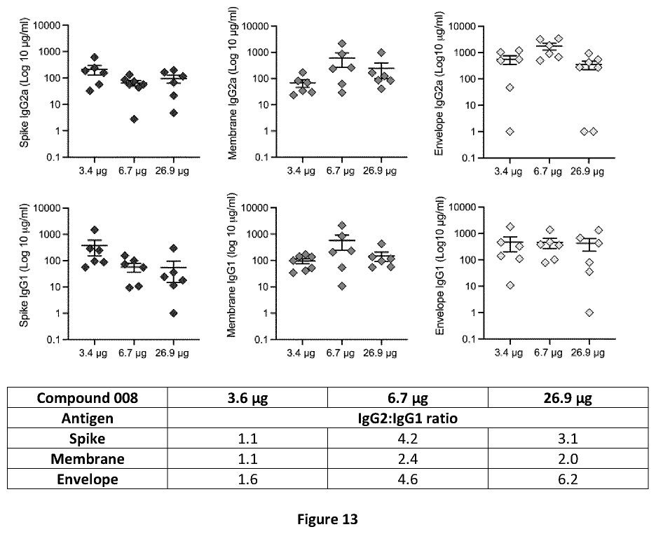

FIG. 14 summarises summarises the antigen-specific, cell-mediated immune response in splenocytes of Sprague Dawley rats that were vaccinated with Compound 008. Rats were vaccinated intradermally via the left ear with the indicated dose of Compound 008 on Days 0 and 21, and sacrificed on Day 42. Single cell splenocyte suspensions were prepared, and the ability of the cells to secrete IFNγ or IL-2 following in vitro stimulation with overlapping peptides corresponding to the full length sequences of Spike, Membrane or Envelope were assessed by ELISpot.

FIG. 15 summarises summarises the antibody-mediated immune response in Sprague Dawley rats vaccinated intradermally with Compound 008.

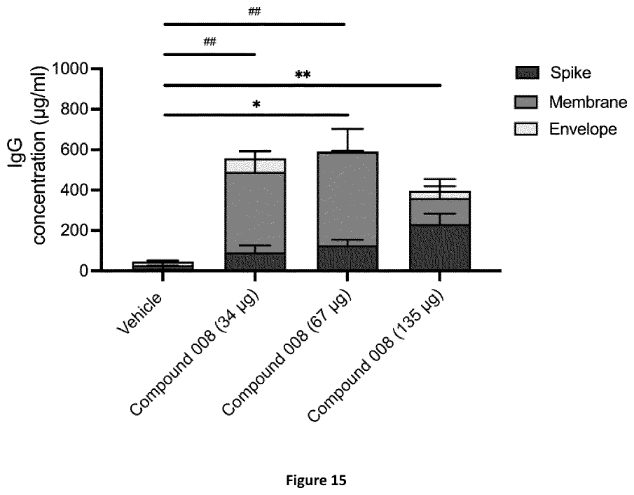

FIG. 16 summarises the concentrations of serum biomarkers associated with toxicity in serum of Sprague Dawley rats vaccinated with Compound 008.

FIG. 17 summarises the concentrations of serum biomarkers associated with toxicity in serum of C57BL/6 mice vaccinated with Compound 009.

FIG. 18 summarises the histopathological assessment of selected organs from BALB/c mice vaccinated intradermally with Compound 009.

FIG. 19 summarises the Spike-specific IgG response in BALB/c mice vaccinated intradermally with Compound 009.

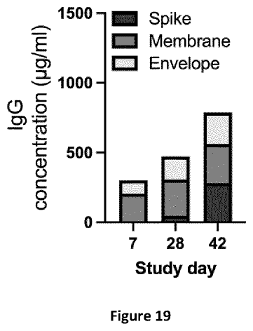

FIG. 20 demonstrates the encapsulation of mRNA by RALA in Compound 008.

### EXAMPLES

### General Experimental

In the Example section, the following applies:
RALA refers to the peptide WEARLARALARALARHLARALARALRACEA (SEQ ID_No 1) and was obtained from Biomatik®, Canada in lyophilised powder form;
mRNA (Spike) refers to a mRNA with 5′ and 3′ untranslated regions, a poly-A tail comprising 120 adenosines and a basic open reading frame sequence of SEQ ID_No 2, with a 5′ CleanCap purchased from Trilink Biotechnologies®;
mRNA (SpikeZA) refers to a mRNA with 5′ and 3′ untranslated regions, a poly-A tail comprising 120 adenosines and a basic open reading frame sequence of SEQ ID_No 5, with a 5′ CleanCap purchased from Trilink Biotechnologies®;
mRNA (Membrane) refers to a mRNA with 5′ and 3′ untranslated regions, a poly-A tail comprising 120 adenosines and a basic open reading frame sequence of SEQ ID_No 3, with a 5′ CleanCap or ARCA cap purchased from Trilink Biotechnologies®;
mRNA (Envelope) refers to a mRNA with 5′ and 3′ untranslated regions, a poly-A tail comprising 120 adenosines and a basic open reading frame sequence of SEQ ID_No 4, with a 5′ CleanCap purchased from Trilink Biotechnologies®;
All mRNAs were purchased from Trilink Biotechnologies® and were used without further modification;
NCTC-929 cells were obtained from American Type Culture Collection (ATCC);
BALB/c and C57BL/6 mice were obtained from Envigo®;
NCTC-929 mouse fibroblasts were obtained from ATCC; and
Creatinine, AST and ALT assays were from Abcam.

### Example 1: Preparation of RALA Peptide

RALA was reconstituted with molecular grade water to a desired concentration, aliquoted and stored at −20° C. until further use. An aliquot was taken as needed and defrosted on ice. Aliquots were not re-frozen once they had been defrosted.

### Example 2: Nanoparticle Formulation

RALA/mRNA nanoparticles were formulated at N:P ratio 9:1 by use of an automated microfluidics system (e.g. Precision Nanosystems Ignite NanoAssemblr). Two solutions containing mRNA (0.4 μg/μl) and RALA (1.68 μg/μl) were loaded into syringes, and the syringes subsequently loaded into the microfluidics system. Nanoparticles were created using a Total Flow Rate (TFR) of 10 mL/min, and a Flow Rate Ratio (FRR) of 1:3 (mRNA:RALA). The resultant solution from the system contained nanoparticles at the concentration of 0.1 μg/μl of mRNA.

The following compounds were prepared.

| | | | Antigen |
|---|---|---|---|
| Com- | | Antigen | mRNA |
| pound | | mRNA % | copy number |
| no | mRNA | w/w ratio | ratio |
| 001 | Spike | — | — |
| 002 | Membrane | — | — |
| 003 | Envelope | — | — |
| 004 | Spike:Membrane:Envelope | 33:33:33 | 7:32:61 |
| 005 | Spike:Membrane | 80:20 | 47:53 |
| 006 | Spike:Envelope | 80:20 | 32:68 |
| 007 | Spike:Membrane:Envelope | 67:17:17 | 24:26:50 |
| 008 | Spike:Membrane:Envelope | 74:17:9 | 33:33:33 |
| 009 | SpikeZA:Membrane:Envelope | 74:17:9 | 33:33:33 |

### Example 3: Lyophilisation of RALA/mRNA Nanoparticles

200 μL of RALA/mRNA nanoparticles was transferred into a 2 mL lyophilisation vial. A cryoprotectant, trehalose (20% w/v), was added to the nanoparticle solution in the vial, such that the volume added was equal to half the final reconstitution volume ensuring that once reconstituted in water following lyophilisation, the resultant solution contained 10% w/v cryoprotectant. Following the addition of cryoprotectant, a rubber lyophilisation vial stopper was partially placed on the vial, such that the air-flow notches were still functional to facilitate sublimation during the lyophilisation process. Vials were subsequently loaded into a programmable freeze dryer (e.g. SP Scientific AdVantage Pro) according to the following lyophilisation procedure:

Thermal Treatment

| | | Shelf Temp. | Ramp Time | Hold Time |
|---|---|---|---|---|
| 1 | Step | [° C.] | [min] | [min] |
| | 1 | 5 | 60 | 10 |
| | 2 | −35 | 90 | 0 |

| | Drying Steps | | | | |
|---|---|---|---|---|---|
| | | Shelf Temp. | Ramp Time | Hold Time | Vacuum |
| 2 | Step | [° C.] | [min] | [min] | [mTorr] |
| | 3 | −35 | 0 | 180 | 120 |
| | 4 | −30 | 60 | 0 | 190 |
| | 5 | −30 | 0 | 180 | 190 |
| | 6 | −25 | 60 | 0 | 190 |
| | 7 | −25 | 0 | 180 | 190 |
| | 8 | 20 | 120 | 0 | 190 |
| | 9 | 20 | 0 | 360 | 190 |

| | |
|---|---|
| 3 | Storage |

| | | |
|---|---|---|
| Shelf Temp | 20 | ° C. |
| Vacuum | 50 | mTorr |

| | | |
|---|---|---|
| 4 | Backfilling | None |

| | | |
|---|---|---|
| 5 | Stoppering | Vials Stoppered under Vacuum |

In the following examples, the compounds of Example 2 were lyophilised with trehalose and reconstituted according to the procedure of Example 3.

### Example 4: Nanoparticle Size Analysis

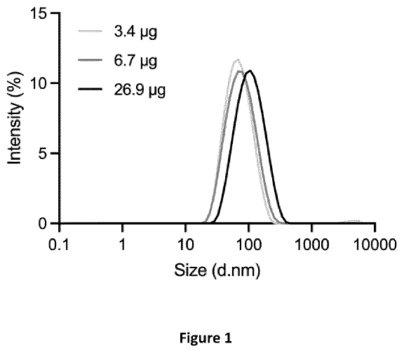

Z-Average particle size measurements and polydispersity (PdI) of RALA/mRNA nanoparticles were performed using Dynamic Light Scattering (DLS) in order to obtain particle size and charge distributions. Surface charge measurements of the RALA nanoparticles were determined by Laser Doppler Velocimetry. The nanoparticles were made up using at least 1 μg of mRNA in each sample. Nanoparticles were analysed on a Zetasizer-Nano-ZS (Malvern Instruments) with DTS software (Malvern Instruments, UK) and the results for Compound 008 is shown in FIG. 1.

### Example 5: Assessment of In Vitro Functionality of Compound 008

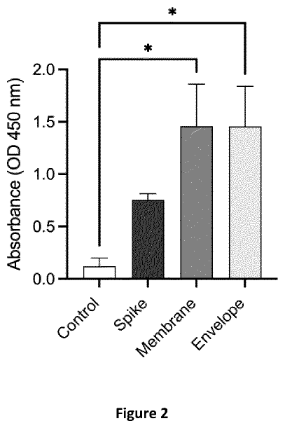

Initial studies were undertaken to determine the functionality of mRNAs by assessing protein expression in vitro. Compound 004 was assessed for Spike, Membrane and Envelope expression in NCTC-929 mouse fibroblasts by in-cell ELISA. Cells plated in 96 well plates were transfected with Compound 008, and assessed for Spike, Membrane and Envelope protein expression 24 h later. Following fixation and permeabilization, cells were probed with an anti-Spike, anti-Membrane or anti-Envelope primary antibody, excess antibody was removed by washing with PBS/0.05% Tween 20, and cells were probed with an anti-rabbit secondary antibody conjugated to horseradish peroxidase. Protein expression was detected using TMB and Stop solution (standard ELISA reagents), and optical density (OD) at 450 nm was measured using a Molecular Devices SpectraMax iD3 plate reader (FIG. 2).

### Example 6: Assessment of Antibody-Mediated Immunogenicity and Safety of Compound 001, 002, 003 and 004

BALB/c mice were injected intradermally with 20 μg of Compound 001, 002, 003 or 004 on Days 0 and 21 with sacrifice at Day 42. Blood from the mice was removed by cardiac puncture, transferred to sterile 1.5 ml centrifuge tubes and allowed to clot for 30 min. Serum was separated from the clot by centrifuging the tubes at 10,000×g for 10 min, before transferring to a clean 1.5 ml centrifuge tubes, and stored at −20° C.

Sera from vaccinated mice were investigated for presence of antigen (ie Spike, Membrane or Envelope)-specific antibodies by ELISA. Nunc Maxisorp 96 well plates were coated with recombinant Spike, Membrane or Envelope (as appropriate) by addition of 50 μl of recombinant protein at 2 μg/ml (in ELISA coating buffer), followed by a two-step incubation (1 h at 37° C. followed by overnight at 4° C.). Non-specific binding of serum components to wells was minimised by incubation with blocking solution (0.1% w/v BSA in PBS) for 1 h at room temperature. Samples were diluted as appropriate (eg 1:10, 1:100, 1:1000 in 0.1% w/v BSA in PBS), and standard curves were constructed (10000, 3333.3, 1111.1, 370.4, 123.5, 41.2 & 13.7 pg/ml) using commercial antibodies against Spike (Bio-Techne), Membrane (Bio-Techne) and Envelope (Abcam). Blocked wells were probed with diluted sera (overnight, 4° C.), and following three washes with PBS/0.01% Tween 20, wells were probed with a goat anti-mouse IgG (Abcam) diluted 1:10000 in 0.1% w/v BSA in PBS. Wells were washed three more times with PBS/0.01% Tween 20 before addition of 100 μl/well of TMB substrate. Following 15 min colour development incubation, an equal quantity of stop solution was added and absorbance read at 450 nm using a Molecular Devices SpectraMax iD3 plate reader.

Serum creatinine levels in vaccinated mice were measured as a marker for kidney function (Blood creatinine levels increase only in cases of significant (>75%) damage to nephrons). Liver function assays were similarly run interrogating Aspartate aminotransferase (AST) and Alanine transaminase (ALT). All colorimetric assays were used as recommended by the manufacturer (Abcam), whereby standard curves were generated, and serum levels of each marker were estimated by extrapolation from the respective standard curves.

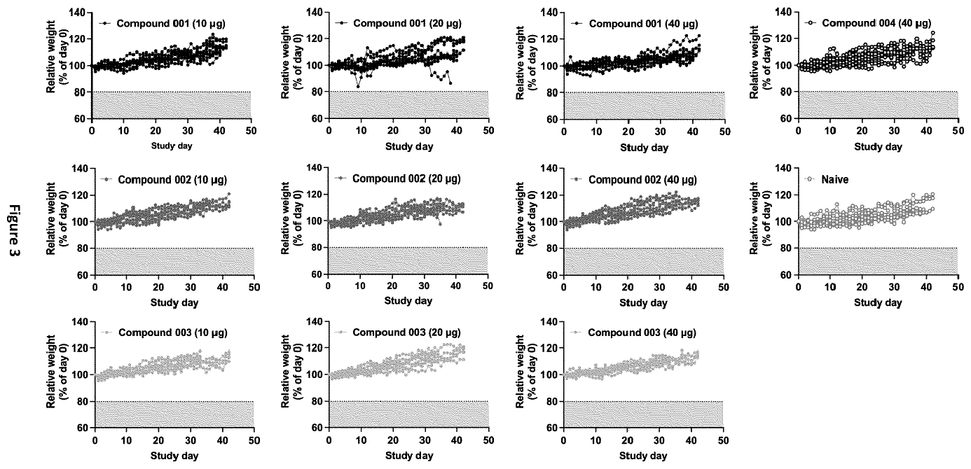

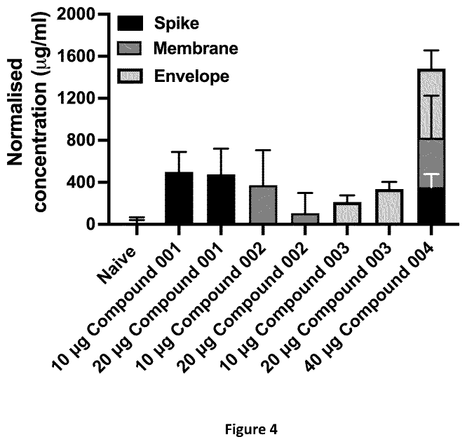

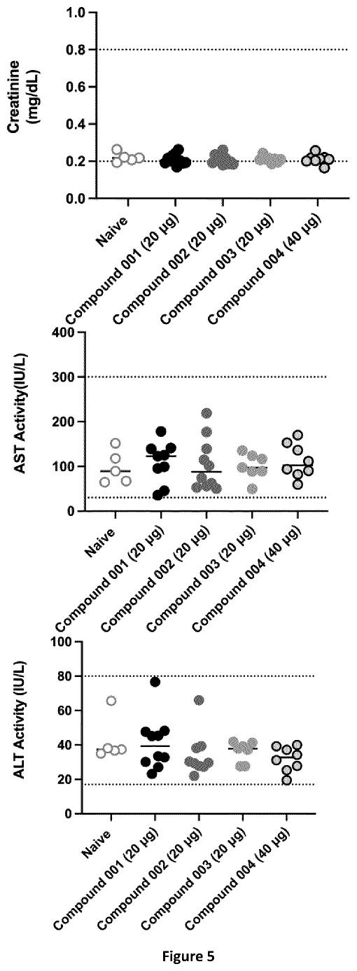

The results of Example 6 are shown in FIGS. 3-5.

### Example 7: Assessment of Efficacy of Compound 001, 005, 006, 007 & 008 in Hamster Challenge Study

Unless otherwise stated, all hamster studies, were conducted at Viroclinics Xplore (Schaijk, The Netherlands). Female Syrian Golden hamsters (Janvier, France) of 7 weeks age were randomly allocated to treatment groups. Hamsters were vaccinated intradermally via the ear on Day 0 and Day 21 while under isoflurane anaesthesia. On Day 42, hamsters were challenged intranasally with 102 TCID₅₀/dose of SARS-CoV-2 (BetaCoV/Munich/BavPat1/2020) in a total inoculum volume of 100 μL.

On Day 0 and Day 21 (pre-vaccination), as well as Day 42 (pre-challenge), approximately 250 μL of blood samples were taken via the medial canthus of the eye and processed to serum as previously described. On Days 42 to 46, throat swabs were taken daily to sample the respiratory tract. The samples were collected in virus transport medium, aliquoted and stored. Upon necropsy, tissue samples were collected and stored in 10% formalin for histopathology or frozen for virological analysis. For virological analysis, tissue samples were weighed, homogenised in infection medium, and briefly centrifuged before titration. On Day 46, following a final throat swab and blood sampling, hamsters were euthanised by abdominal exsanguination, and the lung and nasal turbinates were isolated.

The ability of generated antibodies to bind SARS-CoV-2 was assessed by virus neutralisation assay. Serum samples were heat-inactivated for 30 min at 56° C. Subsequently, 102 infectious units were incubated with serial dilutions of the serum. After a 60±15 min incubation period of the virus/serum mixture at room temperature (RT), 100 μL of the mixture were added to a confluent monolayer of Vero-E6 cells (ATCC, UK, Cat no. C100) for a period of 60±15 min at 37° C. The inoculum was removed, and fresh medium with 1.6% carboxymethylcellulose was added. The plates were then incubated for a total duration of 24±2 h at 37° C. Cells were fixed, and virus-infected cells immunostained with a monoclonal antibody which targets the SARS-CoV-2 nucleocapsid protein, followed by a secondary goat anti-mouse IgG peroxidase conjugate and TrueBlue substrate. This forms a blue precipitate on nucleocapsid protein positive cells. Images of all wells were acquired by a CTL ImmunoSpot analyser equipped with software to quantitate the nucleocapsid-positive cells. The 80% (ancestral strain) or 50% (Omicron BA.1 variant) neutralisation (MN80 or MN50) titers were calculated according to the method of Spearman-Karber.

The levels of replication-competent virus were assessed using a plaque formation assay in Vero E6 cells. Quadruplicate 10-fold serial dilutions were used to determine the virus titres in confluent layers of Vero E6 cells (ATCC, UK, Cat no. C100). Samples (throat swabs and lung/nasal turbinate homogenates) were serially diluted and incubated on Vero E6 monolayers for 1 h at 37° C. The Vero E6 monolayers were washed and incubated for 4-6 days at 37° C., after which plates were scored using the viability marker WST8. The viral titers (TCID50) were calculated using the method of Spearman-Karber.

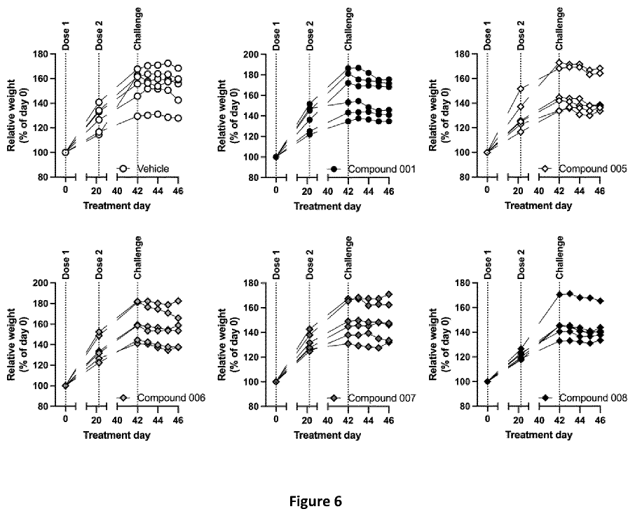

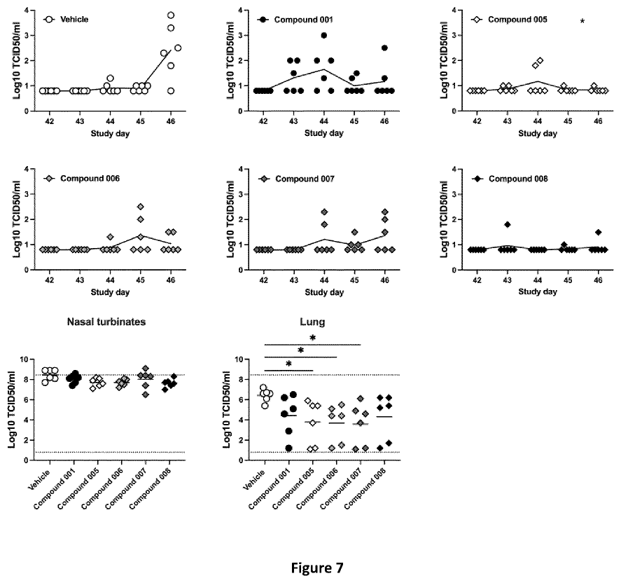

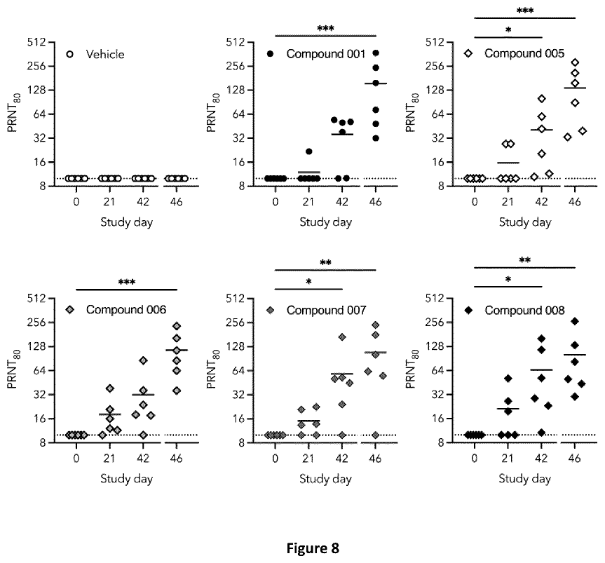

The results of Example 7 are shown in FIGS. 6, 7, 8 & 9.

### Example 8: Assessment of Immunogenicity of Compound 008 in BALB/c and C57BL/6 Mice

BALB/c mice were used for estimation of the serological response to vaccination; C57BL/6 mice were used to determine the T cell-mediated immunity following vaccination.

Female BALB/c or C57BL/6 mice (Charles River, UK) received intradermal or intramuscular injections (via the left ear) with Compound 008 formulations on Day 0 and Day 21 and were sacrificed by CO₂ asphyxiation on Day 42. Midpoint blood samples were taken from each mouse on Day 7 and Day 28. On Day 42, blood from the mice was removed by cardiac puncture, transferred to sterile 1.5 mL centrifuge tubes, and allowed to clot for 30 min, and the serum was obtained via centrifugation at 10,000×g for 10 min. The serum was then transferred to clean 1.5 mL centrifuge tubes and stored at −20° C.

For T cell analysis—The spleen was aseptically resected from each C57BL/6 mouse and stored briefly on ice, in tissue preservation solution (Miltenyi Biotec, Germany Cat no. 130-100-008), before being mechanically disaggregation through 70 μm filter pores to obtain a single cell suspension. The cells were pelleted (280×g, 5 min, 4° C.), washed with ice-cold Phosphate buffered saline (PBS), and re-pelleted, before being resuspended in red blood cell lysis buffer (Life Technologies, UK Cat no. 00-4300-54). Red blood cell (RBC) lysis buffer was neutralised by the addition of an excess of ice-cold PBS, and cells were pelleted again. Cell pellets were resuspended in 10 mL of complete culture medium (Roswell Park Memorial Institute (RPMI), 10% heat-inactivated serum with 1% penicillin/streptomycin) in 10 cm cell culture dishes, and the culture dishes were incubated at 37° C., with 5% relative humidity. Cell count and viability was determined by flow cytometry using a Thermo Attune™ NxT Flow Cytometer, excluding cells that took up propidium iodide (Sigma Aldrich, UK Cat no. P4170). To investigate the T-cell response, splenocytes from C57BL/6 mice were subjected to IFNγ (Mabtech, Sweden Cat no. 3321-4APT-2/3321-4APW) and IL-2 ELISpot (Mabtech Sweden, 3441-2A0) assays. Briefly, 2.5×105 splenocytes were plated per well of ELISpot plates, and overlapping peptides (15 amino acids, 11 amino acid overlap; JPT, Germany) corresponding to the entire length of the respective antigens were used to stimulate the wells. The splenocyte cytokine production was analysed on a Mabtech IRIS ELISpot (Mabtech, Sweden) reader, with user-defined settings to exclude small spots with low intensity (size 300, intensity 250 units).

For IgG analysis—The presence of antigen-specific IgG (i.e. Spike, Membrane or Envelope) in sera from vaccinated BALB/c mice and Sprague Dawley rats was investigated by ELISA. Nunc Maxisorp 96 well plates (Thermofisher, UK Cat no. 439454) were coated with recombinant Spike, Membrane (both Bio-Techne, Germany Cat no. 10587-CV/Cat no. 10690-CV-100) or Envelope (Life Technologies, UK Cat no. RP-87682) by adding 50 μL of recombinant protein at 1 μg/mL (in ELISA coating buffer (Thermofisher, UK Cat no. CB01100), followed by a two-step incubation (1 h at 37° C. followed by overnight at 4° C.). Non-specific binding of serum components to wells was minimised by incubation with blocking solution (1% w/v bovine serum albumin (BSA) in PBS) for 1 h at room temperature. Samples were diluted as appropriate (e.g. 1:10, 1:100, 1:1000 in 1% w/v BSA in PBS), and standard curves were constructed (10000, 3333.3, 1111.1, 370.4, 123.5, 41.2 & 13.7 pg/mL) using commercial antibodies against Spike (Biotechne, Germany Cat no. mab12441-100), Membrane (Biotechne, Germany Cat no. mab10690-100) and Envelope (Abcam, UK Cat no. ab284658). Blocked wells were probed with diluted sera and, following three washes with PBS/0.05% Tween 20, wells were probed with a goat anti-mouse IgG (Abcam, UK Cat no. ab205719), diluted 1:10000 in 1% w/v BSA in PBS. Wells were washed three more times with PBS/0.05% Tween 20 before adding 100 μL/well of 3,3′,5,5′-tetramethylbenzidine (TMB) substrate. After 15 min colour development incubation, an equal quantity of stop solution was added, and absorbance read at 450 nm using a Molecular Devices SpectraMax iD3 plate reader. Further interrogations were made to determine the IgG2:IgG1 ratios using subtype-specific secondary antibodies (Thermofisher UK. Cat no: A10551/A-10685) at the same concentrations for mouse IgG analysis.

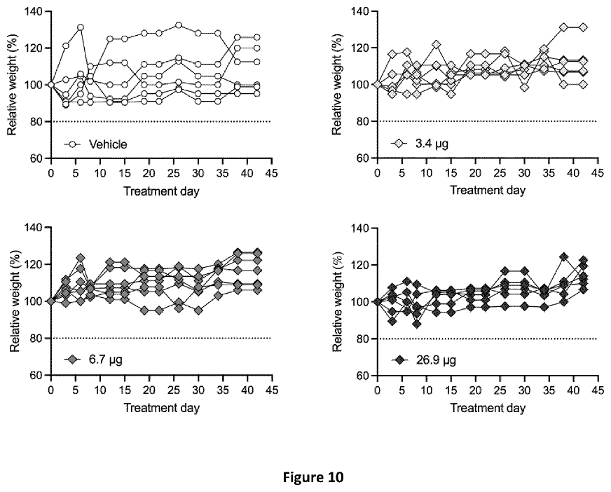

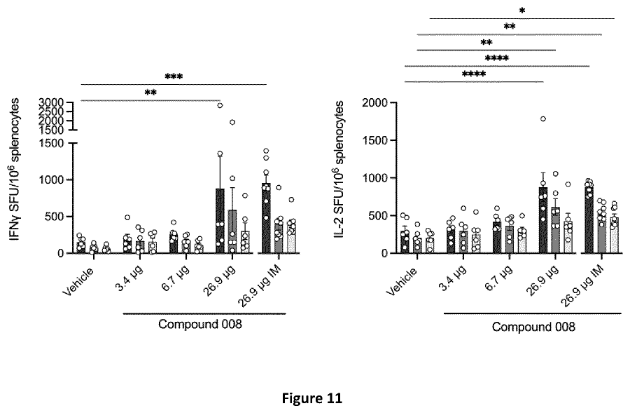

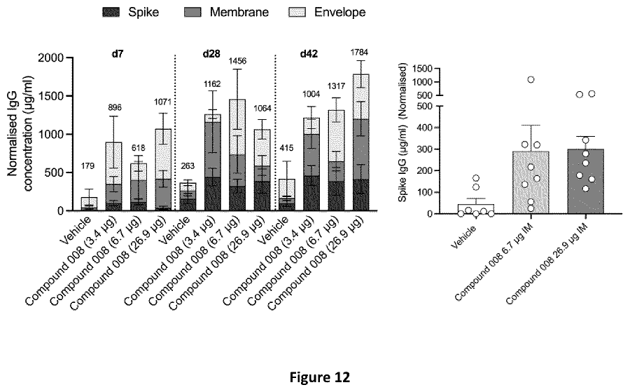

The results of Example 8 are shown in FIGS. 10, 11, 12, & 13.

### Example 9: Assessment of Immunogenicity of Compound 008 in Sprague Dawley Rats

Female Sprague Dawley rats (8-9 weeks old at outset) were assessed for their ability to generate a T cell-mediated response and an IgG-mediated response following treatment with Compound 008. As with immunogenicity studies in mice, treatments were administered on Days 0 and 21, with mid-study bleeds being taken at defined timepoints, and rats were culled on Day 42. Splenocytes were processed and stimulated in a similar manner to those from mice, and serum was analysed for antigen-specific IgG was also as described for mice, with the exception that a goat anti-rat secondary IgG (Thermo Fisher Catalogue #31470) was used.

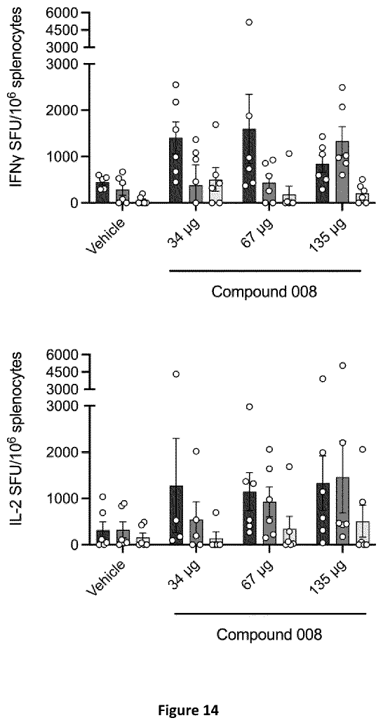

The results of Example 9 are shown in FIGS. 14 & 15.

### Example 10: Assessment of Circulating Toxicity Biomarkers in Rats Treated with Compound 008 and C57BL/6 Mice Treated with Compound 009

Circulating biomarkers that might indicate cardiac, renal and hepatic toxicity were assessed in the sera (Day 42) from vaccinated Sprague Dawley rats treated with Compound 008 or C57BL/6 mice treated with Compound 009 in the same regimen as described previously. The following assays were utilised: creatinine kinase (Abcam UK, Cat no. ab155901), lactate dehydrogenase (Abcam UK, Cat no. ab65393), uric acid (Abcam UK, Cat no. ab63544), creatinine (Abcam UK, Cat no. ab287790), urea (Abcam UK, Cat no. ab83362) alkaline phosphatase (Abcam UK, Cat no. ab285274), alanine transaminase (Abcam UK, Cat no. ab282882), albumin and total protein (Abcam UK, Cat no ab108792). All assays were performed following manufacturer's instructions, and the erythrocyte count was assessed microscopically.

Each BALB/c mouse was subjected to aseptic resection of the heart, lung, spleen, kidney and liver, which were then placed into individual tubes containing 5 mL of 10% (v/v) Neutral Buffered Formalin (Sigma UK, Cat. no HT501128), and supplied to the Royal Veterinary College (UK) for histological processing. The tissues underwent sequential incubation in alcohols to dehydrate them, followed by clearance using xylene and embedding in molten paraffin wax. The resulting blocks were sectioned and stained with hematoxylin and eosin. The resulting slides were then scanned using a Zeiss Axioscan and evaluated by a veterinary pathologist (Exe Pathology, UK) in a blinded manner. The pathological findings were scored using a non-linear semi-quantitative grading system.

The immunogenicity of Compound 009 (in terms of SpikeZA-specific IgG) generation was determined by ELISA in the same manner as described in Example 8.

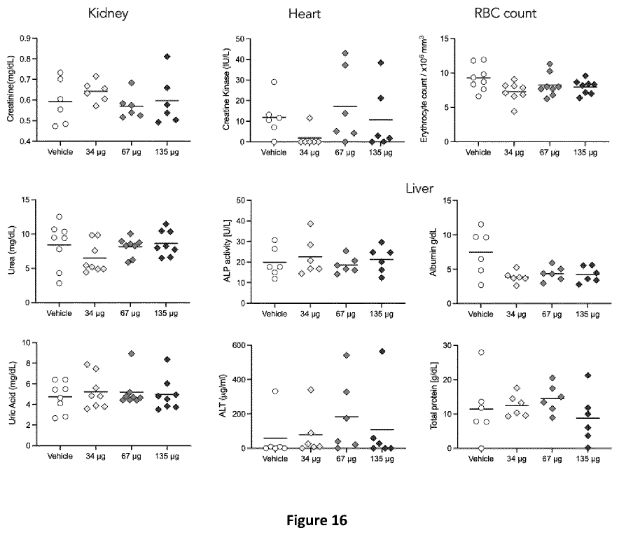

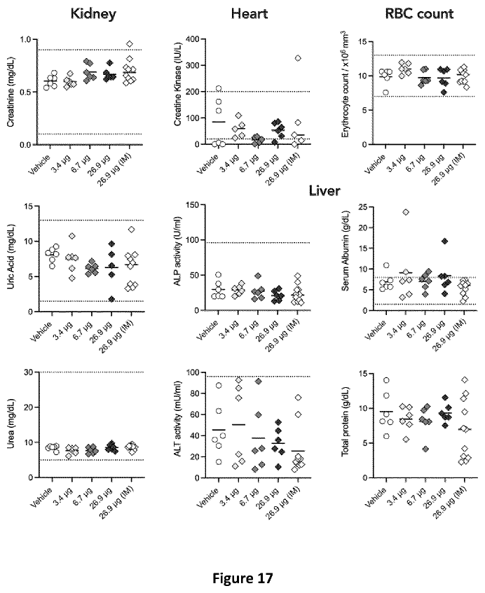

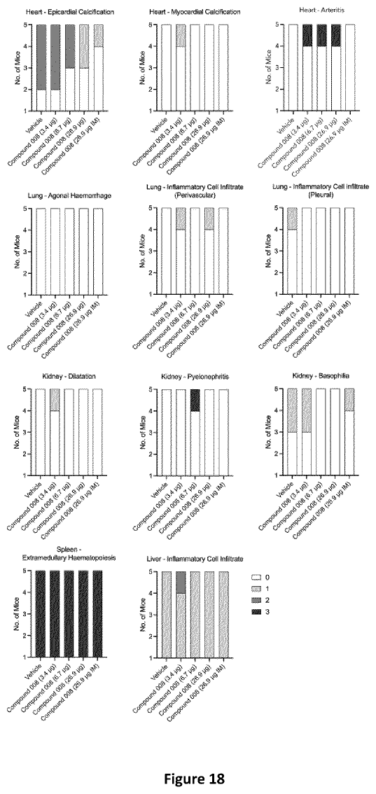

The results of Example 10 are shown in FIGS. 16, 17, 18 & 19.

### Example 11: mRNA Encapsulation of Compound 008

Nanoparticle encapsulation was measured via ionic exchange chromatography. 0.5 g SP-Sephadex (Sigma, Ger Cat no; SPC25120) was incubated overnight at room temperature in 10 mL 1M NaCl. The supernatant was removed, and resin was washed three times with 10 mL ultrapure (DNase/RNase free) water to eliminate any ionic solvent. Next, 2 mL of washed resin was loaded into a frit column and packed under pressure. Then, 20 μL of SME mRNA or Compound 008 at 1 mg/mL was loaded into the column and eluted with 3 mL of ultrapure (DNase/RNase free) water. Fractions were collected in 0.5 mL centrifuge tubes and analysed by UV-Vis using a NanoDrop-One spectrophotometer. Equivalent fractions with most nucleic acid content are compared in the analysis.

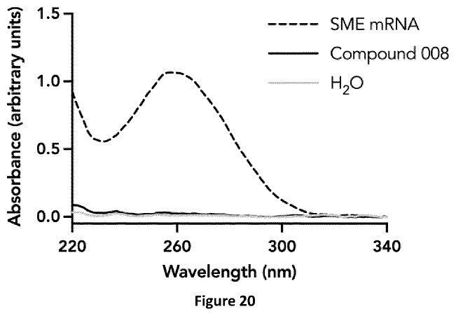

The results of Example 11 are shown in FIG. 20.

### Statements

Statement 1. A vaccine, comprising:
i) an mRNA polynucleotide comprising an open reading frame encoding a coronavirus antigen or an immunogenic fragment thereof; and
ii) an amphipathic cell penetrating peptide comprising or consisting of the amino acid sequence WEARLARALARALARHLARALARALRACEA (SEQ ID_No 1) or a sequence with at least 80% sequence identity or homology.
   Statement 2. A vaccine as stated in statement 1 further comprising a second mRNA polynucleotide comprising an open reading frame encoding a coronavirus antigen or an immunogenic fragment thereof.
   Statement 3. A vaccine as stated in statement 2 further comprising a third mRNA polynucleotide comprising an open reading frame encoding a coronavirus antigen or an immunogenic fragment thereof.
   Statement 4. A vaccine as stated in any one of the preceding statements wherein the open reading frame encodes a SARS-CoV-2 antigen or an immunogenic fragment thereof.
   Statement 5. A vaccine as stated in any one of the preceding statements wherein the open reading frame encodes a coronavirus M, E, S, or N protein or an immunogenic fragment thereof.
   Statement 6. A vaccine as stated in any one of statements 2-6 wherein the mRNA polynucleotides comprising open reading frames each encode a different coronavirus M, E, S, or N protein or an immunogenic fragment thereof.
   Statement 7. A vaccine comprising:
i) a first mRNA polynucleotide comprising an open reading frame comprising a sequence selected from SEQ ID_No 2 or a sequence with at least 95% sequence identity or homology;
ii) a second mRNA polynucleotide comprising an open reading frame comprising a sequence selected from SEQ ID_No 3 or a sequence with at least 95% sequence identity or homology;
iii) a third mRNA polynucleotide comprising an open reading frame comprising a sequence selected from SEQ ID_No 4 or a sequence with at least 95% sequence identity or homology;
iv) an amphipathic cell penetrating peptide comprising the amino acid sequence WEARLARALARALARHLARALARALRACEA (SEQ ID_No 1) or a sequence with at least 95% sequence identity or homology.
   Statement 8. A vaccine comprising:
i) a first mRNA polynucleotide comprising an open reading frame encoding an antigen selected from SEQ ID_No 6 or a sequence with at least 95% sequence identity or homology;
ii) a second mRNA polynucleotide comprising an open reading frame encoding an antigen selected from SEQ ID_No 7 or a sequence with at least 95% sequence identity or homology;
iii) a third mRNA polynucleotide comprising an open reading frame encoding an antigen selected from SEQ ID_No 8 or a sequence with at least 95% sequence identity or homology;
iv) an amphipathic cell penetrating peptide comprising the amino acid sequence WEARLARALARALARHLARALARALRACEA (SEQ ID_No 1) or a sequence with at least 95% sequence identity or homology.
   Statement 9. A vaccine as stated in any one of statements 2-8 wherein the mRNA polynucleotides are present in an equal copy number ratio.
   Statement 10. A vaccine as stated in any one of statements 2-8 wherein the mRNA polynucleotides are present in an unequal copy number ratio.
   Statement 11. A vaccine as stated in any one of the preceding statements wherein the amphipathic cell penetrating peptide comprises the amino acid sequence WEARLARALARALARHLARALARALRACEA (SEQ ID_No 1).
   Statement 12. A vaccine as stated in any one of the preceding statements wherein the amphipathic cell penetrating peptide consists of the amino acid sequence WEARLARALARALARHLARALARALRACEA (SEQ ID_No 1).
   Statement 13. A vaccine as stated in any one of the preceding statements wherein the vaccine further comprises an mRNA polynucleotide comprising an open reading frame encoding an immunoadjuvant.
   Statement 14. A vaccine as stated in statement 13 wherein the immunoadjuvant is a cytokine or granulocyte-macrophage colony-stimulating factor.
   Statement 15. A vaccine as stated in any one of the preceding statements wherein the mRNA polynucleotide comprises a five-prime cap.
   Statement 16. A vaccine as stated in any one of the preceding statements wherein the mRNA polynucleotide comprises modified uridine nucleotides wherein all uridine nucleotides are N1-methylpseudouridine nucleotides.
   Statement 17. A vaccine as stated in any one of the preceding statements wherein the mRNA polynucleotide comprises unmodified cytidine nucleotides.
   Statement 18. A vaccine as stated in any one of the preceding statements wherein the vaccine comprises nanoparticles.
   Statement 19. A vaccine as stated in any one of the preceding statements wherein the N:P ratio of amphipathic cell penetrating peptide:mRNA polynucleotide in the vaccine is about 8.5-9.5:1.
   Statement 20. A vaccine as stated in any one of the preceding statements further comprising trehalose.
   Statement 21. A method of preparing a nanoparticle formulation as stated in any one of statements 1-20 which comprises formulating a solution of an mRNA polynucleotide with an amphipathic cell penetrating peptide in an automated controlled mixing system.
   Statement 22. A vaccine as stated in statements 1-20 for use in the treatment of coronavirus infection.
   Statement 23. A vaccine as stated in statements 1-20 for use in the treatment of SARS-CoV-2 infection.
   Statement 24. A method of treating COVID-19 in a warm-blooded animal, such as man, which comprises administering to said animal an effective amount of a vaccine as stated in in statements 1-20.

1. A vaccine, comprising:
i) an mRNA polynucleotide comprising an open reading frame encoding a coronavirus antigen or an immunogenic fragment thereof; and
ii) an amphipathic cell penetrating peptide comprising or consisting of the amino acid sequence WEARLARALARALARHLARALARALRACEA (SEQ ID_No 1) or a sequence with at least 80% sequence identity or homology.

2. The vaccine as claimed in claim 1 further comprising a second mRNA polynucleotide comprising an open reading frame encoding a coronavirus antigen or an immunogenic fragment thereof.

3. The vaccine as claimed in claim 2 further comprising a third mRNA polynucleotide comprising an open reading frame encoding a coronavirus antigen or an immunogenic fragment thereof.

4. The vaccine as claimed in claim 1 wherein the open reading frame encodes a SARS-CoV-2 antigen or an immunogenic fragment thereof.

5. The vaccine as claimed in claim 1 wherein the open reading frame encodes a coronavirus M, E, S, or N protein or an immunogenic fragment thereof.

6. The vaccine as claimed in claim 2 wherein the mRNA polynucleotides comprising open reading frames each encode a different coronavirus M, E, S, or N protein or an immunogenic fragment thereof.

7. A vaccine comprising:
i) a first mRNA polynucleotide comprising an open reading frame comprising a sequence selected from SEQ ID_No 2 or SEQ ID_No 5 or a sequence with at least 95% sequence identity or homology;
ii) a second mRNA polynucleotide comprising an open reading frame comprising a sequence selected from SEQ ID_No 3 or a sequence with at least 95% sequence identity or homology;
iii) a third mRNA polynucleotide comprising an open reading frame comprising a sequence selected from SEQ ID_No 4 or a sequence with at least 95% sequence identity or homology;
iv) an amphipathic cell penetrating peptide comprising the amino acid sequence WEARLARALARALARHLARALARALRACEA (SEQ ID_No 1) or a sequence with at least 95% sequence identity or homology.

8. A vaccine comprising:
i) a first mRNA polynucleotide comprising an open reading frame encoding an antigen selected from SEQ ID_No 6 or SEQ ID_No 9 or a sequence with at least 95% sequence identity or homology;
ii) a second mRNA polynucleotide comprising an open reading frame encoding an antigen selected from SEQ ID_No 7 or a sequence with at least 95% sequence identity or homology;
iii) a third mRNA polynucleotide comprising an open reading frame encoding an antigen selected from SEQ ID_No 8 or a sequence with at least 95% sequence identity or homology;
iv) an amphipathic cell penetrating peptide comprising the amino acid sequence WEARLARALARALARHLARALARALRACEA (SEQ ID_No 1) or a sequence with at least 95% sequence identity or homology.

9. The vaccine as claimed in claim 2 wherein the mRNA polynucleotides are present in an equal copy number ratio.

10. The vaccine as claimed in claim 2 wherein the mRNA polynucleotides are present in an unequal copy number ratio.

11. The vaccine as claimed in claim 1 wherein the amphipathic cell penetrating peptide comprises the amino acid sequence WEARLARALARALARHLARALARALRACEA (SEQ ID_No 1).

12. The vaccine as claimed in claim 1 wherein the amphipathic cell penetrating peptide consists of the amino acid sequence WEARLARALARALARHLARALARALRACEA (SEQ ID_No 1).

13. The vaccine as claimed in claim 1 wherein the mRNA polynucleotide comprises a five-prime cap.

14. The vaccine as claimed in claim 1 wherein the mRNA polynucleotide comprises modified uridine nucleotides wherein all uridine nucleotides are N1-methylpseudouridine nucleotides.

15. The vaccine as claimed in claim 1 wherein the mRNA polynucleotide comprises unmodified cytidine nucleotides.

16. The vaccine as claimed in claim 1 wherein the vaccine comprises nanoparticles.

17. The vaccine as claimed in claim 1 wherein the N:P ratio of amphipathic cell penetrating peptide:mRNA polynucleotide in the vaccine is about 8.5-9.5:1.

18. The vaccine as claimed in claim 1 further comprising trehalose.

19. The vaccine as claimed in claim 1 comprising:
(i) 0.01-1.5% w/w one or more mRNA polynucleotides comprising open reading frames encoding a coronavirus antigen or an immunogenic fragment thereof,
(ii) 1-15% w/w an amphipathic cell penetrating peptide comprising or consisting of the amino acid sequence WEARLARALARALARHLARALARALRACEA (SEQ ID_No 1) or a sequence with at least 80% sequence identity or homology; and
(iii) 80-99% w/w trehalose.

20. The vaccine in claim 1 comprising:
(i) 0.01-1.5% w/w one or more mRNA polynucleotides comprising open reading frames encoding a coronavirus S, M and/or E protein or an immunogenic fragment thereof,
(ii) 1-15% w/w an amphipathic cell penetrating peptide comprising or consisting of the amino acid sequence WEARLARALARALARHLARALARALRACEA (SEQ ID_No 1) or a sequence with at least 80% sequence identity or homology; and
(iii) 80-99% w/w trehalose;
formulated as an isotonic formulation in water with an osmolality of 290-310 mOsmol/kg.

21. The method of preparing a nanoparticle formulation as claimed in claim 1 which comprises formulating a solution of an mRNA polynucleotide with an amphipathic cell penetrating peptide in an automated controlled mixing system.

22. The vaccine as claimed in claim 1 for use in the treatment of coronavirus infection.

23. The vaccine as claimed in claim 1 for use in the treatment of SARS-CoV-2 infection.

24. The method of treating COVID-19 in a warm-blooded animal, such as man, which comprises administering to said animal an effective amount of a vaccine as claimed in claim 1.


SEQUENCE LISTING (13 sequences)

SEQ ID NO: 1 - protein, 30 aa, synthetic construct
WEARLARALARALARHLARALARALRACEA

SEQ ID NO: 2 - RNA, 3822 nt, synthetic construct
  source 1..3822: mol_type = other RNA
  modified_base 1..3822: mod_base = m1f
atgtttgtgttcctggttttactgccactagtctccagtcagtgtgtgaatctgacaaccagaacccagctcccccctgcctacactaattctttcacacgtggggtttattaccctgacaaagttttccggtcctcagtgttgcacagcacccaggacttgttcctccctttcttttccaatgtgacttggttccatgctatacatgtctctgggaccaatgggaccaagaggtttgacaaccctgtcctaccattcaatgacggtgtgtactttgcttccaccgagaagtctaacataatccgaggctggatttttggtaccactttagactcgaagacccagtccctacttattgtgaataacgccactaatgtggtaattaaagtctgtgagtttcagttttgtaatgatccattcttgggtgtgtactaccacaagaacaacaagagttggatggagagtgagttcagagtgtattccagcgcgaataactgcaccttcgagtacgtctctcagcccttccttatggacctcgaaggaaaacagggtaacttcaagaatctgagggagttcgtgtttaagaatatcgatgggtatttcaagatctactctaagcacacgccgattaatctggtgagagatctcccgcagggtttctcggccttggagccactggtggatctgccaatagggattaacatcactaggttccaaacgttacttgctttacacagaagttatctaacccctggggatagctcatccgggtggacagctggtgcggcagcttattacgtgggttacctgcagcctcgaaccttcctactgaaatacaacgaaaatggaaccattaccgatgccgtagactgtgcacttgacccactctcagaaacaaagtgcacgctgaagtccttcaccgtagagaaaggaatctaccagactagtaacttccgagtccagccgacagagtctatcgttagatttcccaacatcacaaacttgtgcccgtttggcgaagtgttcaacgccacccggtttgcatctgtctatgcttggaacaggaagcgcatcagcaactgtgtggccgattacagcgtcctatacaactccgccagcttttccacttttaagtgttatggagtgagccctactaaactgaatgacctctgctttactaacgtctatgcagattcgttcgtaatcagaggcgatgaggtcagacagatcgccccagggcaaactggaaagatcgcggactataactataaactgccagacgattttacaggctgcgtgatagcttggaactctaacaatcttgattctaaggtggggggcaactataattacctgtatcgattgtttaggaagagcaatctcaagccttttgagagagacatttcgacggagatctaccaggccggaagcacaccttgcaatggcgttgaggggttcaattgttacttccctctgcagtcatatggcttccagcccactaatggcgttggataccaaccctaccgagtagtagtgcttagttttgaactcctgcacgcaccagccaccgtttgcggacccaaaaagtccacgaatctggtgaagaacaagtgtgtcaatttcaacttcaatgggctcacaggcacaggggttctgactgagtccaacaagaagtttctgcctttccagcagttcggccgcgacattgctgacactactgatgctgtccgcgacccacagaccttggagattctggacatcaccccctgctctttcgggggcgtcagcgtgataaccccaggaacaaatacctccaaccaggtggccgtgctgtatcagggggtgaactgcacagaagtccctgtggctattcatgccgaccaacttactcctacctggcgggtgtatagcacaggctcgaatgttttccagacccgggcaggctgtttgatcggggctgaacatgtcaacaactcatatgagtgtgacatacccatcggtgcaggaatctgcgctagctaccagactcagactaactctccccggcgggcacgtagtgtagccagtcagtccatcattgcctacactatgtcactgggtgccgaaaattcagtggcctactctaacaacagcattgccatacccacaaattttacgattagtgttaccacagaaattctaccagtgtctatgaccaagacctcagtagattgtacaatgtacatctgtggtgattcaacggaatgcagcaatctgctgctgcagtatggcagtttctgcacacagctgaaccgcgctttaaccggaatagccgttgagcaggacaaaaacacccaggaagttttcgcacaggtcaagcagatctacaaaacccccccaatcaaagactttggaggtttcaactttagccaaatactcccggatccatcaaagcccagcaagaggtcctttattgaagatctactcttcaacaaagtgacactggccgatgctggcttcatcaaacagtatggcgattgccttggtgatatcgcagcgcgggacctcatttgcgcacagaagtttaacggccttactgtgctgccacccttgctcaccgacgaaatgattgctcagtacacttccgcactgttagcggggacaatcacgagcgggtggacctttggcgcaggcgccgccttgcagataccattcgctatgcaaatggcttacaggtttaatgggatcggagtgacacagaatgtgctctatgagaaccaaaaactgatcgccaaccaatttaacagtgctatcggcaaaattcaagactcactttcttccacagcgagtgcccttggaaaactgcaagatgtggtcaaccaaaatgcacaggctctgaacacgctggttaaacaactcagctccaactttggagcaattagcagtgtgctgaatgatatccttagccgtctggacccccccgaggcggaagtgcagattgataggctgatcacaggcagacttcagagtctgcagacatatgtgactcaacagctgattcgggccgcagaaatcagagcctctgccaatctggctgctactaagatgtcagagtgcgtccttggacagtcaaaacgcgtggatttttgtggaaagggctaccacctcatgtccttccctcagtcagcacctcacggcgtcgtcttcttgcatgtgacatacgtccccgcacaagagaagaacttcacaactgctcctgccatctgtcacgatggaaaggcacacttcccccgcgagggtgtctttgtgtccaatggcacccactggtttgtaacacaaaggaatttctatgagccccaaatcatcactacggacaacacattcgtgtctgggaactgtgacgtggtaatcggaattgtcaacaacacagtttatgaccctctgcagcctgaattggactccttcaaggaggagttagataaatactttaagaaccataccagcccagacgttgacctcggtgacatctctggcattaacgccagcgttgtaaacattcagaaagaaattgaccgcctcaatgaggtggccaagaatctgaatgaatctctcatcgatctccaggaactgggaaagtatgagcagtacataaagtggccgtggtacatctggctaggttttatcgctggcttgattgccatagtaatggtgacaatcatgttgtgctgtatgaccagttgctgtagctgcctcaagggctgttgcagctgtggatcctgctgcaaatttgatgaagacgacagcgagccagtgctcaaaggagtcaaactgcattacacctga

SEQ ID NO: 3 - RNA, 669 nt, synthetic construct
  source 1..669: mol_type = other RNA
  modified_base 1..669: mod_base = m1f
atggcagattccaacggcaccattaccgtcgaagagcttaaaaagctcctggaacaatggaacctagttataggcttcctattcctgacatggatttgtctgctgcagtttgcctatgccaacaggaataggtttttgtatataattaagctgattttcctctggctgctgtggccagtaactttagcttgttttgtgcttgctgctgtgtacagaataaattggatcaccgggggaatcgcaatcgcaatggcttgccttgtgggcttgatgtggctcagctacttcatcgcttctttcagactgtttgcgcggacccgatccatgtggtctttcaatcccgagacaaacatccttctcaacgtgcccctccacggcactattctgaccagaccgttactcgaatcggagctcgtcatcggagccgtgatcctgcgtggacatctgcggatcgccgggcaccacctaggacgctgcgatatcaaggacctgcctaaagagatcactgtggccacatcacgaacgctttcttattacaaattgggggcgagccagcgggttgcaggtgactcaggttttgccgcatacagtcgctacaggattggcaactataagctgaacacagaccattccagcagcagtgacaatattgccttgctggtccagtga

SEQ ID NO: 4 - RNA, 228 nt, synthetic construct
  source 1..228: mol_type = other RNA
  modified_base 1..228: mod_base = m1f
atgtactcattcgtatccgaagagaccggcacgttaatcgtcaacagcgtactgctttttttggctttcgtggtcttcctgctcgtgacactagccatcctcactgcccttaggctgtgtgcatactgctgcaatattgttaacgtgagtctggtgaaaccatccttttatgtgtacagccgggtgaagaatctgaacagctctagagttcccgacctcctggtctga

SEQ ID NO: 5 - RNA, 3813 nt, synthetic construct
  source 1..3813: mol_type = other RNA
  modified_base 1..3813: mod_base = m1f
atgttcgtgttcctcgtgctgctgcccctggtgtctagccagtgcgtgaacctgaccaccaggacacagctgcctccagcctacaccaacagcttcaccaggggagtgtactaccccgacaaggtgttccggagcagcgtgctgcatagcacccaggacctgttcctgcccttcttcagcaacgtgacttggttccacgccatccacgtcagcggaacaaacggcaccaagcgcttcgccaacccagtgctgcccttcaacgacggagtgtacttcgccagcaccgagaagagcaacatcatccggggttggatcttcggcaccacactggacagcaagacccagagcctgctgatcgtgaacaacgccaccaacgtcgtgatcaaggtctgcgagttccagttttgcaacgaccccttcctgggcgtgtactaccacaagaacaacaagtcttggatggagagcgagttccgggtgtacagcagcgccaacaattgcaccttcgagtacgtgtcccagcccttcctgatggacctggaaggcaagcagggcaacttcaagaacctgcgggagttcgtgttcaagaacatcgacggctacttcaagatctacagcaagcacacccccatcaacctcgtgagaggcctgccccagggattctcagctctggaacctctggtggacctgcctatcggcatcaacatcacccggttccagaccctgcataggtcctacctgaccccaggagactcttctagcggctggacagcaggagcagccgcttactacgtgggatacctgcagcctcggaccttcctgctgaagtacaacgagaacggcaccatcacagacgccgtggattgcgctctggatcctctgagcgagaccaagtgcaccctgaagagcttcaccgtggagaagggcatctaccagaccagcaacttccgggtgcagcctacagagagcatcgtgcggttccctaacatcaccaacctctgccccttcggcgaggtgttcaacgccaccagattcgccagcgtgtacgcttggaaccggaagcggatcagcaactgcgtggccgactacagcgtgctgtacaacagcgccagcttcagcaccttcaagtgctacggcgtgtcccctaccaagctgaacgatctgtgcttcaccaacgtgtacgccgacagcttcgtgatcaggggagacgaagtgcgccagatcgctccaggacagacaggaaacatcgccgactacaactacaagctgcccgacgacttcaccggttgcgtgatcgcttggaacagcaacaacctggacagcaaggtcggcggcaactacaactacctgtaccggctgttccggaagagcaacctgaagcccttcgagcgggacatcagcacagagatctaccaggccggcagcacaccttgtaacggcgtgaagggcttcaactgctacttccctctgcagagctacggcttccagcctacatacggcgtgggataccagccttacagggtggtggtgctgagcttcgaactgctgcacgctccagctaccgtctgcggacctaagaagagcaccaacctggtgaagaacaagtgcgtgaatttcaacttcaacggcctgaccggaaccggcgtgctgaccgagagcaacaagaagttcctgcccttccagcagttcggcagagacatcgcagacaccacagacgccgtgagagatcctcagaccctggagatcctggacatcaccccttgcagcttcggaggagtgtccgtgatcaccccaggaaccaacaccagcaatcaggtggccgtgctgtaccagggagtgaattgcaccgaggtgccagtggctattcacgccgatcagctgacccctacttggagagtgtacagcaccggcagcaacgtgttccagaccagagccggttgcctgatcggagccgagcacgtgaacaacagctacgagtgcgacatccctatcggagccggaatctgcgctagctaccagacccagaccaacagccctagaagagccagaagcgtggcctctcagagcatcatcgcctacaccatgagcctgggagtcgaaaacagcgtggcctacagcaacaacagcatcgccatccccaccaacttcaccatcagcgtgacaaccgagatcctgccagtgtccatgaccaagaccagcgtcgactgcaccatgtacatctgcggcgacagcaccgagtgctctaacctgctgctgcagtacggctccttttgcacccagctgaacagggctctgacaggcatcgcagtggagcaggacaagaacacccaggaggtcttcgcccaggtcaagcagatctacaagaccccccctatcaaggacttcggaggcttcaacttcagccagatcctgcccgaccctagcaagcctagcaagcggagcttcatcgaggacctgctgttcaacaaggtcaccctggccgacgcaggcttcatcaagcagtacggcgattgcctgggcgacatcgccgctagagacctgatttgcgcccagaagttcaacggcctgacagtgctgcctcctctgctgaccgacgagatgatcgctcagtacacaagcgctctgctggcaggaaccatcaccagcggttggacattcggagccggagccgctctgcagatcccttttgccatgcagatggcctaccgcttcaacggaatcggcgtgacccagaacgtgctctacgagaaccagaagctgatcgccaaccagttcaacagcgccatcggcaagatccaggacagcctgagcagcacagctagcgctctgggaaagctgcaggacgtggtgaaccagaacgcccaggctctgaacaccctggtgaagcagctgagcagcaacttcggagccatcagcagcgtgctgaacgacatcctgagcagactggaccctccagaagccgaagtgcagatcgacaggctgatcaccggcagactgcagtctctgcagacctacgtgacccagcagctgatcagggccgccgaaatcagagctagcgctaacctggccgccacaaagatgagcgagtgcgtgctgggacagagcaagagagtggacttctgcggcaagggctaccacctgatgagctttcctcagagcgctcctcacggagtggtgtttctgcacgtgacctacgtgccagcccaggagaagaacttcaccaccgctccagccatttgccacgacggaaaggcccacttccccagagaaggcgtgttcgtgtccaacggcacccattggttcgtgacccagcggaacttctacgagccccagatcatcaccaccgacaacaccttcgtgagcggcaattgcgacgtggtcatcggcatcgtgaacaacaccgtgtacgaccccctgcagccagagctggactccttcaaggaggagctggacaagtacttcaagaaccacaccagcccagacgtggacctgggagatatcagcggcatcaacgccagcgtggtgaacatccagaaggagatcgacaggctgaacgaggtggccaagaacctgaacgagagcctgatcgacctgcaggagctgggcaagtacgagcagtacatcaagtggccttggtacatttggctgggcttcatcgccggcctgatcgcaatcgtcatggtgaccatcatgctctgttgcatgaccagttgctgcagctgcctgaagggttgctgcagttgcgggagttgctgcaagttcgacgaggacgacagcgagccagtgctgaagggagtgaagctgcactacacctag

SEQ ID NO: 6 - protein, 1273 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 7 - protein, 222 aa, synthetic construct
MADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQ

SEQ ID NO: 8 - protein, 75 aa, synthetic construct
MYSFVSEETGTLIVNSVLLFLAFVVFLLVTLAILTALRLCAYCCNIVNVSLVKPSFYVYSRVKNLNSSRVPDLLV

SEQ ID NO: 9 - protein, 1270 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFANPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRGLPQGFSALEPLVDLPIGINITRFQTLHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGVENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 10 - RNA, 489 nt, synthetic construct
  source 1..489: mol_type = other RNA
  modified_base 1..489: mod_base = m1f
atgagaatcagcaagccgcacctgagaagcatcagcattcagtgctacctctgcctgctgctcaacagccacttcctgaccgaagccggaatccacgtattcatcctgggatgcttcagcgccggcctccccaaaacagaggcaaactgggtaaacgtaataagcgacctcaaaaagatcgaagacctgatccagagcatgcacatcgacgcaaccctgtacaccgaaagcgacgtacacccaagctgcaaagtaaccgcgatgaaatgcttcctgctggagctgcaagtaatcagcctggagagcggcgacgccagcatccacgacaccgtggaaaacctgattatcttggcaaacaacagcctgagcagcaacggaaacgtgacagagagcggatgcaaagagtgcgaggagctggaggaaaagaacatcaaagaattcctccaaagcttcgtgcacatagtgcagatgttcataaacacaagctga

SEQ ID NO: 11 - RNA, 435 nt, synthetic construct
  source 1..435: mol_type = other RNA
  modified_base 1..435: mod_base = m1f
atgtggctgcaaagccttctgctcttggggactgtggcctgctcaatttctgcacctgcccgatcgcccagtccaagcacgcagccctgggagcatgtgaatgccatccaggaggcgcggaggttactgaacctgagtagagacactgctgctgagatgaatgaaacagtagaagtgatatcagaaatgtttgatctccaggagcctacctgcctacagacacgcctggagctgtataaacagggcctgaggggctccctcaccaagctcaaaggacccttgacaatgatggccagccactacaagcaacactgtcctccaaccccggaaacttcctgtgcaacccagattatcacatttgaatccttcaaagagaacctgaaggatttcctacttgttatccccttcgactgctgggagccagtccaggagtga

SEQ ID NO: 12 - protein, 162 aa, synthetic construct
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 13 - protein, 144 aa, synthetic construct
MWLQSLLLLGTVACSISAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE